SEQUENCE LISTING (28 sequences)

SEQ ID NO: 1 - DNA, 401 bp, Artificial Sequence
  human synapsin 1 promoter, truncated version
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctc

SEQ ID NO: 2 - DNA, 422 bp, Artificial Sequence
  human synapsin 1 promoter with 5′ extension
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctc

SEQ ID NO: 3 - DNA, 448 bp, Artificial Sequence
  human synapsin 1 promoter with 3′ extension
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 4 - DNA, 851 bp, Artificial Sequence
  eSYN promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtcgcgactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagg

SEQ ID NO: 5 - DNA, 253 bp, Artificial Sequence
  human beta-actin promoter
gcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagtc

SEQ ID NO: 6 - DNA, 281 bp, Artificial Sequence
  chicken beta-actin promoter
ggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggga

SEQ ID NO: 7 - DNA, 573 bp, Artificial Sequence
  CAG promoter
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 8 - DNA, 220 bp, Artificial Sequence
  CMV promoter
tggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg

SEQ ID NO: 9 - DNA, 583 bp, Artificial Sequence
  CMV promoter (second version)
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgt

SEQ ID NO: 10 - DNA, 281 bp, Artificial Sequence
  human CAMKII promoter
acttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgc

SEQ ID NO: 11 - DNA, 455 bp, Artificial Sequence
  human EF1-alpha promoter
caacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaatagcatgcgtgaggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgtt

SEQ ID NO: 12 - DNA, 2120 bp, Artificial Sequence
  human SLC6A3 gene
atgagtaagagcaaatgctccgtgggactcatgtcttccgtggtggccccggctaaggagcccaatgccgtgggcccgaaggaggtggagctcatccttgtcaaggagcagaacggagtgcagctcaccagctccaccctcaccaacccgcggcagagccccgtggaggcccaggatcgggagacctggggcaagaagatcgactttctcctgtccgtcattggctttgctgtggacctggccaacgtctggcggttcccctacctgtgctacaaaaatggtggcggtgccttcctggtcccctacctgctcttcatggtcattgctgggatgccacttttctacatggagctggccctcggccagttcaacagggaaggggccgctggtgtctggaagatctgccccatactgaaaggtgtgggcttcacggtcatcctcatctcactgtatgtcggcttcttctacaacgtcatcatcgcctgggcgctgcactatctcttctcctccttcaccacggagctcccctggatccactgcaacaactcctggaacagccccaactgctcggatgcccatcctggtgactccagtggagacagctcgggcctcaacgacacttttgggaccacacctgctgccgagtactttgaacgtggcgtgctgcacctccaccagagccatggcatcgacgacctggggcctccgcggtggcagctcacagcctgcctggtgctggtcatcgtgctgctctacttcagcctctggaagggcgtgaagacctcagggaaggtggtatggatcacagccaccatgccatacgtggtcctcactgccctgctcctgcgtggggtcaccctccctggagccatagacggcatcagagcatacctgagcgttgacttctaccggctctgcgaggcgtctgtttggattgacgcggccacccaggtgtgcttctccctgggcgtggggttcggggtgctgatcgccttctccagctacaacaagttcaccaacaactgctacagggacgcgattgtcaccacctccatcaactccctgacgagcttctcctccggcttcgtcgtcttctccttcctggggtacatggcacagaagcacagtgtgcccatcggggacgtggccaaggacgggccagggctgatcttcatcatctacccggaagccatcgccacgctccctctgtcctcagcctgggccgtggtcttcttcatcatgctgctcaccctgggtatcgacagcgccatgggtggtatggagtcagtgatcaccgggctcatcgatgagttccagctgctgcacagacaccgtgagctcttcacgctcttcatcgtcctggcgaccttcctcctgtccctgttctgcgtcaccaacggtggcatctacgtcttcacgctcctggaccattttgcagccggcacgtccatcctctttggagtgctcatcgaagccatcggagtggcctggttctatggtgttgggcagttcagcgacgacatccagcagatgaccgggcagcggcccagcctgtactggcggctgtgctggaagctggtcagcccctgctttctcctgttcgtggtcgtggtcagcattgtgaccttcagacccccccactacggagcctacatcttccccgactgggccaacgcgctgggctgggtcatcgccacatcctccatggccatggtgcccatctatgcggcctacaagttctgcagcctgcctgggtcctttcgagagaaactggcctacgccattgcacccgagaaggaccgtgagctggtggacagaggggaggtgcgccagttcacgctccgccactggctcaaggtgtagagggagcagagacgaagaccccaggaagtcatcctgcaatgggagagacacgaacaaaccaaggaaatctaagtttcgagagaaaggagggcaacttctactcttcaacctctactgaaaacacaaacaacaaagcagaagactcctctcttctgactgtttacacctttccgtgccgggagcgcacctcgccgtgtcttgtgttgctgtaataacgacgtagatctgtgcagcgaggtccaccccgttgttgtccc

SEQ ID NO: 13 - protein, 620 aa, Artificial Sequence
  human DAT protein
MSKSKCSVGLMSSVVAPAKEPNAVGPKEVELILVKEQNGVQLTSSTLTNPRQSPVEAQDRETWGKKIDFLLSVIGFAVDLANVWRFPYLCYKNGGGAFLVPYLLFMVIAGMPLFYMELALGQFNREGAAGVWKICPILKGVGFTVILISLYVGFFYNVIIAWALHYLFSSFTTELPWIHCNNSWNSPNCSDAHPGDSSGDSSGLNDTFGTTPAAEYFERGVLHLHQSHGIDDLGPPRWQLTACLVLVIVLLYFSLWKGVKTSGKVVWITATMPYVVLTALLLRGVTLPGAIDGIRAYLSVDFYRLCEASVWIDAATQVCFSLGVGFGVLIAFSSYNKFTNNCYRDAIVTTSINSLTSFSSGFVVFSFLGYMAQKHSVPIGDVAKDGPGLIFIIYPEAIATLPLSSAWAVVFFIMLLTLGIDSAMGGMESVITGLIDEFQLLHRHRELFTLFIVLATFLLSLFCVTNGGIYVFTLLDHFAAGTSILFGVLIEAIGVAWFYGVGQFSDDIQQMTGQRPSLYWRLCWKLVSPCFLLFVVVVSIVTFRPPHYGAYIFPDWANALGWVIATSSMAMVPIYAAYKFCSLPGSFREKLAYAIAPEKDRELVDRGEVRQFTLRHWLKV

SEQ ID NO: 14 - DNA, 293 bp, Artificial Sequence
  CMV immediate early enhancer
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacca

SEQ ID NO: 15 - DNA, 512 bp, Artificial Sequence
  TPL-eMLP adenovirus derived enhancer element
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccag

SEQ ID NO: 16 - DNA, 476 bp, Artificial Sequence
  Human beta-globin intron
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 17 - DNA, 953 bp, Artificial Sequence
  Human beta-actin exon/intron
cgcgtccgcccgcgagcacagagcctcgcctttgccgatccgccgcccgtccacacccgccgccaggtaagcccggccagccgaccggggcatgcggccgcggcccttcgcccgtgcagagccgccgtctgggccgcagcggggggcgcatggggcggaaccggaccgccgtggggggcgcgggagaagcccctgggcctccggagatgggggacaccccacgccagttcgcaggcgcgaggccgcgctcgggcgggcgcgctccgggggtgccgctctcggggcgggggcaaccggcggggtctttgtctgagccgggctcttgccaatggggatcgcacggtgggcgcggcgtagcccccgtcaggcccggtgggggctggggcgccatgcgcgtgcgcgctggtcctttgggcgctaactgcgtgcgcgctgggaattggcgctaattgcgcgtgcgcgctgggactcaatggcgctaatcgcgcgtgcgttctggggcccgggcgcttgcgccacttcctgcccgagccgctggcgcccgagggtgtggccgctgcgtgcgcgcgcgcgacccggtcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcggcagcggctgctcttggcggccccgaggtgactatagccttcttttgtgtcttgatagttcgccagcctctgctaaccatgttcatgccttcttctttttcctacagctcctgggcaacgtgctggttattgtgctgtctcatcattttggcaaagaattc

SEQ ID NO: 18 - DNA, 956 bp, Artificial Sequence
  Human EF1-alpha intron/exon
ctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacgtgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcag

SEQ ID NO: 19 - DNA, 939 bp, Artificial Sequence
  Human EF1-alpha, intron A
gtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 20 - DNA, 1068 bp, Artificial Sequence
  Chicken beta-actin exon/intron + rabbit globin intron
gtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 21 - DNA, 121 bp, Artificial Sequence
  CMV IE exon
tcagatcgcctggagaggccatccacgctgttttgacctccatagtggacaccgggaccgatccagcctccgcggccgggaacggtgcattggaacgcggattccccgtgccaagagtgac

SEQ ID NO: 22 - DNA, 126 bp, Artificial Sequence
  5′ UTR-Syn1 Hs
agtctgcggtgggcagcggaggagtcgtgtcgtgcctgagagcgcagctgtgctcctgggcaccgcgcagtccgcccccgcggctcctggccagaccacccctaggaccccctgccccaagtcgca

SEQ ID NO: 23 - DNA, 83 bp, Artificial Sequence
  5′ UTR human CamKIIa
tcagaagccccgggctcgtcagtcaaaccggttctctgtttgcactcggcagcacgggcaggcaagtggtccctaggttcggg

SEQ ID NO: 24 - DNA, 141 bp, Artificial Sequence
  5′ ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 25 - DNA, 168 bp, Artificial Sequence
  5′ ITR second version
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 26 - DNA, 141 bp, Artificial Sequence
  3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 27 - DNA, 168 bp, Artificial Sequence
  3′ ITR second version
tacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 28 - DNA, 4214 bp, Artificial Sequence
  AAV2 hSyn1 SLC6A3 vector construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcaattcagtcgataactataacggtcctaaggtagcgatttaaatacgcgctctcttaaggtagccccgggacgcgtcaattgactacaaaccgagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagccttcgagctagcgtttaaacttaagcttggtaccgagctcggatccactagtccagtgtggtggaattcctcaactcccagtgtgcccatgagtaagagcaaatgctccgtgggactcatgtcttccgtggtggccccggctaaggagcccaatgccgtgggcccgaaggaggtggagctcatccttgtcaaggagcagaacggagtgcagctcaccagctccaccctcaccaacccgcggcagagccccgtggaggcccaggatcgggagacctggggcaagaagatcgactttctcctgtccgtcattggctttgctgtggacctggccaacgtctggcggttcccctacctgtgctacaaaaatggtggcggtgccttcctggtcccctacctgctcttcatggtcattgctgggatgccacttttctacatggagctggccctcggccagttcaacagggaaggggccgctggtgtctggaagatctgccccatactgaaaggtgtgggcttcacggtcatcctcatctcactgtatgtcggcttcttctacaacgtcatcatcgcctgggcgctgcactatctcttctcctccttcaccacggagctcccctggatccactgcaacaactcctggaacagccccaactgctcggatgcccatcctggtgactccagtggagacagctcgggcctcaacgacacttttgggaccacacctgctgccgagtactttgaacgtggcgtgctgcacctccaccagagccatggcatcgacgacctggggcctccgcggtggcagctcacagcctgcctggtgctggtcatcgtgctgctctacttcagcctctggaagggcgtgaagacctcagggaaggtggtatggatcacagccaccatgccatacgtggtcctcactgccctgctcctgcgtggggtcaccctccctggagccatagacggcatcagagcatacctgagcgttgacttctaccggctctgcgaggcgtctgtttggattgacgcggccacccaggtgtgcttctccctgggcgtggggttcggggtgctgatcgccttctccagctacaacaagttcaccaacaactgctacagggacgcgattgtcaccacctccatcaactccctgacgagcttctcctccggcttcgtcgtcttctccttcctggggtacatggcacagaagcacagtgtgcccatcggggacgtggccaaggacgggccagggctgatcttcatcatctacccggaagccatcgccacgctccctctgtcctcagcctgggccgtggtcttcttcatcatgctgctcaccctgggtatcgacagcgccatgggtggtatggagtcagtgatcaccgggctcatcgatgagttccagctgctgcacagacaccgtgagctcttcacgctcttcatcgtcctggcgaccttcctcctgtccctgttctgcgtcaccaacggtggcatctacgtcttcacgctcctggaccattttgcagccggcacgtccatcctctttggagtgctcatcgaagccatcggagtggcctggttctatggtgttgggcagttcagcgacgacatccagcagatgaccgggcagcggcccagcctgtactggcggctgtgctggaagctggtcagcccctgctttctcctgttcgtggtcgtggtcagcattgtgaccttcagacccccccactacggagcctacatcttccccgactgggccaacgcgctgggctgggtcatcgccacatcctccatggccatggtgcccatctatgcggcctacaagttctgcagcctgcctgggtcctttcgagagaaactggcctacgccattgcacccgagaaggaccgtgagctggtggacagaggggaggtgcgccagttcacgctccgccactggctcaaggtgtagagggagcagagacgaagaccccaggaagtcatcctgcaatgggagagacacgaacaaaccaaggaaatctaagtttcgagagaaaggagggcaacttctactcttcaacctctactgaaaacacaaacaacaaagcagaagactcctctcttctgactgtttacacctttccgtgccgggagcgcacctcgccgtgtcttgtgttgctgtaataacgacgtagatctgtgcagcgaggtccaccccgttgttgtcccatccagcacagtggcggccgctcgagtctagagggcccttcgaaggtaagcctatccctaaccctctcctcggtctcgattctacgcgtaccggttaatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgaaacccgctgatcagccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

(19) United States
(12) Patent Application Publication
(10) Pub. No.: US 2024/0115737 A1
(43) Pub. Date: Apr. 11, 2024

(54) GENE THERAPY FOR DOPAMINE TRANSPORTER DEFICIENCY SYNDROME

(71) Applicant: UCL Business Ltd, London (GB)

(72) Inventors: Joanna NG, London (GB); Manju KURIAN, London (GB); Simon WADDINGTON, London (GB); John COUNSELL, London (GB)

(21) Appl. No.: 18/277,023

(22) PCT Filed: Feb. 14, 2022
(86) PCT No.: PCT/GB2022/050396

§ 371 Date: Aug. 11, 2023

(30) Foreign Application Priority Data
Feb. 12, 2021 (GB) 2101958.3

(51) Int. Cl. A61K 48/00; A01K 67/0276; A61K 9/00; A61P 25/28; C07K 14/705; C12N 15/86

(52) U.S. Cl. CPC A61K 48/0058; A01K 67/0276; A61K 9/0019; A61K 9/0085; A61K 48/0075; A61K 48/0083; A61P 25/28; C07K 14/70571; C12N 15/86; C12N 2750/14143

(57) ABSTRACT

There is described a vector for treating Dopamine Transporter Deficiency Syndrome, the vector comprising a promoter operably linked to a human SLC6A3 gene, wherein the promoter is selected from a human synapsin 1 promoter, a CAG promoter, a CMV promoter, a CAMKII promoter, a beta-actin promoter, and a human EF1-alpha promoter. Also described are methods and uses involving the vector for treating Dopamine Transporter Deficiency Syndrome.

### FIELD OF THE INVENTION

The present invention relates to gene therapy vectors for the treatment of Dopamine Transporter Deficiency Syndrome.

### BACKGROUND TO THE INVENTION

Dopamine Transporter Deficiency Syndrome (DTDS) is an infantile-onset parkinsonian disorder caused by recessive loss-of-function mutations in SLC6A3 gene, encoding the dopamine transporter (DAT). DAT is highly expressed in pre-synaptic midbrain dopaminergic neurons, where it re-uptakes released dopamine (DA) from the synaptic cleft. It is a key regulator of the amplitude and duration of dopaminergic transmission.

DTDS is an ultra-rare disease and to the inventors' knowledge, there are 45 genetically proven cases, of which 28 are published; a further 17 are currently unpublished cases, referred to the inventors' centre between 2015 and 2020. Given that many reported patients have been misdiagnosed with cerebral palsy, the true incidence of DTDS is likely to be higher. Patients with DTDS present with a progressive movement disorder characterized initially by infantile-onset hyperkinesia, with features of dystonia, choreoathetosis, ballismus, orolingual dyskinesia and recurrent oculogyric crises. Life-threatening status dystonicus is commonly reported. Over time, children develop severe parkinsonism with akinesia, rigidity, tremor and hypomimia in late childhood or early adolescence. Affected patients show characteristic findings on cerebrospinal fluid (CSF) neurotransmitter analysis, with raised levels of the dopamine metabolite, homovanillic acid (HVA) and normal levels of the serotonin metabolite 5-hydroxyindoleacetic acid (5-HIAA), leading to a pathologically increased CSF HVA:HIAA ratio. The relentless disease course and lack of effective therapies frequently leads to premature death in the first or second decade of life, usually secondary to respiratory complications.

Very little is known about the cellular progression of DTDS in the central nervous system of affected patients. Progressive changes on single-photon emission computed tomography (SPECT) imaging with ioflupane (1123) (also known as DaTscan) have been reported in a patient with atypical DTDS over an 8 year interval. Whilst this is suggestive of progressive nigrostriatal neurodegeneration, the absence of post-mortem data from DTDS patients has negated pathological confirmation of this clinical observation. To date, limited understanding of the cellular mechanisms underpinning DTDS disease pathogenesis has hindered the development of effective disease-modifying or curative therapies.

Pharmacochaparones (biproprion, ibogaine, noribogaine and pifithrin-μ) have been shown to restore DAT cell trafficking to improve function in vitro and in a Drosophila model, but the response to these pharmacochaparones is dependent on the particular mutation and therefore not suited to all patients. Further, they would be ineffective for patients with protein-truncating variants where there is predicted absence of DAT protein.

Previously used gene therapy approaches for DTDS are not clinically applicable. P. Illiano et al. (Scientific Reports 7, 46280 (2017)) used dual vector technology to restore DAT gene function in the DAT knockout murine model. Two separate AAV vectors are delivered: the first, containing the mouse DAT gene, only yields DAT expression when co-delivered with a second vector, expressing Cre recombinase delivered by stereotactic injection. The viral vector technology utilized is not translational and would not be permitted in a clinical trial, for example, due to potential neurotoxicity associated with Cre recombinase expression in the brain.

Chen et al. (The Journal of Neuroscience, 28(2):425-433 (2008)) describes the generation of transgenic mice which express DAT in non-dopaminergic neurons. These mice developed motor dysfunction and progressive striatal neurodegeneration. This suggests that ectopic expression of DAT using a gene therapy approach would cause similar problems.

Therefore, there is a need for a gene therapy approach suitable for the clinical treatment of DTDS.

### SUMMARY OF THE INVENTION

In a first aspect of the invention, there is provided a vector for treating Dopamine Transporter Deficiency Syndrome (DTDS), wherein the vector comprises a promoter operably linked to a human SLC6A3 gene, wherein the promoter is selected from a human synapsin 1 promoter, a CAG promoter, a cytomegalovirus (CMV) promoter, a CAMKII promoter, a beta-actin promoter and a human EF1-α promoter.

DTDS is caused by mutations in the SLC6A3 gene, which results in impaired or eliminated function of the dopamine transporter (DAT). DAT is highly expressed in pre-synaptic midbrain dopaminergic neurons, where it re-uptakes released dopamine (DA) from the synaptic cleft. The introduction of the SLC6A3 gene which expresses the dopamine transporter protein compensates for the mutated gene and ameliorates the effects of DTDS. The vector defined above can provide tissue-specific transduction in neurons. Therefore, this allows the vector to preferentially target the dopaminergic neurons to address the impaired function of DAT.

The vector may be any vector. For example, the vector may be an adeno-associated viral (AAV) vector, an adenoviral vector, a retroviral vector (such as a lentiviral vector), an alphaviral vector, a flaviviral vector, a herpes simplex viral vector, a rhabdoviral vector, a measles viral vector, a pox viral vector, a newcastle disease viral vector, a coxsackieviral vector, or a non-viral vector, such as a polyvalent cation, lipid nanoparticle, chitosan nanoparticle, PLGA dendrimer or other conjugate allowing cellular uptake. Preferably, the vector is a viral vector. More preferably, the vector is an AAV vector or a lentiviral vector.

In some embodiments, the vector is an adeno-associated viral (AAV) vector. The adeno-associated viral vector may be a recombinant adeno-associated viral (rAAV) vector. AAV is a member of the family Parvoviridae which is described in Kenneth I. Berns, “Parvoviridae: The Viruses and Their Replication,” Chapter 69 in Fields Virology (3d Ed. 1996). AAV vectors are also described in “Adeno-Associated Virus Vectors. Design and Delivery”, Editor: Castle, Michael J. (ISBN 978-1-4939-9139-6) and “Adeno-Associated Virus (AAV) Vectors in Gene Therapy”, Editors: Berns, Kenneth I. and Giraud, Catherine (ISBN 978-3-642-80207-2).

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, −2 and −3) form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. Following wild type (wt) AAV infection in mammalian cells the Rep genes (i.e. encoding Rep78 and Rep52 proteins) are expressed from the P5 promoter and the P19 promoter, respectively, and both Rep proteins have a function in the replication of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

In an AAV suitable for use as a gene therapy vector, the vector genome typically comprises a nucleic acid (e.g. a human SLC6A3 gene) to be packaged for delivery to a target cell. According to a particular embodiment, the heterologous nucleotide sequence is located between the viral ITRs at either end of the vector genome. In further preferred embodiments, the AAV cap genes and AAV rep genes are deleted from the template genome (and thus from the virion DNA produced therefrom). This configuration maximizes the size of the nucleic acid sequence(s) that can be carried by the AAV capsid. Following transfection into a host cell that has been co-transfected with a plasmid or plasmids encoding and expressing rep and cap genes, or a host cell that has been stably engineered to express rep and cap genes, the AAV vectors can be replicated and packaged into infectious viral particles.

According to this particular embodiment, the nucleic acid is located between the viral ITRs at either end of the substrate. It is possible for an AAV genome to function with only one ITR. Thus, in a gene therapy vector based on an AAV, the vector genome is typically flanked by at least one ITR, but, more typically, by two AAV ITRs (generally with one either side of the vector genome, i.e. one at the 5′ end and one at the 3′ end). There may be intervening sequences between the nucleic acid in the vector genome and one or more of the ITRs. The ITR nucleotide sequences may be selected from SEQ ID NOs: 24-27.

Generally, the human SLC6A3 gene (i.e. the nucleotide sequence encoding a functional DAT protein (for expression in the mammalian cell)) will be incorporated into a parvoviral genome located between two regular ITRs or located on either side of an ITR engineered with two D regions.

AAV sequences that may be used in the present invention for the production of AAV gene therapy vectors can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent albeit with certain differences in tropism, and replicate and assemble by practically identical mechanisms. AAV serotype 1, 2, 3, 4, 5, 6, 7, 8 or 9 may be used in the present invention. The sequences from the AAV serotypes may be mutated or engineered when being used in the production of gene therapy vectors. In some embodiments, non-human primate AAV serotypes may be used such as those described in WO 03/042397; Gao et al., PNAS, vol. 99, no. 18, pp. 11854-11859 (2002); Castle et al., Methods Mol Biol, 1382: 133-149 (2016); Klein et al. Mol Ther., 16(1): 89-96 (2008); Selot et al., Frontiers in Pharmacology, Volume 8, Article 441 (July 2017), Tanguy et al., Frontiers in Molecular Neuroscience, Volume 8, Article 36 (July 2015), all of which are incorporated herein by reference. In particular, non-human primate AAV serotypes designated as rh serotypes can be used such as AAVrh10 and AAVrh43. Other suitable vectors include AAV-PHP.A and AAVPHP.B (Nature Biotechnology 34, 204-209 (2016)), AAV9.47 (Hum Gene Ther. 2016 July; 27(7):497-508), AAV-B1 (Mol. Ther. 24, 1247-1257), AAV8 (Y733F) (Mol Ther 2009; 17: 463-471) and AAV2-TT (described in WO2015/121501).

Preferably, the AAV ITR sequences for use in the context of the present invention are derived from AAV1, AAV2, AAV4 and/or AAV6. Likewise, the Rep (Rep78 and Rep52) coding sequences are preferably derived from AAV1, AAV2, AAV4 and/or AAV6. The sequences coding for the VP1, VP2, and VP3 capsid proteins for use in the context of the present invention may however be taken from any of the known 42 serotypes, more preferably from AAV1, AAV2, AAV3, AAV4, AAV5, AAV6, AAV7, AAV8 or AAV9 or newly developed AAV-like particles obtained by e.g. capsid shuffling techniques and AAV capsid libraries. Hybrid serotypes can also be used such as those described in Grimm et al., J. Virol. 82, 5887-5911 (2008) and Jang et al. Frontiers in Cellular Neuroscience, Volume 12, Article 157 (June 2018), both of which are incorporated herein by reference.

AAV Rep and ITR sequences are particularly conserved among most serotypes. Moreover, the Rep sequences and ITRs of many AAV serotypes are known to efficiently cross-complement (i.e., functionally substitute) corresponding sequences from other serotypes in production of AAV particles in mammalian cells.

The AAV VP proteins are known to determine the cellular tropism of the AAV virion. The VP protein-encoding sequences are significantly less conserved than Rep proteins and genes among different AAV serotypes. The ability of Rep and ITR sequences to cross-complement corresponding sequences of other serotypes allows for the production of pseudotyped AAV particles comprising the capsid proteins of a serotype (e.g., AAV1, or 8) and the Rep and/or ITR sequences of another AAV serotype (e.g., AAV2). Such pseudotyped rAAV particles are a part of the present invention.

Modified “AAV” sequences also can be used in the context of the present invention, e.g. for the production of AAV gene therapy vectors. Such modified sequences e.g. include sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAV5, AAV6, AAV7, AAV8 or AAV9 ITR, Rep, or VP can be used in place of wild-type AAV ITR, Rep, or VP sequences.

The viral capsid used in the invention may be from any parvovirus, either an autonomous parvovirus or dependovirus, as described above. Preferably, the viral capsid is an AAV capsid (e. g., AAV1, AAV2, AAV3, AAV4, AAV5 or AAV6 capsid). The choice of parvovirus capsid may be based on a number of considerations as known in the art, e.g., the target cell type, the desired level of expression, the nature of the heterologous nucleotide sequence to be expressed, issues related to viral production, and the like.

A parvovirus gene therapy vector prepared according to the invention may be a “hybrid” particle in which the viral TRs and viral capsid are from different parvoviruses. Preferably, the viral TRs and capsid are from different serotypes of AAV. Likewise, the parvovirus may have a “chimeric” capsid (e. g., containing sequences from different parvoviruses, preferably different AAV serotypes) or a “targeted” capsid (e. g., a directed tropism).

In the context of the disclosure “at least one parvoviral ITR nucleotide sequence” is understood to mean a palindromic sequence, comprising mostly complementary, symmetrically arranged sequences also referred to as “A,” “B,” and “C” regions. The ITR functions as an origin of replication, a site having a “cis” role in replication, i.e., being a recognition site for trans-acting replication proteins such as e.g. Rep 78 (or Rep68) which recognize the palindrome and specific sequences internal to the palindrome. One exception to the symmetry of the ITR sequence is the “D” region of the ITR. It is unique (not having a complement within one ITR). Nicking of single-stranded DNA occurs at the junction between the A and D regions. It is the region where new DNA synthesis initiates. The D region normally sits to one side of the palindrome and provides directionality to the nucleic acid replication step. A parvovirus replicating in a mammalian cell typically has two ITR sequences. It is, however, possible to engineer an ITR so that binding sites are on both strands of the A regions and D regions are located symmetrically, one on each side of the palindrome. On a double-stranded circular DNA template (e.g., a plasmid), the Rep78- or Rep68-assisted nucleic acid replication then proceeds in both directions and a single ITR suffices for parvoviral replication of a circular vector. Thus, one ITR nucleotide sequence can be used in the context of the present invention. Preferably, however, two or another even number of regular ITRs are used. Most preferably, two ITR sequences are used. A preferred parvoviral ITR is an AAV ITR. For safety reasons it may be desirable to construct a parvoviral (AAV) vector that is unable to further propagate after initial introduction into a cell. Such a safety mechanism for limiting undesirable vector propagation in a recipient may be provided by using AAV with a chimeric ITR.

Those skilled in the art will appreciate that the viral Rep protein(s) used for producing an AAV vector of the invention may be selected with consideration for the source of the viral ITRs. For example, the AAV5 ITR typically interacts more efficiently with the AAV5 Rep protein, although it is not necessary that the serotype of ITR and Rep protein(s) are matched.

The ITR(s) used in the invention are typically functional, i.e. they may be fully resolvable and are preferably AAV sequences. Resolvable AAV ITRs according to the present invention need not have a wild-type ITR sequence (e. g., a wild-type sequence may be altered by insertion, deletion, truncation or missense mutations), as long as the ITR mediates the desired functions, e. g., virus packaging, integration, and/or provirus rescue, and the like.

Preferably, the vector is an AAV2 vector. This includes AAV vectors which have been pseudotyped with the capsid proteins from AAV2, i.e. where the genome of other AAV serotypes has been packaged in the capsid proteins of AAV2. Such pseudotyped vectors would be well known to those skilled in the art. Further, the capsid proteins of the vector can be hybrid, mixed or chimeric capsids in which the capsid proteins from other AAV serotypes are used with the AAV2 capsid proteins. Such hybrid/mixed/chimeric vectors would be well known to those skilled in the art, examples of which include a hybrid capsid of AAV1 and AAV2. In some embodiments, the vector is an AAV2 vector which is not a pseudotyped or chimeric vector. The use of the AAV2 capsid provides targeted transduction of the dopaminergic neurons of the brain.

In other embodiments, the vector is a lentiviral vector. The lentivirus group can be split into “primate” and “non-primate”. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

Details on the genomic structure of some lentiviruses may be found in the art. By way of example, details on HIV and EIAV may be found from the NCBI Genbank database (i.e. Genome Accession Nos. AF033819 and AF033820 respectively). Details of HIV variants may also be found at http://hiv.lanl.gov. Details of EIAV variants may be found through http://www.ncbi.nlm.nih.gov. Exemplary lentiviral vectors are described in Trends in Molecular Medicine, April 2016, Vol. 22, No. 4 and Ther Deliv. 2010 October; 1(4): 517-534.

During the process of infection, a retrovirus initially attaches to a specific cell surface receptor. On entry into the susceptible host cell, the retroviral RNA genome is then copied to DNA by the virally encoded reverse transcriptase which is carried inside the parent virus. This DNA is transported to the host cell nucleus where it subsequently integrates into the host genome. At this stage, it is typically referred to as the provirus. The provirus is stable in the host chromosome during cell division and is transcribed like other cellular genes. The provirus encodes the proteins and other factors required to make more virus, which can leave the cell by a process sometimes called “budding”.

Each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is at the boundary between U3 and R in the left hand side LTR and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins. Some retroviruses have any one or more of the following genes that code for proteins that are involved in the regulation of gene expression: tat, rev, tax and rex.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction leads ultimately to infection by fusion of the viral membrane with the cell membrane.

Retroviruses may also contain “additional” genes which code for proteins other than gag, pol and env. Examples of additional genes include in HIV, one or more of vif, vpr, vpx, vpu, tat, rev and nef. EIAV has (amongst others) the additional gene S2.

Proteins encoded by additional genes serve various functions, some of which may be duplicative of a function provided by a cellular protein. In EIAV, for example, tat acts as a transcriptional activator of the viral LTR. It binds to a stable, stem-loop RNA secondary structure referred to as TAR. Rev regulates and co-ordinates the expression of viral genes through rev-response elements (RRE). The mechanisms of action of these two proteins are thought to be broadly similar to the analogous mechanisms in the primate viruses. The function of S2 is unknown. In addition, an EIAV protein, Ttm, has been identified that is encoded by the first exon of tat spliced to the env coding sequence at the start of the transmembrane protein.

The lentiviral vector of the present disclosure may be a recombinant lentiviral vector. As used herein, the term “recombinant lentiviral vector” (RLV) refers to a vector with sufficient genetic information to allow packaging of an RNA genome, in the presence of packaging components, into a viral particle capable of infecting and transducing a target cell. Infection and transduction of a target cell includes reverse transcription and integration into the target cell genome. The RLV carries non-viral coding sequences which are to be delivered by the vector to the target cell. An RLV is incapable of independent replication to produce infectious retroviral particles within the final target cell. Usually the RLV lacks a functional gag-pol and/or env gene and/or other genes essential for replication.

Preferably the recombinant lentiviral vector (RLV) of the present disclosure has a minimal viral genome. As used herein, the term “minimal viral genome” means that the viral vector has been manipulated so as to remove the non-essential elements and to retain the essential elements in order to provide the required functionality to infect, transduce and deliver a nucleotide sequence of interest to a target host cell.

A minimal lentiviral genome for use in the present disclosure will therefore comprise (5′) R—U5—one or more first nucleotide sequences—(regulatory element—NOI)n—U3-R (3′).

However, the plasmid vector used to produce the lentiviral genome within a host cell/packaging cell will also include transcriptional regulatory control sequences operably linked to the lentiviral genome to direct transcription of the genome in a host cell/packaging cell. These regulatory sequences may be the natural sequences associated with the transcribed retroviral sequence, i.e. the 5′ U3 region, or they may be a heterologous promoter such as another viral promoter, for example the CMV promoter. Some lentiviral genomes require additional sequences for efficient virus production. For example, in the case of HIV, rev and RRE sequence are preferably included.

The vector may have at least one of the following: the ATG motifs of the gag packaging signal of the wild type viral vector are ATTG motifs; the distance between the R regions of the viral vector is substantially the same as that in the wild type viral vector; the 3′ U3 region of the viral vector includes sequence from an MLV U3 region; and a nucleotide sequence operably linked to the viral LTR and wherein said nucleotide sequence is upstream of an internal promoter and wherein said nucleotide sequence preferably encodes a polypeptide or fragment thereof.

In a preferred embodiment, the system is based on a so-called “minimal” system in which some or all of the additional genes have be removed.

Preferably the lentiviral vector is a self-inactivating vector. In other words the viral promoter is a self-inactivating LTR.

As known in the art, self-inactivating retroviral vectors have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus. However, any promoter(s) internal to the LTRs in such vectors will still be transcriptionally active. This strategy has been employed to eliminate effects of the enhancers and promoters in the viral LTRs on transcription from internally placed genes.

In one embodiment, the lentiviral vector is derived from a non-primate lentivirus. The non-primate lentivirus may be any member of the family of lentiviridae which does not naturally infect a primate and may include a feline immunodeficiency virus (FIV), a bovine immunodeficiency virus (BIV), a caprine arthritis encephalitis virus (CAEV), a Maedi visna virus (MW) or an equine infectious anaemia virus (EIAV). Non-primate lentiviral-based vectors do not introduce HIV proteins into individuals.

The “promoter” refers to the polynucleotide sequence that is capable of promoting initiation of RNA transcription of a polynucleotide from the transcription initiation site. The promoter contained in the vector is operably linked to the human SLC6A3 gene so that the promoter directs expression of the human SLC6A3 gene in the transduced cells. This means that the SLC6A3 gene is suitably positioned and oriented relative to the promoter for transcription of the SLC6A3 gene to be initiated from the promoter. Therefore, the SLC6A3 gene should be in the same orientation as the promoter so that transcription which is initiated at the promoter produces a functional RNA molecule encoding the DAT protein. The promoter results in expression of human SLC6A3 in the transduced neurons so that the expressed DAT protein restores dopamine neurotransmission and ameliorates the pathologies associated with DTDS.

The promoter is selected from a human synapsin 1 promoter, a CAG promoter, a cytomegalovirus (CMV) promoter, a CAMKII promoter, a beta-actin promoter and a human eukaryotic translation elongation factor 1 α (EF1-α) promoter.

A human synapsin 1 promoter includes any promoter comprising a functional portion of the human synapsin 1 gene. For example, the promoter may be a human synapsin 1 (hSYN1) promoter, a human synapsin 1 with 5′ extension promoter (e.g. a nucleotide sequence according to SEQ ID NO: 2), a human synapsin 1 with 3′ extension promoter (e.g. a nucleotide sequence according to SEQ ID NO: 3) an eSYN promoter (e.g. a nucleotide sequence according to SEQ ID NO: 4) or a truncated human synapsin 1 promoter (e.g. a nucleotide sequence according to SEQ ID NO: 1). The hSYN1 promoter is well known to those skilled in the art. The promoter comprising a functional portion of the human synapsin 1 gene may additionally comprise an enhancer element, which may be any enhancer element. For example, the eSYN promoter is a hybrid promoter containing the human synapsin 1 promoter and a CMV enhancer.

A beta-actin promoter includes any functional promoter comprising a functional portion of the beta-actin gene. The beta-actin promoter may be a human beta-actin promoter (e.g. a nucleotide sequence according to SEQ ID NO: 5) or a chicken beta-actin promoter (e.g. a nucleotide sequence according to SEQ ID NO: 6). The promoter comprising a functional portion of the beta-actin gene may additionally comprise an enhancer element, which may be any enhancer element. For example, the promoter may be a CAG promoter (e.g. a nucleotide sequence according to SEQ ID NO: 7), which comprises the CMV early enhancer element, the promoter, first exon and first intron of chicken beta-actin gene and the splice acceptor or the rabbit beta-globin gene.

A CMV promoter may be a human CMV major immediate early promoter (e.g. a nucleotide sequence according to SEQ ID NO: 8 or 9) or a super CMV promoter.

A CAMKII promoter may be an α-CAMKII promoter (e.g. a nucleotide sequence according to SEQ ID NO: 10).

A human EF1-α promoter may have a nucleotide sequence according to SEQ ID NO: 11.

In certain embodiments, the promoter is a neuron-specific promoter. For example, neuron-specific promoters include human synapsin 1 promoters and CAMKII promoters. In particular embodiments, the promoter is a neuron-specific promoter selected from a human synapsin 1 promoter and a CAMKII promoter. In various embodiments, the promoter is a neuron-specific promoter selected from an hSYN1 promoter, an hSYN1 with 5′ extension promoter, an hSYN1 with 3′ extension promoter, an eSYN promoter, a truncated hSYN1 promoter and an α-CAMKII promoter. In some embodiments, the promoter is a human synapsin 1 promoter. In particular embodiments, the promoter is an hSYN1 promoter, an hSYN1 with 5′ extension promoter, an hSYN1 with 3′ extension promoter, an eSYN promoter or a truncated hSYN1 promoter. In certain embodiments, the promoter is a dopaminergic neuron-specific promoter.

The inventors have surprisingly found that the gene therapy vector is efficacious with a range of different promoters. For example, the promoter does not need to be a dopaminergic neuron-specific promoter for efficient and selective and safe transduction of dopaminergic neurons. This was unexpected, as one skilled in the art would have presumed that a dopaminergic neuron-specific promoter would be required for selective transduction of dopaminergic neurons without toxic off-target effects. Further, the fact that it has been previously shown that expression of DAT in non-dopaminergic neurons causes motor dysfunction and progressive striatal neurodegeneration in mice (see Chen et al. (2008) referred to in the background section) would cause a skilled person to expect such gene therapy vectors to cause similar problems. However, the results detailed below demonstrate, unexpectedly, that the vectors of the invention do not lead to neurotoxicity in either the short or long term.

The vector causes the direct expression of DAT protein from the vector. More precisely, as a result of the promoter being operably linked to the human SLC6A3 gene, transcription initiated at the promoter produces an RNA molecule encoding the DAT protein and this RNA molecule is then translated to produce the DAT protein which can then ameliorates the pathologies associated with DTDS. In view of this, it is not necessary to genetically manipulate the SLC6A3 gene in order to cause expression. For example, the vector does not require use of a Cre Recombinase system and so does not comprise a lox site which is needed for the functioning of the Cre recombinase system. Preferably, the vector does not comprise any lox site, including loxP and variants such as lox2272, lox511, lox5171, lox71, lox66 and loxN. Preferably, the vector does not comprise an FRT (flippase recognition target) site. This has the advantage that the invention does not rely on the Cre-Lox recombination system (or the FLP-FRT recombination system) which has associated neurotoxicity.

The vector comprises a human SLC6A3 gene. The SLC6A3 gene encodes the DAT protein. The human SLC6A3 gene encodes a functional DAT protein. This means that the protein, when expressed, has the same function and activity as the wild type human protein. This could easily be determined by one skilled in the art. The protein encoded by the human SLC6A3 gene may be the wild type human DAT protein. The wild type human sequence of the DAT protein is well known to those skilled in the art. For example, it can be found on the publically accessible databases of the National Center for Biotechnology Information. Further, the nucleotide sequences which encode this protein (and which would be contained in the vector) could readily be found or determined by a person skilled in the art, for example, using the genetic code which correlates particular nucleotide codons with particular amino acids.

The DAT protein encoded by the human SLC6A3 gene preferably does not contain additional amino acids that are not found in the wild type protein. Any additional amino acids could interfere in the normal functioning of the protein. For example, it is preferred that the DAT protein does not comprise a fluorescent protein such as green fluorescent protein (GFP) or mCherry, or tags such as a FLAG-tag or a polyhistidine-tag.

In particular embodiments, the human SLC6A3 gene has the nucleotide sequence of SEQ ID NO: 12 or has at least 70% sequence identity thereto, and encodes a functional DAT protein. In some embodiments, the human SLC6A3 gene has the nucleotide sequence of SEQ ID NO: 12 or has at least 72% sequence identity thereto, and encodes a functional DAT protein. In a number of embodiments, the human SLC6A3 gene has the nucleotide sequence of SEQ ID NO: 12 or has at least 74% sequence identity thereto, and encodes a functional DAT protein. In other embodiments, the human SLC6A3 gene has the nucleotide sequence of SEQ ID NO: 12 or has at least 76% sequence identity thereto, and encodes a functional DAT protein. In various embodiments, the human SLC6A3 gene has the nucleotide sequence of SEQ ID NO: 12 or has at least 78% sequence identity thereto, and encodes a functional DAT protein. In particular embodiments, the human SLC6A3 gene has the nucleotide sequence of SEQ ID NO: 12 or has at least 80% sequence identity thereto, and encodes a functional DAT protein. In some embodiments, the human SLC6A3 gene has the nucleotide sequence of SEQ ID NO: 12 or has at least 82% sequence identity thereto, and encodes a functional DAT protein. In a number of embodiments, the human SLC6A3 gene has the nucleotide sequence of SEQ ID NO: 12 or has at least 84% sequence identity thereto, and encodes a functional DAT protein. In other embodiments, the human SLC6A3 gene has the nucleotide sequence of SEQ ID NO: 12 or has at least 86% sequence identity thereto, and encodes a functional DAT protein. In various embodiments, the human SLC6A3 gene has the nucleotide sequence of SEQ ID NO: 12 or has at least 88% sequence identity thereto, and encodes a functional DAT protein. In particular embodiments, the human SLC6A3 gene has the nucleotide sequence of SEQ ID NO: 12 or has at least 90% sequence identity thereto, and encodes a functional DAT protein. In some embodiments, the human SLC6A3 gene has the nucleotide sequence of SEQ ID NO: 12 or has at least 92% sequence identity thereto, and encodes a functional DAT protein. In a number of embodiments, the human SLC6A3 gene has the nucleotide sequence of SEQ ID NO: 12 or has at least 94% sequence identity thereto, and encodes a functional DAT protein. In other embodiments, the human SLC6A3 gene has the nucleotide sequence of SEQ ID NO: 12 or has at least 95% sequence identity thereto, and encodes a functional DAT protein. In various embodiments, the human SLC6A3 gene has the nucleotide sequence of SEQ ID NO: 12 or has at least 96% sequence identity thereto, and encodes a functional DAT protein. In various embodiments, the human SLC6A3 gene has the nucleotide sequence of SEQ ID NO: 12 or has at least 97% sequence identity thereto, and encodes a functional DAT protein. In particular embodiments, the human SLC6A3 gene has the nucleotide sequence of SEQ ID NO: 12 or has at least 98% sequence identity thereto, and encodes a functional DAT protein. In some embodiments, the human SLC6A3 gene has the nucleotide sequence of SEQ ID NO: 12 or has at least 99% sequence identity thereto, and encodes a functional DAT protein. In a number of embodiments, the human SLC6A3 gene has the nucleotide sequence of SEQ ID NO: 12.

In the embodiments above, the nucleotide sequence of the human SLC6A3 gene may be codon optimised to maximise expression of the protein. In codon optimisation, the amino acid sequence of the encoded protein remains the same so it will still be functional. It is simply the nucleotide sequence that is modified.

In various embodiments, the human SLC6A3 gene encodes a functional DAT protein having the amino acid sequence of SEQ ID NO: 13 or has at least 80% sequence identity thereto. In some embodiments, the human SLC6A3 gene encodes a functional DAT protein having the amino acid sequence of SEQ ID NO: 13 or has at least 85% sequence identity thereto. In other embodiments, the human SLC6A3 gene encodes a functional DAT protein having the amino acid sequence of SEQ ID NO: 13 or has at least 90% sequence identity thereto. In a number of embodiments, the human SLC6A3 gene encodes a functional DAT protein having the amino acid sequence of SEQ ID NO: 13 or has at least 95% sequence identity thereto. In particular embodiments, the human SLC6A3 gene encodes a functional DAT protein having the amino acid sequence of SEQ ID NO: 13.

In the description above, the term “identity” is used to refer to the similarity of two sequences. For the purpose of this invention, it is defined here that in order to determine the percent identity of two sequences, the sequences are aligned for optimal comparison purposes (e.g., gaps can be introduced in the sequence of a first sequence for optimal alignment with a second amino or nucleic acid sequence). The nucleotide/amino acid residues at each position are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Generally, the two sequences are the same length. A sequence comparison is typically carried out over the entire length of the two sequences being compared.

The skilled person will be aware of the fact that several different computer programs are available to determine the identity between two sequences. For instance, a comparison of sequences and determination of percent identity between two sequences can be accomplished using a mathematical algorithm. In a preferred embodiment, the percent identity between two nucleic acid sequences is determined using the sequence alignment software Clone Manager 9 (Sci-Ed software—www.scied.com) using global DNA alignment; parameters: both strands; scoring matrix: linear (mismatch 2, OpenGap 4, ExtGap 1).

Alternatively, the percent identity between two amino acid or nucleic acid sequences can be determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6. A further method to assess the percent identity between two amino acid or nucleic acid sequences can be to use the BLAST sequence comparison tool available on the National Center for Biotechnology Information (NCBI) website (www.blast.ncbi.nlm.nih.gov), for example using BLASTn for nucleotide sequences or BLASTp for amino acid sequences using the default parameters.

The vector may additionally comprise one or more regulatory elements. The one or more regulatory elements may be selected from enhancers, introns, polyadenylation sequences, 2A peptide encoding sequences, and transcript-stabilising elements.

In various embodiments, the vector additionally comprises one or more enhancers. The enhancer(s) may be any enhancer(s) known in the art. For example, the enhancer(s) may be a CMV enhancer, a GAPDH enhancer, a β-actin enhancer, an EF1-α enhancer and/or a TPL-eMLP adenovirus derived enhancer. In certain embodiments, the vector comprises a CMV enhancer (e.g. having the nucleotide sequence according to SEQ ID NO: 14). In certain embodiments, the vector comprises a TPL-eMLP adenovirus derived enhancer (e.g. having the nucleotide sequence according to SEQ ID NO: 15). The one or more enhancers may be located downstream or upstream of the promoter.

In various embodiments, the vector additionally comprises one or more introns and/or exons. In particular embodiments, the intron/exon is a β-globin intron sequence, a β-actin exon/intron sequence, a synthetic intron sequence, an EF1-α intron/exon sequence, an EF1-α intron sequence, a β-actin exon/intron sequence in combination with a β-globin intron sequence and a CMV IE exon sequence. For example, exemplary intron/exon sequences are given as SEQ ID NOs: 16-21. The one or more introns and/or exons may be located downstream of the promoter.

In various embodiments, the vector additionally comprises a polyadenylation (polyA) sequence. The polyA sequence may be a rabbit globin polyA sequence, a human growth hormone polyA sequence, a bovine growth hormone polyA sequence, a PGK polyA sequence, an SV40 polyA sequence, or a TK polyA sequence. In some embodiments, the polyA sequence may be a bovine growth hormone polyadenylation sequence.

In various embodiments, the vector additionally comprises one or more posttranscriptional regulatory elements. The posttranscriptional regulatory element(s) may be a Woodchuck hepatitis virus posttranscriptional regulatory element (WPRE) and/or a hepatitis B posttranscriptional regulatory element (HPRE).

In various embodiments, the vector additionally comprises one or more transcript stabilising elements. The transcript stabilising element(s) may be a scaffold-attachment region, a 5′ untranslated region (UTR), and/or a 3′ UTR. In particular embodiments, the vector comprises a 5′ UTR and a 3′ UTR.

In some embodiments, the vector additionally comprises a 5′ UTR polynucleotide sequence, for example, according to SEQ ID NO: 22 or 23. In various embodiments, the vector comprises a 5′ UTR polynucleotide sequence with at least 100%, 99%, 98%, 97%, 96%, 95%, 94%, 93%, 92%, 91%, 90%, 85%, 80% or 75% sequence identity to SEQ ID NO: 22 or 23.

In one particular embodiment, the vector is an AAV vector or a lentiviral vector, wherein the vector comprises a promoter operably linked to a human SLC6A3 gene, wherein the promoter is selected from a human synapsin 1 promoter, a CAG promoter, a cytomegalovirus (CMV) promoter, a CAMKII promoter, a beta-actin promoter and a eukaryotic translation elongation factor 1 α promoter.

In one embodiment, the vector is an AAV vector comprising a promoter operably linked to a human SLC6A3 gene, wherein the promoter is selected from a human synapsin 1 promoter, a CAG promoter, a cytomegalovirus (CMV) promoter, a CAMKII promoter, a beta-actin promoter and a eukaryotic translation elongation factor 1 α promoter.

In one embodiment, the vector is an AAV2 vector comprising a promoter operably linked to a human SLC6A3 gene, wherein the promoter is selected from a human synapsin 1 promoter, a CAG promoter, a cytomegalovirus (CMV) promoter, a CAMKII promoter, a beta-actin promoter and a eukaryotic translation elongation factor 1 α promoter.

In one embodiment, the vector is an AAV2 vector comprising a neuron-specific promoter operably linked to a human SLC6A3 gene.

In one embodiment, the vector is an AAV2 vector comprising a promoter operably linked to a human SLC6A3 gene, wherein the promoter is a human synapsin 1 promoter or a CAMKII promoter.

In one embodiment, the vector is an AAV2 vector comprising a human synapsin 1 promoter operably linked to a human SLC6A3 gene.

In one embodiment, the vector is an AAV2 vector comprising a human synapsin 1 promoter operably linked to a human SLC6A3 gene, wherein the vector does not comprise a loxP site.

In some embodiments, the vector has the nucleotide sequence according to SEQ ID NO: 28, or has at least 70%, 75%, 80%, 85%, 90%, 91%, 92%, 93%, 94%, 95%, 96%, 97%, 98%, or 99% sequence identity thereto.

In one aspect, the invention provides a pharmaceutical composition comprising a vector as described above and one or more pharmaceutically acceptable excipients. The one or more excipients include carriers, diluents and/or other medicinal agents, pharmaceutical agents or adjuvants, etc.

Acceptable excipients for therapeutic use are well known in the pharmaceutical art, and are described, for example, in Remington's Pharmaceutical Sciences, Mack Publishing Co. (A. R. Gennaro edit. 1985). The choice of pharmaceutical excipient can be selected with regard to the intended route of administration and standard pharmaceutical practice. The pharmaceutical compositions may comprise as, or in addition to, the excipient, any suitable binder, lubricant, suspending agent, coating agent or solubilising agent.

Preservatives, stabilizers and dyes may be provided in the pharmaceutical composition. Examples of preservatives include sodium benzoate, sorbic acid and esters of p-hydroxybenzoic acid. Antioxidants and suspending agents may be also used.

The pharmaceutical composition may also comprise tolerance-promoting adjuvants and/or tolerance promoting cells. Tolerance promoting adjuvants include IL-10, recombinant cholera toxin B-subunit (rCTB), ligands for Toll-like receptor 2, as well as biologics and monoclonal antibodies that modulate immune responses, such as anti-CD3 and co-stimulation blockers, which may be co-administered with the peptide. Tolerance promoting cells include immature dendritic cells and dendritic cells treated with vitamin D3, (1alpha,25-dihydroxy vitamin D3) or its analogues.

For purposes of administration, e.g., by injection, various solutions can be employed, such as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as Pluronic™ F-68 at 0.001% or 0.01%. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include but are not limited to sterile aqueous solutions or dispersions and sterile powders for the extemporaneous preparation of sterile injectable solutions or dispersions. In all cases the form is sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions may be prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

The invention also provides a method of treating DTDS comprising administering a therapeutically effective amount of a vector as described above to a patient with DTDS. Preferably, the patient is human.

When DTDS is “treated” in the above method, this means that one or more symptoms of disease are ameliorated. It does not mean that the symptoms are completely remedied so that they are no longer present in the patient, although in some methods, this may be the case. The method of treating results in one or more of the symptoms of the disease being less severe than before treatment. The method of treating may result in a plurality of the symptoms of the disease being less severe than before treatment.

A “therapeutically effective amount” refers to an amount effective, at dosages and for periods of time necessary, to achieve the desired therapeutic result, such as raising the level of functional protein in a subject (so as to lead to a level sufficient to ameliorate the symptoms of the disease).

The method of treatment causes an increase in the level of functional DAT protein in the subject. In some embodiments, the method of treatment causes an increase in the level of functional DAT protein to about a normal level (i.e. the level found in a normal healthy subject). In one embodiment, the method of treatment causes an increase in the level of functional DAT protein to, at most, normal levels.

The vector may be administered in any suitable way so as to allow expression of the SLC6A3 gene in the neurons. In particular embodiments, a single administration of the vector can be used to provide gene expression to ameliorate the pathologies associated with DTDS. The vector may be administered intracranially. Intracranial administration is the direct delivery of the vector to specific areas of the brain. The vector may be administered to dopaminergic neurons, for example, by stereotactic delivery. The vector may be administered to dopaminergic neurons of the substantia nigra by intraparenchymal administration. Intracranial administration does not include subretinal administration, e.g. subretinal injection.

Administration of an effective dose of the compositions may be by routes standard in the art including, but not limited to, systemic, local, direct injection, intravenous, cerebral, cerebrospinal, intrathecal, intracisternal, intraputaminal, intrahippocampal, intra-striatal (putamen and/or caudate), intracortical, or intra-cerebroventricular administration. In some cases, administration comprises intravenous, cerebral, cerebrospinal, intrathecal, intracisternal, intraputaminal, intrahippocampal, intra-striatal (putamen and/or caudate), midbrain or intra-cerebroventricular injection. Administration may be performed by intrathecal injection with or without Trendelenberg tilting.

Direct delivery to the CNS may involve targeting the intraventricular space, either unilaterally or bilaterally, specific neuronal regions or more general brain regions containing neuronal targets. Individual patient intraventricular space, brain region and/or neuronal target(s) selection and subsequent intraoperative delivery of AAV may be accomplished using a number of imaging techniques (MRI, CT, CT combined with MRI merging) and employing any number of software planning programs (e.g., Stealth System, Clearpoint Neuronavigation System, Brainlab, Neuroinspire etc). Intraventricular space or brain region targeting and delivery may involve use of standard stereotactic frames (Leksell, CRW) or using frameless approaches with or without intraoperative MRI. Actual delivery of the vector may be by injection through needle or cannulae with or without inner lumen lined with material to prevent adsorption of the vector (e.g. Smartflow cannulae, MRI Interventions cannulae). Delivery device interfaces with syringes and automated infusion or microinfusion pumps with preprogrammed infusion rates and volumes. The syringe/needle combination or just the needle may be interfaced directly with the stereotactic frame. Infusion may include constant flow rate or varying rates with convection enhanced delivery.

Typically, a physician will determine the actual dosage which will be most suitable for an individual subject and it will vary with the disease, age, weight and response of the particular patient. The appropriate dosage can be determined by one skilled in the art.

The vector may be administered at a single point in time. For example, a single injection may be given with no repeat administrations.

Combination therapies are also contemplated by the disclosure. Combinations of methods of the invention with standard medical treatments (e.g., corticosteroids or topical pressure reducing medications) are specifically contemplated, as are combinations with novel therapies. In some cases, a subject may be treated with a steroid to prevent or to reduce an immune response to administration of the vector described herein.

Further, the invention provides the vector described above for use in therapy, for example, in the treatment of DTDS.

In addition, the invention provides the use of the vector as described above in the manufacture of a medicament for treating DTDS. The vector may be administered intracranially. The vector may be administered at a single point in time with no repeated administrations.

In another aspect of the invention, there is provided a host cell comprising the vector as described above. The host may be any suitable host.

As used herein, the term “host” refers to organisms and/or cells which harbour a nucleic acid molecule or a vector of the invention, as well as organisms and/or cells that are suitable for use in expressing a recombinant gene or protein. It is not intended that the present invention be limited to any particular type of cell or organism. Indeed, it is contemplated that any suitable organism and/or cell will find use in the present invention as a host. A host cell may be in the form of a single cell, a population of similar or different cells, for example in the form of a culture (such as a liquid culture or a culture on a solid substrate), an organism or part thereof.

A host cell according to the invention may permit the expression of a nucleic acid molecule of the invention. Thus, the host cell may be, for example, a bacterial, a yeast, an insect or a mammalian cell.

In another aspect of the invention, there is provided a transgenic animal comprising cells comprising the vector as described above. Preferably the animal is a non-human mammal, especially a primate. Alternatively, the animal may be a rodent, especially a mouse; or may be canine, feline, ovine or porcine.

A skilled person will appreciate that all aspects of the invention, whether they relate to, for example, the vector, the use, the method of treatment or the host cell for example, are equally applicable to all other aspects of the invention. In particular, aspects of the vector may have been described in greater detail than in some of the other aspects of the invention, for example, relating to method of treatment. However, the skilled person will appreciate where more detailed information has been given for a particular aspect of the invention, this information is generally equally applicable to other aspects of the invention.

All patent and literature references cited in the present specification are hereby incorporated by reference in their entirety.

### BRIEF DESCRIPTION OF THE DRAWINGS

The invention will now be described in detail by way of example only with reference to the figures in which:

FIG. 1 shows loss of DAT function in DTDS can be restored both pharmacologically and using a gene therapy approach in the mDA model. (A) Uptake of tritiated dopamine at d65 after neurons treated for 24 h with pifithrin-μ (pif). Values are relative to protein concentration (n=3 each). (B) Measurement of tritiated dopamine uptake at d65 in patient-derived mDA neurons transduced with either a lentivirus construct expressing GFP alone (LV GFP) or human DAT and GFP (LV DAT-GFP) (n=3 for each). (C) Immunofluorescence analysis at d65 for patient-derived dopaminergic neurons transduced with LV GFP or LV DAT-GFP. Cells are stained for TH/MAP2, nuclei were counterstained with DAPI. Scale bar 100 μm. (D) Quantification of MAP2 positive, TH positive and TH/MAP2 double positive neurons at d65 of differentiation in mDA neurons transduced with LV GFP or LV DAT-GFP (n=3 for each). Both DTDS lines were independently compared to controls using two-tailed Student's t-test for all analyses.

FIG. 2 shows neonatal intracerebroventricular gene therapy to DAT knockout mice. (A) Weights of mice (2.25×10¹⁰ vg/pup, treated knockout n=13, wildtype n=12, untreated knockout=17 (Data means±S.E.M., Student's one-tailed t-test on weight at 365 days untreated knockout versus treated). (B) Kaplan-Meier survival plot of wildtype, untreated knockout, intracerebroventricular hDAT gene therapy treated knockout (Logrank, Mantel-Cox test). (C) Locomotor assessment of mice in open field with distance travelled. (D) Central time and thigmotaxis with representative open field traces for each group. (E) Vertical pole descent time. (F) Foot faults (Data means±S.E.M., two-way ANOVA, Log transformed data for % foot fault, Bonferroni's multiple comparison, group sizes as stated). (G) Representative immunostaining for mouse DAT in wildtype mice for physiological expression reference. Immunostaining for human DAT in treated knockout, untreated knockout and wildtype mice (scale bar 1 mm, n=5 per group). (H) Dopamine and serotonin neurotransmitter metabolites from whole brain homogenates analysed by HPLC. (Data means±S.E.M., two-way ANOVA, Bonferroni's multiple comparison, n=6 per group). (I) Representative immunofluorescence for cell types TH mDA neurons (scale bar 250 μm), striatal DARPP32 and GAD67 neurons (scale bar 100 μm) in wildtype, untreated knockout and knockout hDAT treated mice (n=5-6 per group). Data means±S.E.M. (J) Quantification of TH, DARP32 and GAD67 neurons in wildtype, untreated knockout and knockout hDAT treated mice (Data means±S.E.M. two-way ANOVA, Tukey's multiple comparison, n=5 per group). (K) Patch clamp electrophysiology of striatal medium spiny neurons (n=3 animals per group with 9-11 neurons measured per animal). Data means per animal ±S.E.M of group.

FIG. 3 shows adult stereotactic AAV2 gene therapy to DAT knockout mice 2 log dose-ranging study. (A) Weights of mice receiving stereotactic injected AAV2.hDAT vector treated knockouts at 3 dosages. 2×10¹⁰, 2×10⁹, 2×10⁸ vg/mouse. Control wildtype and knockout animals received AAV2.GFP vector 2×10¹⁰ vg/mouse (data means±S.E.M, n=8 per group). (B) Kaplan-Meier survival plot of wildtype AAV2.GFP, knockout AAV2.GFP and treated knockout AAV2.hDAT 2×10¹⁰, 2×10⁹, 2×10⁸ vg/mouse dosage groups. (C) Locomotor assessment of mice at 12 weeks (8 weeks post gene transfer) in open field with distance travelled. (D) Thigmotaxis central time. (E) Vertical pole descent time. (F) Foot faults (Data means±S.E.M., two-way ANOVA, Log transformed data for 25% foot fault, Bonferroni's multiple comparison n=5-8 animals per group). (G) Representative immunostaining of midbrain and striatum for human DAT in AAV2.hDAT treated knockout mice at 2×10¹⁰, 2×10⁹, 2×10⁸ vg/mouse dosages, (scale bar 100 μm, n=3 per group). (H) Representative double labelled immunofluorescence for TH mDA neurons coexpressing hDAT in AAV2.hDAT treated knockout mice at 2×10¹⁰, 2×10⁹, 2×10⁸ vg/mouse (scale bar 250 μm, n=3 per group). (I) Quantification of TH neurons of AAV2.hDAT treated knockouts at 2×10¹⁰, 2×10⁹, 2×10⁸ vg/mouse (Data means±S.E.M., two-way ANOVA, n=3 group). J Neurohistological panel showing frontal cortex of wildtype AAV2.GFP, knockout AAV2.GFP and knockout AAV2.hDAT treated mice at 2×10¹⁰, 2×10⁹, 2×10⁸ vg/mouse. Representative images of Haematoxylin and Eosin and Nissl stain (scale bar 250 μm). Immunohistochemistry for GFAP in in frontal cortex (scale bar 100 μm, n=3 per group for each panel).

FIG. 4 shows in vivo AAV9 hSyn GFP marker gene study (A) Illustration of AAV-hSyn-GFP construct used to for in vivo gene transfer to assess brain and mDA neuronal transduction. The expression cassette contains the hSyn 1 promoter driving GFP expression followed by a Woodchuck Hepatitis Virus Posttranscriptional Regulatory Element and polyadenylation signal (not drawn to scale). (B) GFP immunofluorescence of brain sections from prefrontal cortex to pons following intracerebroventricular delivery of AAV9-hSyn-GFP vector to neonatal wildtype mice and brain tissue collected at P35. Representative of 4 replicates. C Representative images of double labelled GFP and TH positive mDA neurons transduced with AAV-hSyn-GFP following neonatal Intracerebroventricular delivery. D Illustration of AAV-hSyn-SLC6A3 construct used for in vivo gene therapy experiments. The expression cassette contains the hSyn promoter driving hDAT expression followed by a Woodchuck Hepatitis Virus Posttranscriptional Regulatory Element and polyadenylation signal (not drawn to scale).

FIG. 5 shows electrophysiological properties of Medium Spiny Neurons following neonatal AAV9 hDAT gene therapy. (A) Current clamp recordings were performed on visually identified Medium Spiny Neurons in the dorsal striatum (Bregma 0.98-0.5; Scale bar=10 μm). (B) Representative traces of APs elicited by 300 pA current injection in wild-type (black traces), untreated knockout (red trace) and knockout hDAT (grey traces). wild-type and knockout hDAT showed two different populations of Medium Spiny Neurons while knockout only the one more excitable. (C) Top. Number of APs vs injected current for all the experimental data: wild-type (black n=24), untreated knockout (red n=22) and knockout hDAT (grey n=21). Middle. Percentage of high and low firing frequency in the 3 experimental groups. Bottom. Frequency distribution (%) of the firing rate for wild-type (black), untreated knockout (red) and knockout hDAT (grey). (D) Maximum firing rate, Current threshold and RMP showed as mean±SEM for wild-type, untreated knockout and knockout hDAT LD divided for high and low frequency firing rate.

FIG. 6 shows higher dosage in vivo neonatal AAV9.hDAT gene therapy. (A) Weights of mice. (B) Kaplan-Meier survival plot of wild-type, untreated knockout, and knockout mice treated with neonatal intracerebroventricular gene therapy with tenfold higher dosage of AAV9.hDAT gene therapy at P0 (2.25×1011 vector genomes) n=12: 7 males, females from 4 litters. (C) Locomotor assessment of mice at 3 monthly intervals with open field with distance travelled (D) thigmotaxis (E) vertical pole descent time (F) foot faults (2-way ANOVA, Log transformed data for % foot fault, Bonferroni's multiple comparison). (G) Immunostaining for mouse DAT in wild-type mice for physiological expression reference. Immunostaining for human DAT in higher dosage knockout, untreated knockout and wild-type mice. (scale bar 1 mm). (H) Representative image of double labelled immunofluorescence for % TH positive mDA co-expressing hDAT in knockout mice treated with AAV hDAT (scale bar=0.25 mm). (I) Quantification of co-expressing cells from knockout hDAT treated with high and low dosage group (n=6 per group, error bars represent s.e.m. and no significant difference (n.s.) on Student's t-test). (J) Dopamine and serotonin neurotransmitter metabolites from whole brain homogenates analysed by HPLC. (Numbers of animals stated, 2-way ANOVA, Bonferroni's multiple comparison). (K) Neuronal counts of TH, DARP32 and GAD67 positive neuronal subtypes from midbrain and striatal sections in wild-type, untreated knockout and high dose treated knockout at 356 days (n=6 animals per group, Two way ANOVA, Tukey's multiple comparison). (L) Representative images of immunofluorescence of mDA TH neurons (scale bar=0.25 mm) and striatal DARP32 and GAD67 neurons (scale bar=0.1 mm) from wild-type, untreated knockout and high dose treated knockout (n=6 per group). (M) Histological of brain cortex of wild-type, untreated knockout and knockout hDAT treated mice at lower dosage at 1 year. Knockout mice treated with 10 fold higher dosage showed 50% survival and were analysed at 1 year and those with reduced survival at P35. Haemotoxylin and Eosin and Nissl stain in brain cortex (scale bar 0.25 mm). Immunohistochemistry for GFAP and CD68 (scale bar 0.1 mm). Representative images are shown for 5 animals per group.

FIG. 7 shows In vitro AAV2.hDAT gene therapy (A) Schematic representation AAV2 vectors generated towards clinical application. (B) immunocytofluorescence of GFP and hDAT of knockout primary neurons transduced with AAV2.GFP or AAV2.hDAT vector (scale bar 0.1 mm). (C) Dopamine metabolite HPLC analysis of neuronal cell lysates of knockout primary neurons transduced with AAV2.GFP or hDAT. (D) Immunoblot of GFP and loading control mGAPDH E. Immunoblot for hDAT and loading control mGAPDH.

FIG. 8 shows in vivo stereotactic AAV2.hDAT gene therapy. (A) Schematic with projected experimental timeline and stereotactic target. (B) Open field trajectory traces of all animals tested at 12 weeks old (8 week post injection). (C) Immunoblot of MAO-A from midbrain slice with loading control mGAPDH. D Immunoblot of MAO-B from midbrain slice with loading control mGADPH. (D) Vector genomic copies of AAV2 vectors in injected brains. (E) Quantification of hDAT expression by qPCR in treated knockout mouse brains.

FIG. 9 shows neonatal intracerebroventricular gene therapy to DAT knockout mice using the enhanced Synapsin promoter. (A) Schematic of AAV hDAT gene therapy construct under transcriptional control of enhanced Synapsin promoter. This utilises a hybrid promoter combining human synapsin with CMV enhancer element. (B) Neonatal DAT-KO mice were treated at P1 with intracerebroventricular delivered AAV9.eSyn.hDAT gene therapy (1.9e11 vg/pup) to 16 pups. 4/16 (25%) treated mice developed parkinsonism and weight loss between P14-28. 75% showed normal survival to 1 year. (C) The growth of treated mice increased compared to untreated mice. Locomotor behavioural improved on testing; (D) distance travelled in open field, (E) exercise wheel, (F) vertical pole and (G) foot fault analysis at 3 months old. (H) Whole brain free floating hDAT immunohistochemistry revealed punctate transduction pattern in cortex, hippocampus and thalamus (scale bar 100 microns). (I) Double labelled immunohistofluorescence showed treated DAT KO mDA TH positive neurons expressing hDAT (scale bar 15 microns).

FIG. 10 shows in-life AAV2.hDAT gene therapy to 12 months. Symptomatic DAT knockout animals were treated with stereotactic AAV2.hDAT gene therapy at 3 doses and followed to 12 months. DAT knockouts and wildtype littermates receiving AAV2.GFP served as controls. Behavioural testing showing dose response with animals receiving highest dosage gene therapy showing survival (A) and motor behaviour equivocal to wildtype animals with (C) open field distance travelled (D) central time (E) descent times on vertical pole (F) foot faults with sustained efficacy to 12 months old.

FIG. 11 shows AAV2.hDAT gene therapy mediated DAT functional restoration through amphetamine response. The dopamine transporter is an obligatory target of amphetamine and this stimulant has no effect on locomotor activity in DAT knockout mice. (B) Restoration of DAT function is demonstrated through restoration of significant amphetamine response in all animals treated with AAV2.hDAT (C,D,E) with wildtype animals (A) serving as controls.

FIG. 12 shows immunohistochemistry showing safe dose-related targeted AAV2.hDAT expression to midbrain with no neurotoxic effects after 12 months. hDAT is expressed in DAT-KO midbrain in target dopaminergic and non-dopaminergic neurons (A). Neuropathology panel (B) Nissl, H&E, GFAP and CD68 at 12 months showing no neurotoxicity. Unexpectedly in the studies using Synapsin to drive hDAT expression, no neurotoxicity was observed in either the short or long term studies, as would be expected with off-target expression of hDAT.

### DETAILED DESCRIPTION OF THE INVENTION

Viral gene therapy uses recombinant viral vectors to deliver functional copies of gene to supplement genetic mutations to help restore gene, cell and organ function. Clinical examples of the impact of gene therapy have been reported in other childhood neurological disorders such as Spinal muscular atrophy type 1 and Aromatic L amino acid decarboxylase deficiency, a related paediatric neurotransmitter disorder.

The inventors have developed a clinically applicable gene therapy for DTDS that delivers a normal copy of the hDAT (SLC6A3) using an AAV2 viral vector. This gene therapy works by delivering a functional copy of the gene to DTDS patients to supplement the affected brain cells with a normal copy of the gene to restore DAT expression. The advantages of this approach are that it is applicable to all mutations that cause DTDS.

Using patient-derived induced pluripotent stem cells (iPSCs), the inventors generated a midbrain dopaminergic (mDA) neuron model of DTDS. Patient-derived neurons exhibited marked impairment of DAT activity, apoptotic neurodegeneration associated with TNFα-mediated inflammation and dopamine toxicity. Whilst DAT activity was ameliorated with the pharmacochaperone pifithrin-μ, the effect was mutation-specific. In contrast, lentiviral gene transfer restored DAT activity and prevented neurodegeneration in all patient-derived mDA lines.

The inventors undertook a proof-of principle study using DAT knockout mice as a model of DTDS. The model recapitulates human disease, exhibiting parkinsonism features, including tremor, bradykinesia and premature death. Neonatal intracerebroventricular injection of AAV9 vector provided neuronal expression of human DAT which ameliorated motor phenotype, lifespan and neuronal survival in the substantia nigra (SN) and striatum, though off-target neurotoxic effects were seen at higher dosage.

The inventors developed the AAV2 version of the hDAT gene therapy to restrict expression to target brain midbrain dopaminergic neurons. AAV2 serotype has been used in clinical trials of gene therapies delivered by intraparenchymal injection to the midbrain. The inventors injected young adult DAT knockout mice in a 2 log dose ranging scale study of AAV2.hDAT gene therapy delivered by stereotactic injection and identified dose response with efficacy in survival and behaviour. Importantly the modification with capsid and delivery method improved safety and the off-target neurotoxic effects were not observed.

Results

Loss of DAT Function and Dysregulated Dopamine Metabolism is Evident in DTDS Patient-Derived mDA Neurons

Using a patient-derived mDA model, the inventors first explored the effect of mutant DAT protein on neuronal function, comparing patient lines to age-matched and CRISPR-corrected controls.

iPSC lines were generated from dermal fibroblasts of DTDS patients with homozygous missense mutations in SLC6A3 (Patient 1: c.1103T>A, p.L368Q; Patient 2: c.1184C>T, p.P395L). Control iPSCs were similarly generated from an age-matched healthy individual. An isogenic control line was created by CRISPR-Cas9 correction of the c.1184C>T variant in Patient 2. Genomic DNA sequencing confirmed that all patient-derived iPSC lines maintained their specific homozygous SLC6A3 mutation, with successful correction of the mutation in the isogenic control. All iPSC lines exhibited pluripotency and maintenance of genomic integrity.

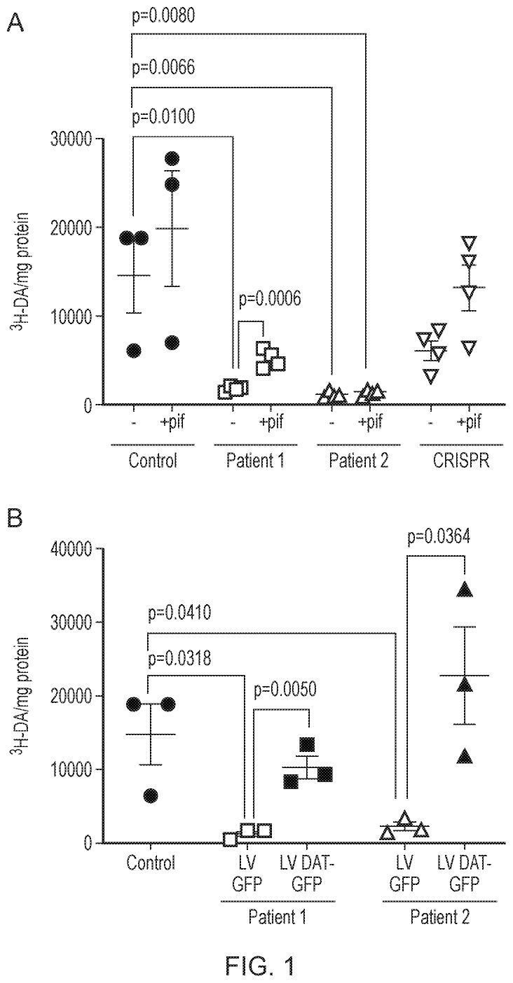

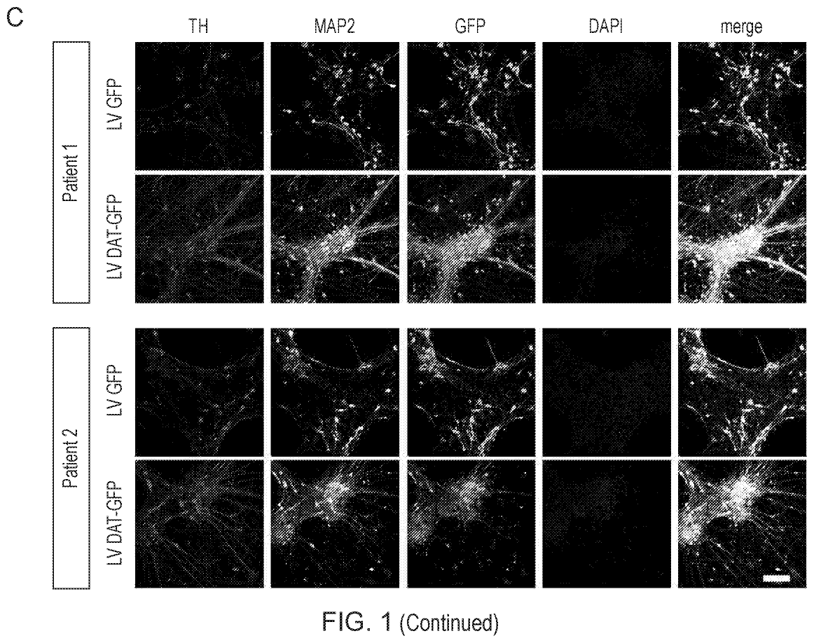

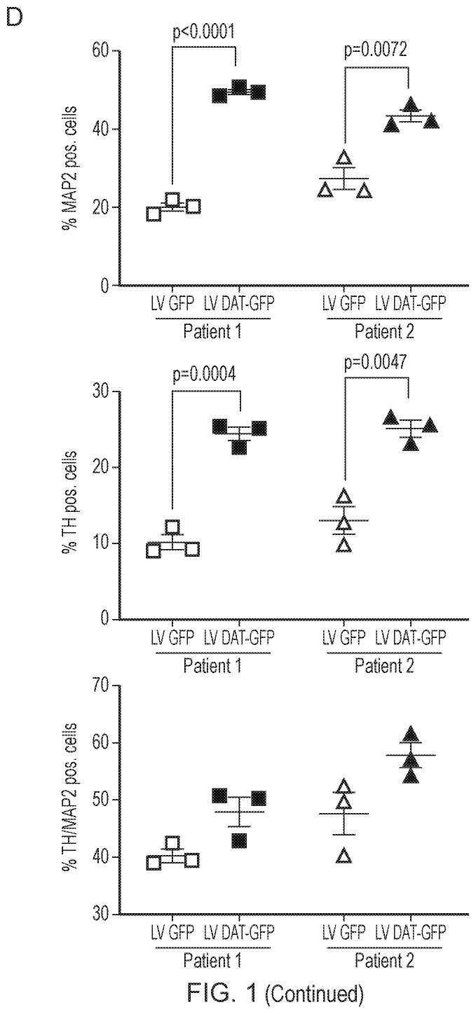

Having established disease-specific parameters in the DTDS iPSC—derived mDA model, the inventors utilised the model to validate targeted treatments for DTDS. Most missense variants in DTDS are associated with loss of transporter function, due to protein folding defects, retention in the endoplasmic reticulum (ER), and reduced surface expression of mature glycosylated transporter. Therefore, the inventors tested whether the Heat Shock Protein 70 (HSP70) inhibitor pifithrin-μ could rescue defective protein folding and restore DAT function in vitro. Mature mDA neurons at Day 65 were treated for 24 hours with pifithrin-μ, before measuring uptake of tritiated dopamine. Neurons derived from Patient 1 showed a significant two-fold increase in DAT activity, reaching 35% of mean dopamine uptake activity levels observed in control lines with no overall increase in total DAT protein (FIG. 1A). No increase in DAT activity with pifithrin-μ was observed for Patient 2 (FIG. 1A).

Given the mutation-specific effects of pifithrin-μ treatment, the inventors consequently sought to develop a gene therapy approach, applicable to a broader range of DTDS patients. A lentiviral construct was generated expressing human SLC6A3 gene under the transcriptional control of the neuron-specific promoter, human synapsin (hSyn1). Patient derived mDA precursors were transduced at Day 24 of differentiation and analysed at Day 65 of derived maturity. Lentiviral gene transfer led to restoration of dopamine uptake, to levels comparable to age-matched control lines (FIG. 1B). Despite this recovery of DAT activity, the inventors did not observe normalisation of dysregulated MAO-A and MAO-B enzyme levels by Day 65. Nonetheless, rescue of DAT function by gene therapy successfully halted neuronal loss, and more specifically, prevented dopaminergic neurodegeneration (FIGS. 1C,D).

Proof of Concept Gene Therapy of DAT Knockout Mice by Neonatal Intracerebroventricular Gene Transfer

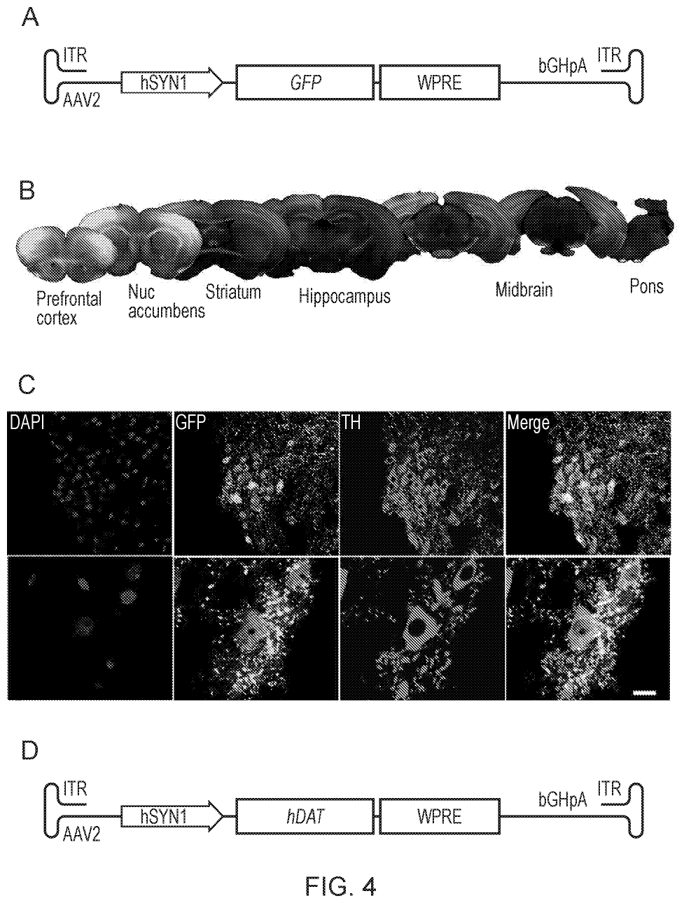

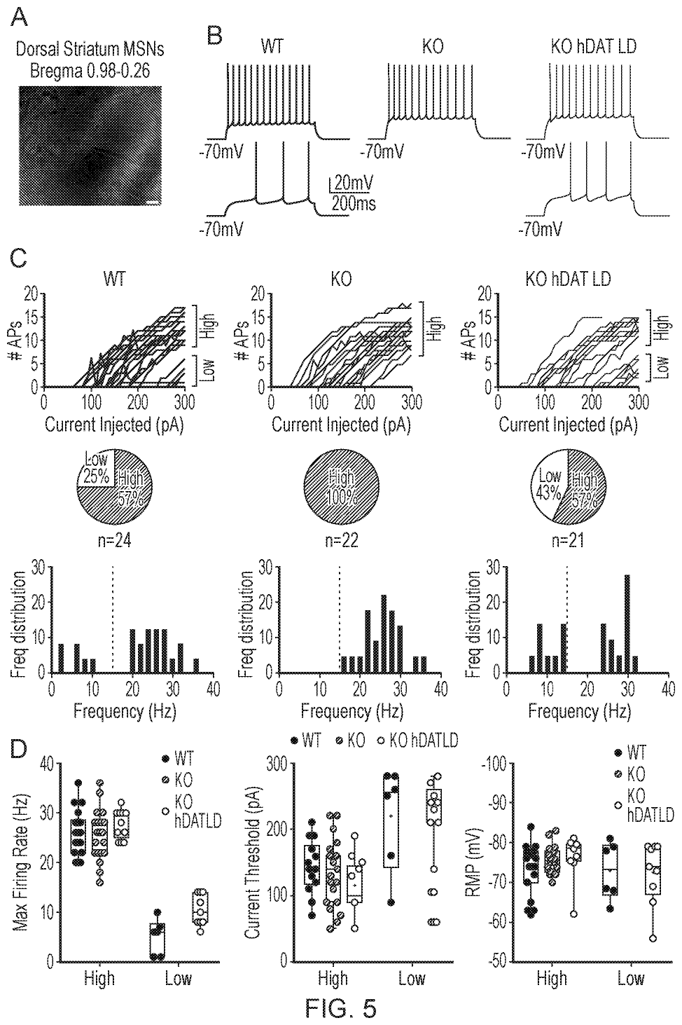

In preparation for in vivo preclinical gene therapy, the inventors injected adeno-associated virus serotype 9 (AAV9) vector encoding GFP under transcriptional control of a truncated hSyn1 promoter (2×10¹¹ vector genomes, n=4: 1 male, 3 females from a single litter) (FIG. 4A) into the lateral ventricle of neonatal wildtype mice. At 35 days, GFP expression extended bilaterally, from the prefrontal cortex to cerebellum and was, notably, present in mDA neurons (FIGS. 4B.C).

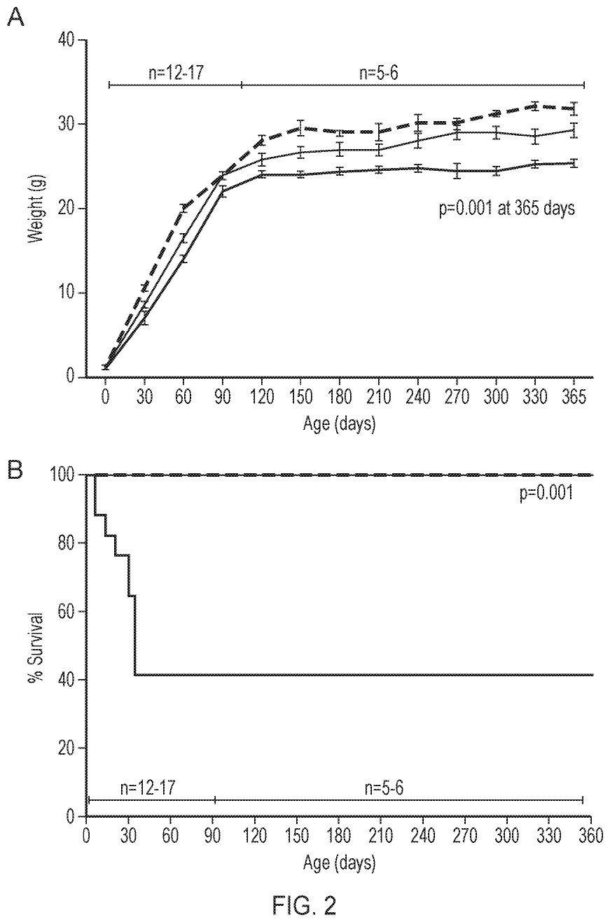

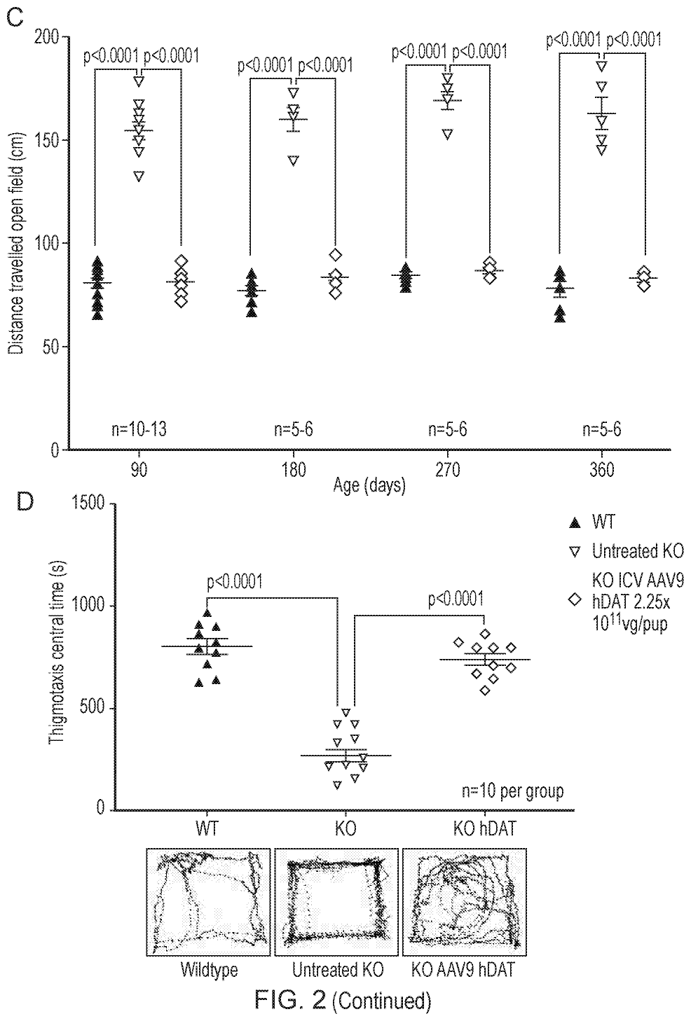

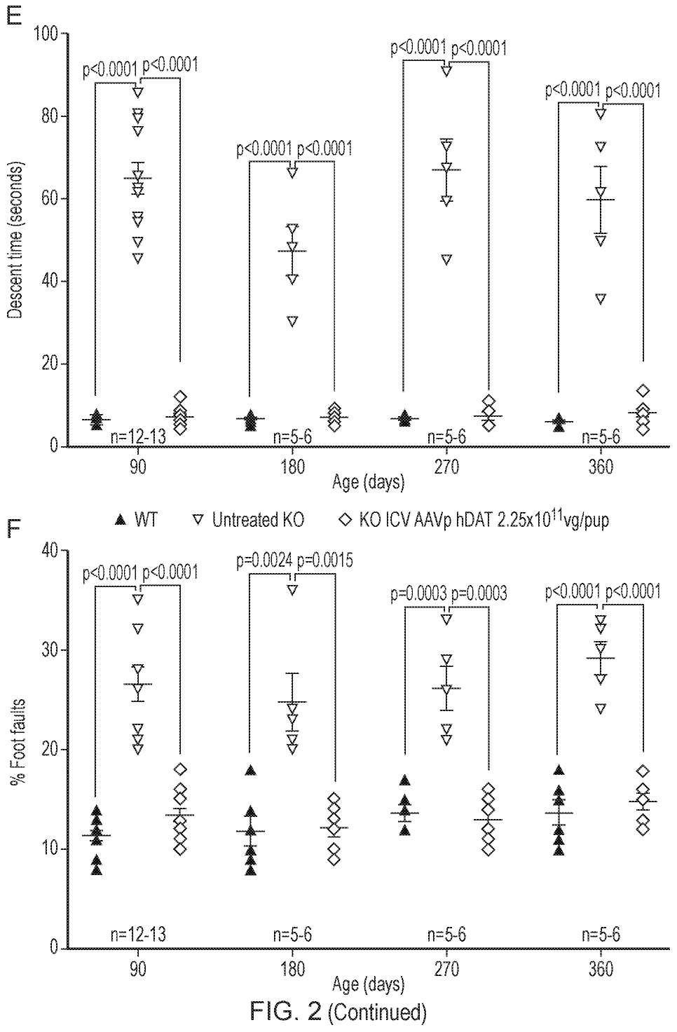

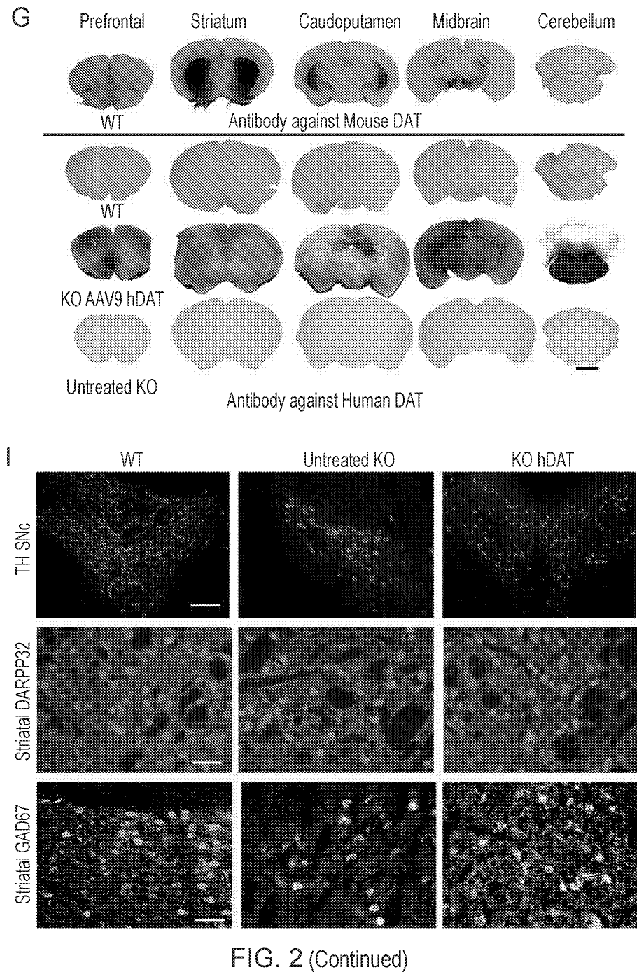

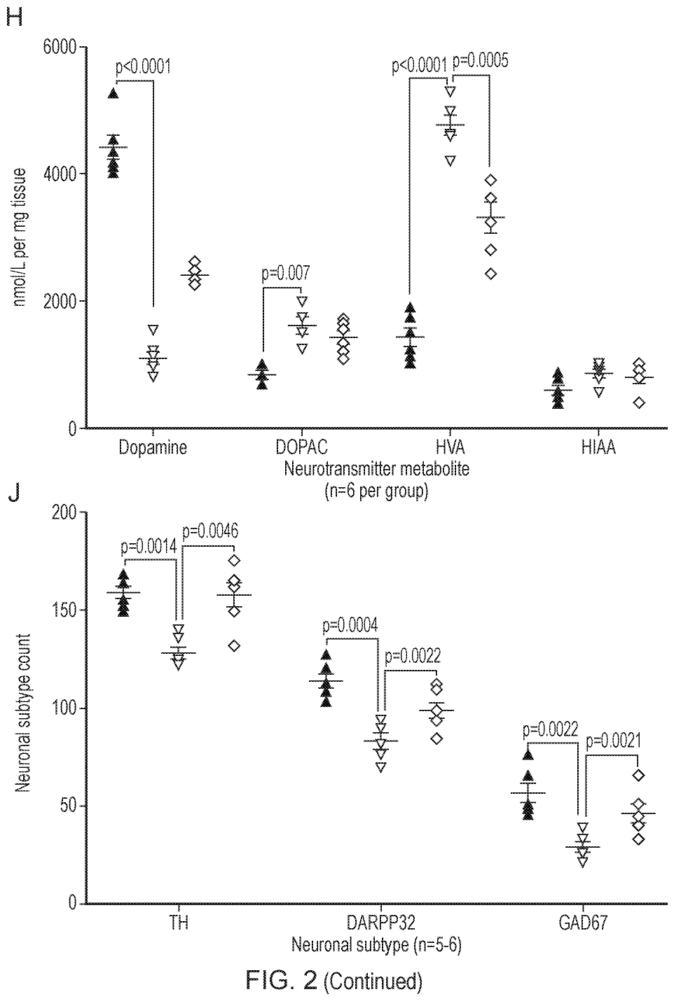

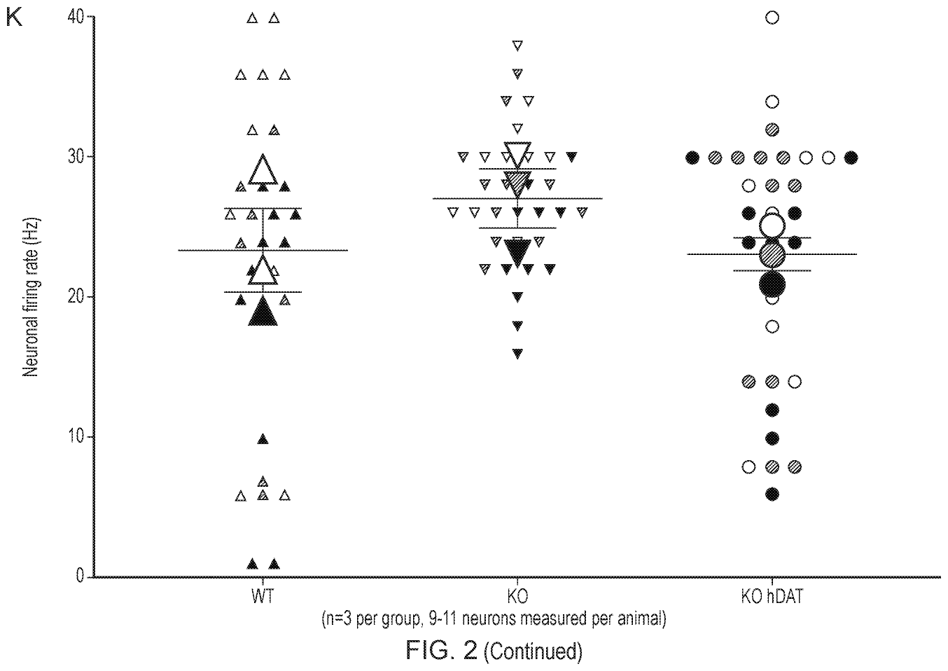

The inventors established baseline phenotype readouts in a previously-characterised DAT knockout mouse model (B. Giros et al., Nature 379, 606-612 (1996); M. Cyr et al., Proceedings of the National Academy of Sciences of the United States of America 100, 11035-11040 (2003)). Consistent with previous studies, all knockouts exhibited poor weight gain (FIG. 2A), displaying hyperlocomotor activity by P21 (data not shown) with 59% developing tremor, bradykinesia and weight loss (data not shown), reaching humane endpoint by P35 (n=10, 4 males, 6 females) (FIG. 2B).

The inventors generated an AAV9 vector for human DAT expression under transcriptional control of a truncated hSyn1 promoter (FIG. 4D). At P0, DAT knockout pups received intracerebroventricular injection of vector (2.25×10¹⁰ vector genomes (vg) per pup, n=13: 7 males, 6 females from 4 litters). Uninjected wildtype littermates (n=12: 5 males, 7 females from 5 litters) and knockout mice (n=17: 9 males, 8 females from 7 litters) served as controls. Treated knockouts were significantly heavier than surviving untreated knockouts (FIG. 2A). 10 out of 17 untreated knockouts required euthanization before 35 days; the remainder survived until tissue collection at 365 days. All treated knockouts and wildtype mice survived to the collection timepoints (FIG. 2B). Untreated knockouts were hyperactive, travelling significantly further distances and less central zone time in open field tests; treated knockouts were indistinguishable from wild type littermates in both distance travelled and central time (FIGS. 2C,D). Knockouts had a significantly prolonged descent time on the vertical pole test, and made significantly more foot faults; the performance of treated knockouts was indistinguishable from wildtype mice (FIGS. 2E,F). Treated knockouts did not develop parkinsonism (data not shown).

Treated knockouts expressed hDAT bilaterally from the prefrontal cortex to cerebellum including striatum and midbrain, where DAT is physiologically expressed (FIG. 2G). Whole brain homogenate from untreated knockouts had significantly reduced dopamine levels with raised DOPAC and HVA concentrations compared with wildtype mice; these differences were reversed, but not normalized, in treated knockouts (FIG. 2H). Gene therapy significantly ameliorated both dopaminergic and striatal neurodegeneration (FIGS. 2I,J). Patch clamp electrophysiology of medium spiny neurons in the dorsal striatum revealed the presence of two different populations in wildtype mice, exhibiting high and low firing rates. Only high-firing rate neurons were detected in the untreated knockouts. AAV.hDAT treatment of knockouts restored the bimodal firing distribution (FIG. 2K).

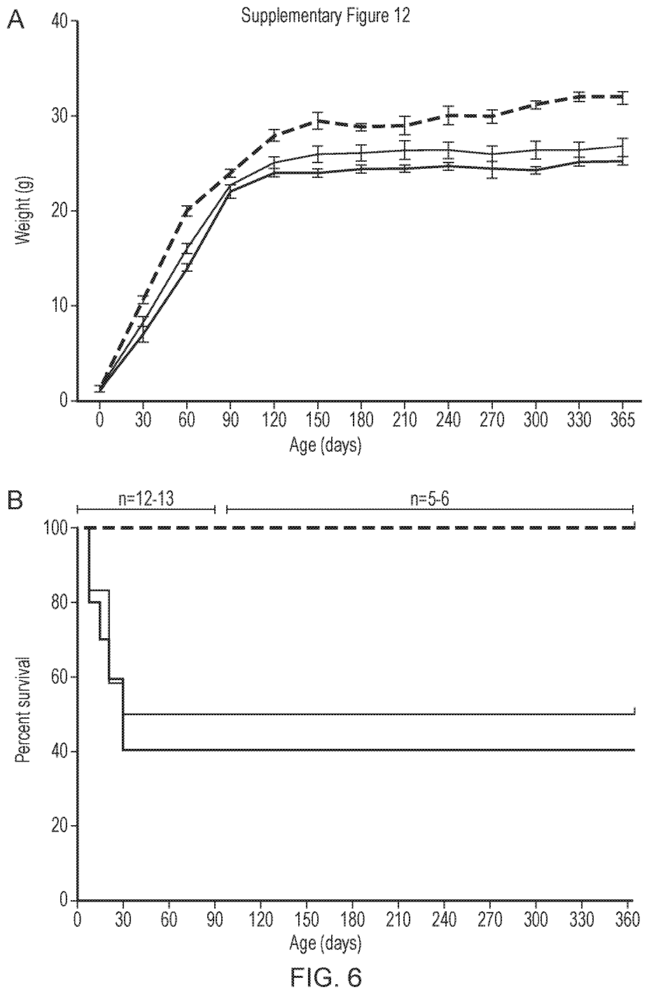

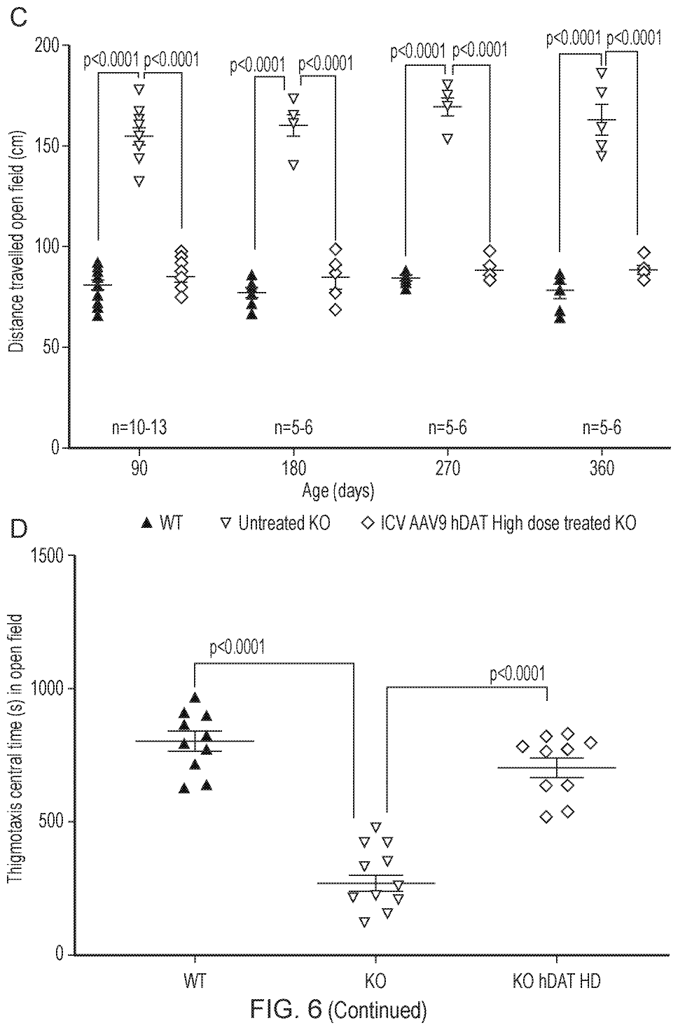

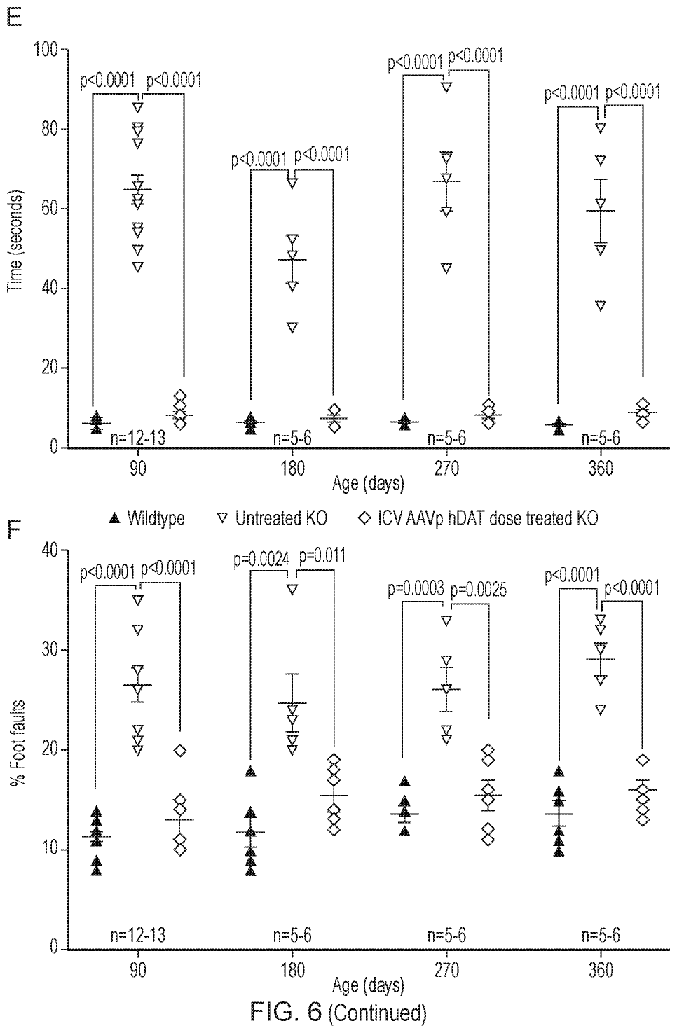

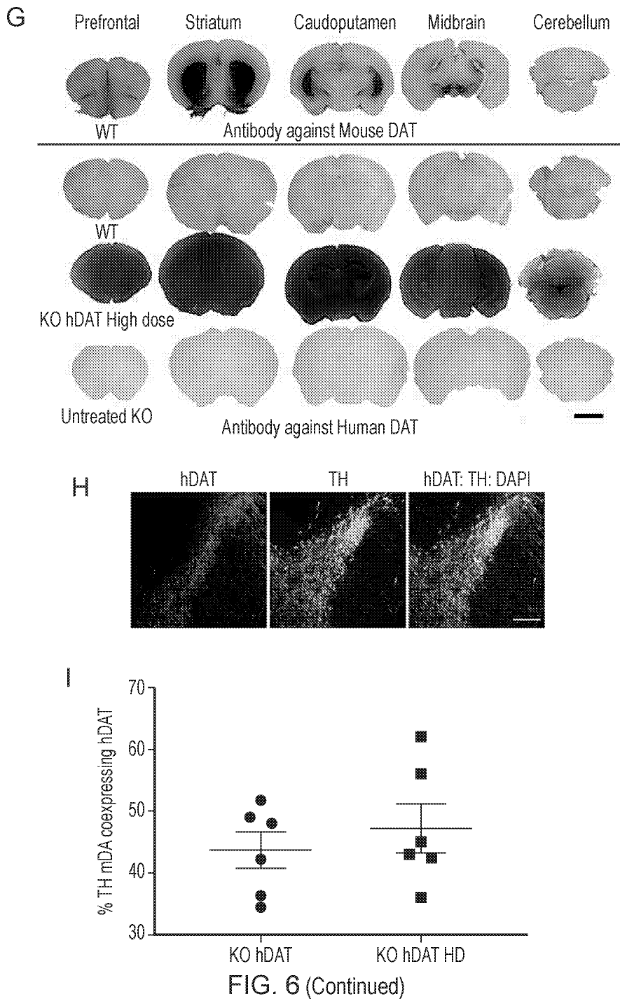

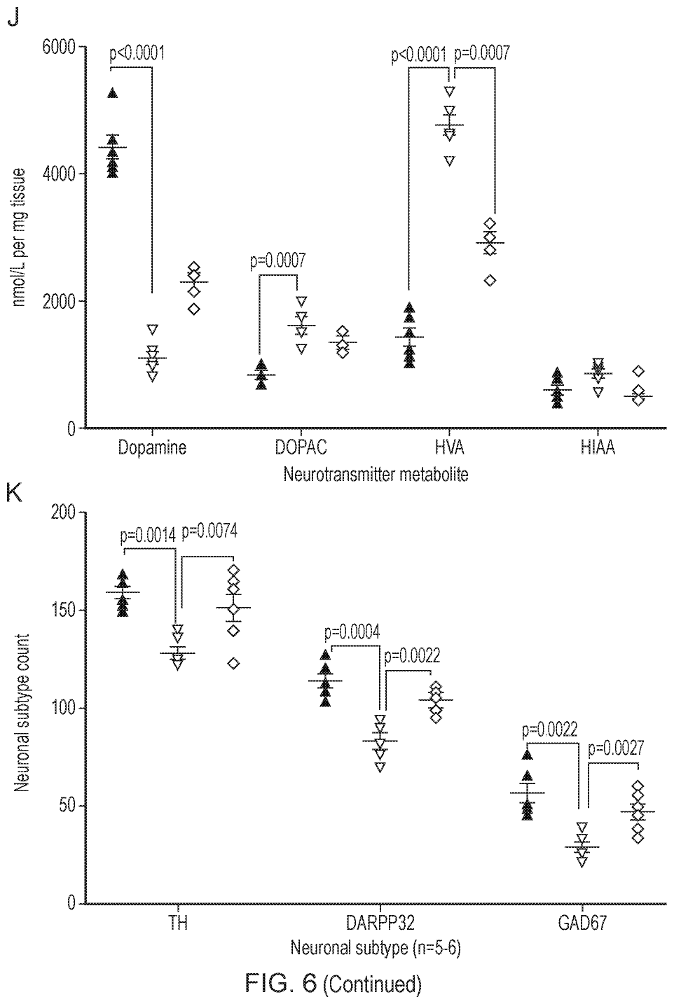

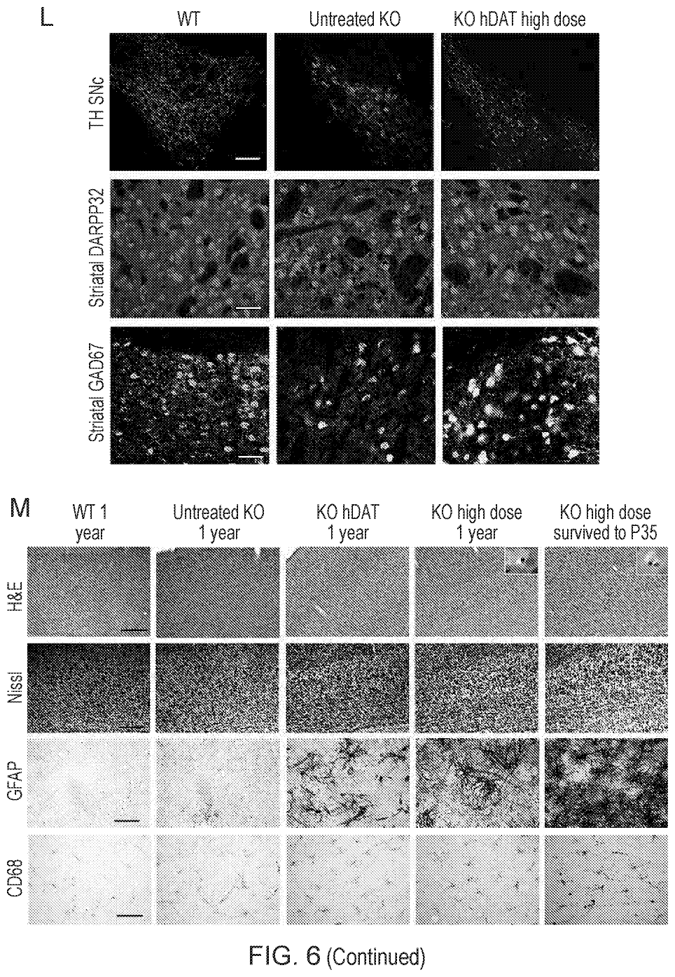

To attempt to fully restore dopamine homeostasis and neurotransmitter profile, a second knockout group received a tenfold higher dosage of intracerebroventricular AAV9.hDAT gene therapy at P0 (2.25×10¹¹ vg per pup, n=12: 7 males, 5 females from 4 litters) by injection. Treated mice were heavier than untreated knockouts, however 50% of them developed unexpected, early tremor, bradykinesia and weight loss necessitating euthanasia by P35 (FIGS. 6A,B). The remainder were indistinguishable, on motor behavioural testing, from wildtype animals and survived to sacrifice at 365 days (FIG. 6C-F). Bilateral hDAT expression was observed throughout the brain; however, despite receiving a ten-fold higher vector dose, mDA transduction was not significantly higher than in the lower dose cohort (FIG. 6G-I). Furthermore, despite restoration of HVA levels and correction of neurodegeneration (FIG. 6J-L), there was cortical cell loss and vacuolation with greatly increased GFAP expression in the cerebral cortex (FIG. 6M).

Preclinical Gene Therapy for DTDS— Targeted Delivery to the Substantia Nigra for Future Clinical Translation

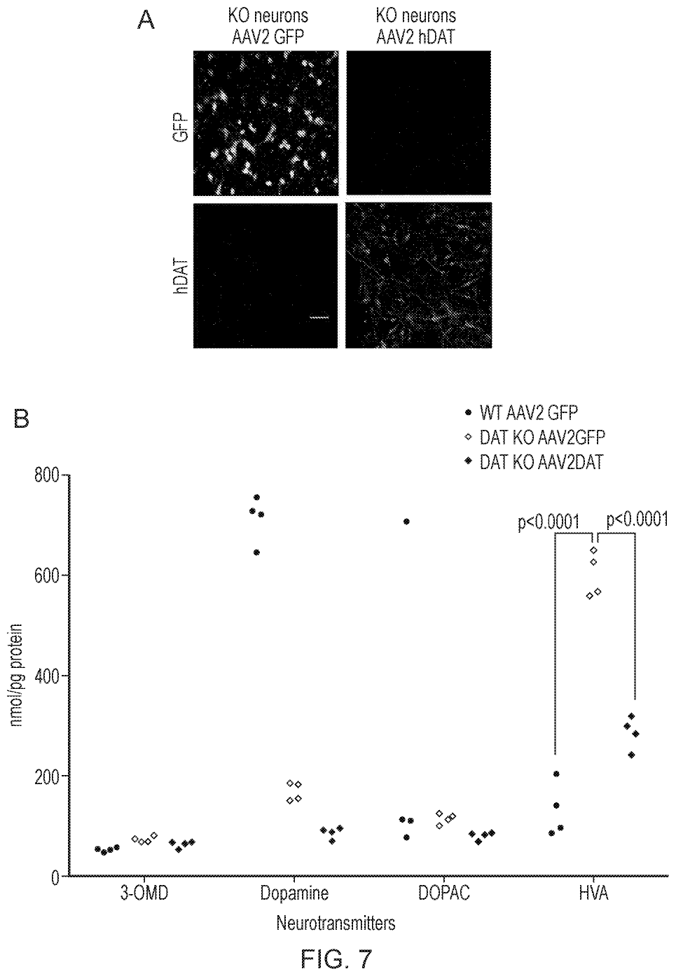

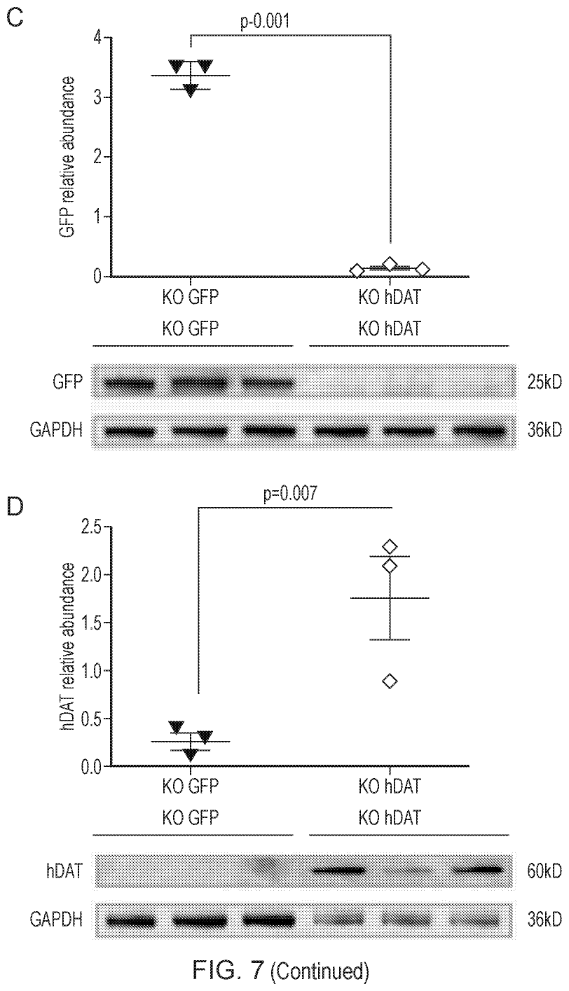

In order to move even closer towards clinical translation, the inventors further developed vector delivery to model clinical application and restrict expression to dopaminergic neurons by intraparenchymal stereotactic delivery. The inventors also chose to utilise AAV2 capsid as it exhibits restricted spread after CNS delivery. Primary DAT knockout neurons treated with AAV2.SLC6A3 vector expressed hDAT protein and exhibited dopamine uptake as indicated by reduction of HVA concentration (FIG. 7A-D).

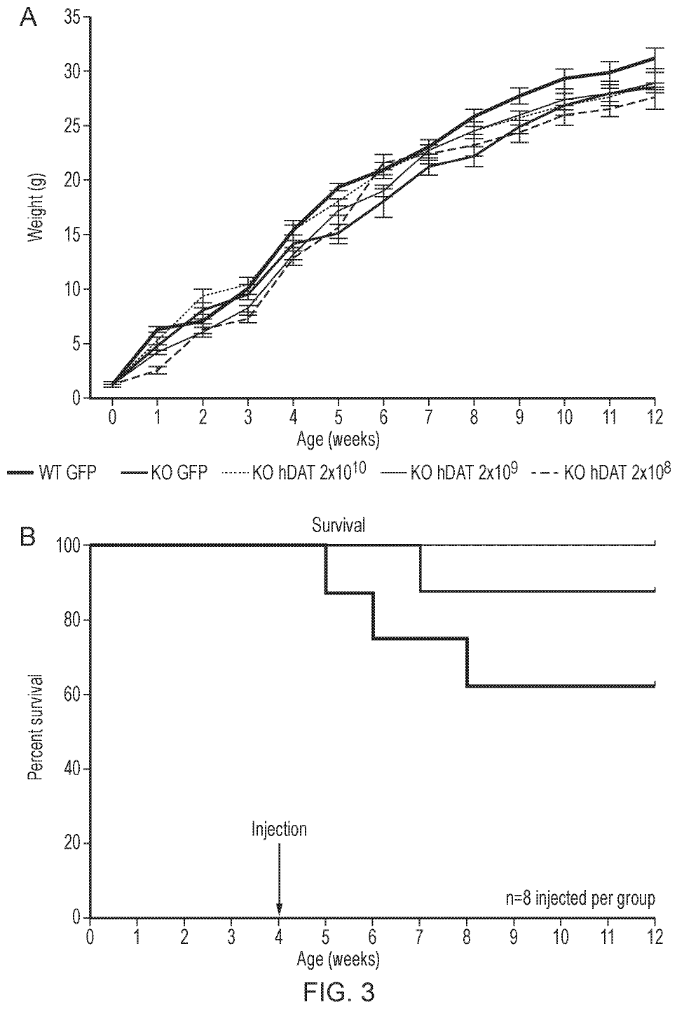

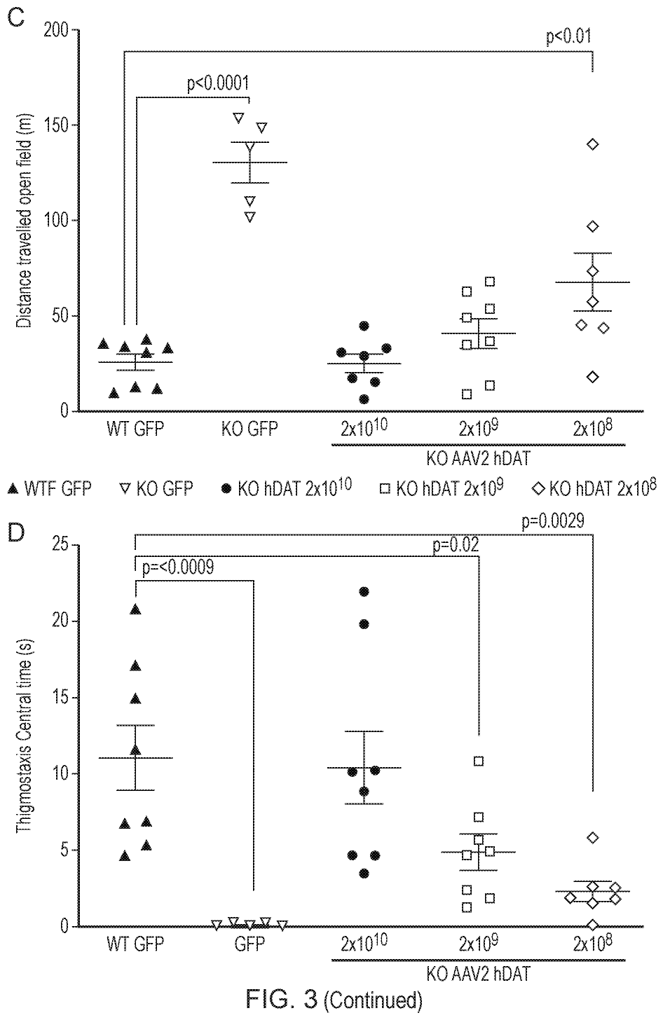

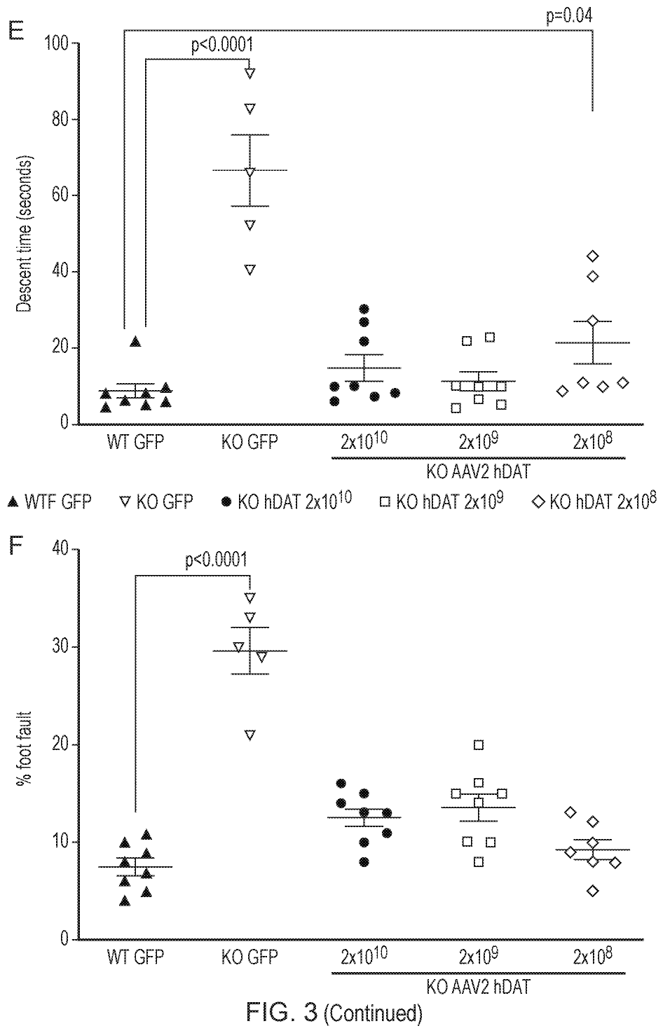

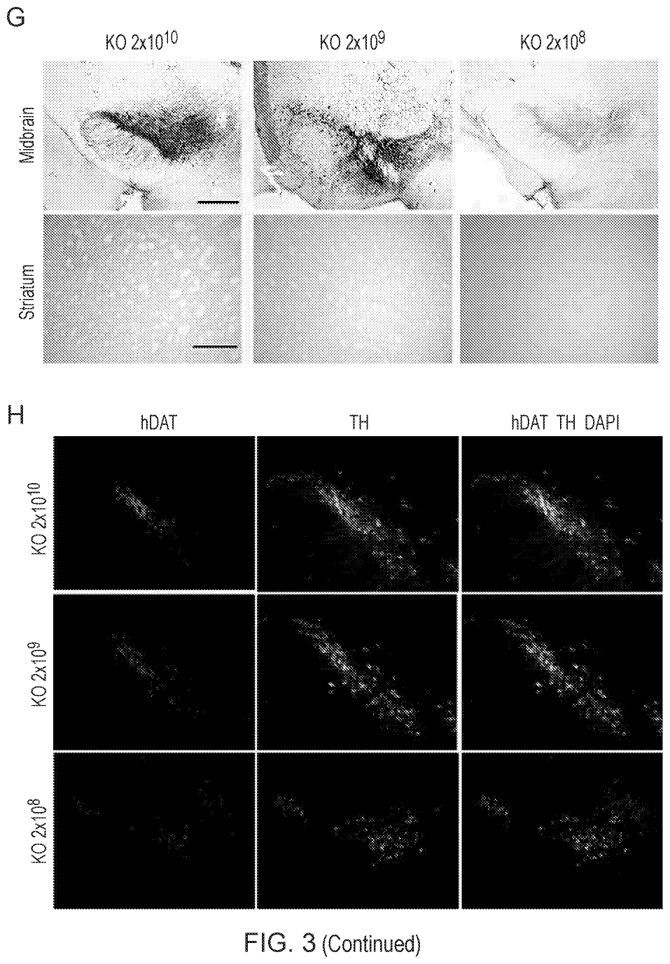

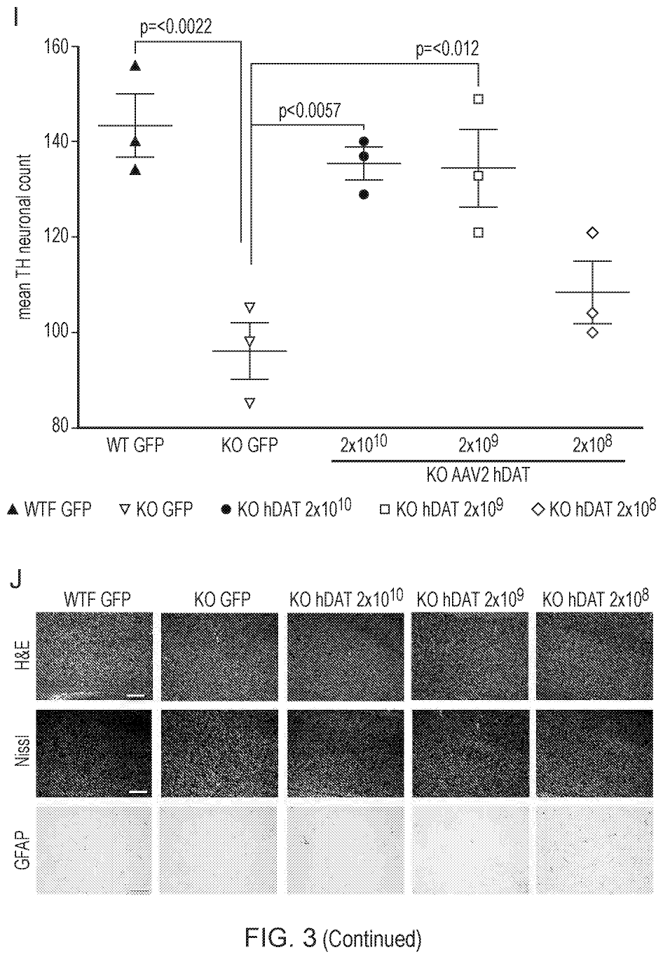

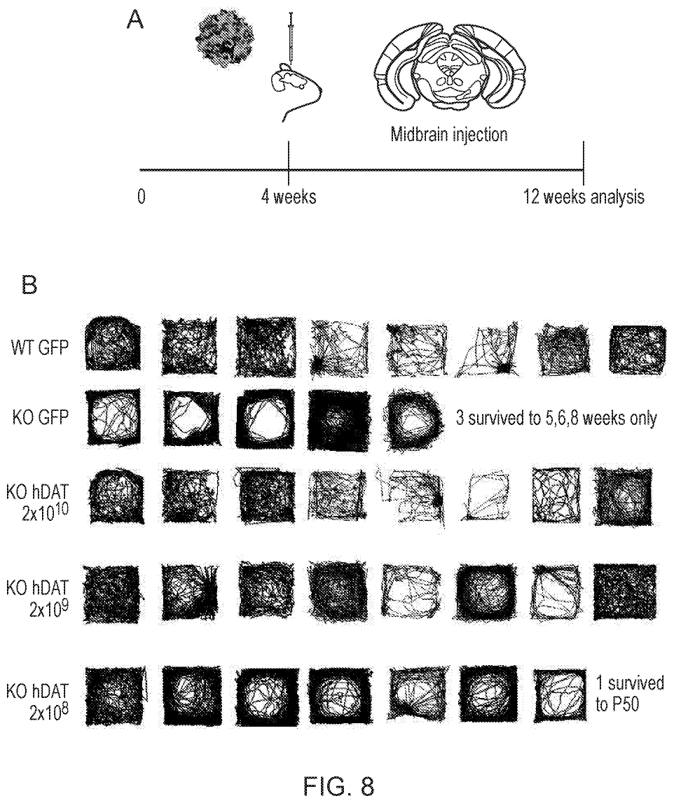

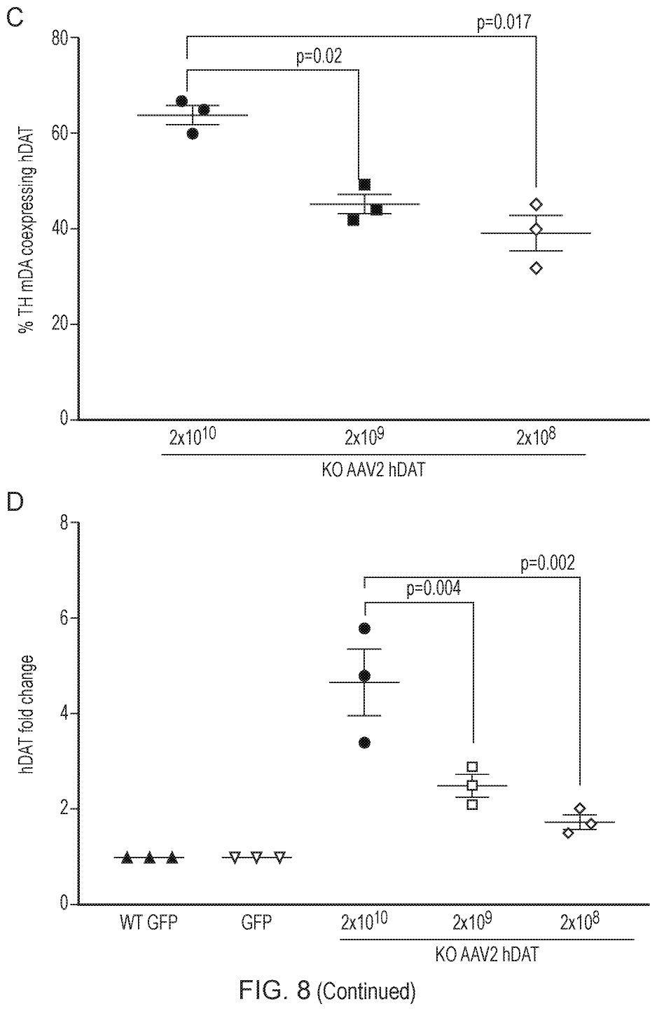

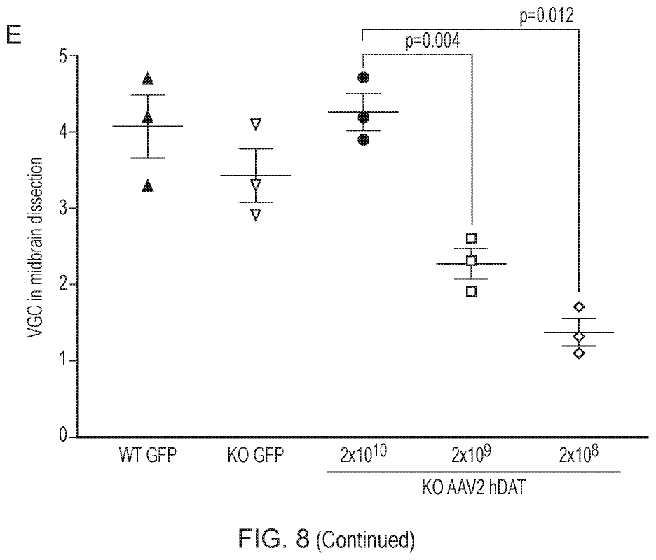

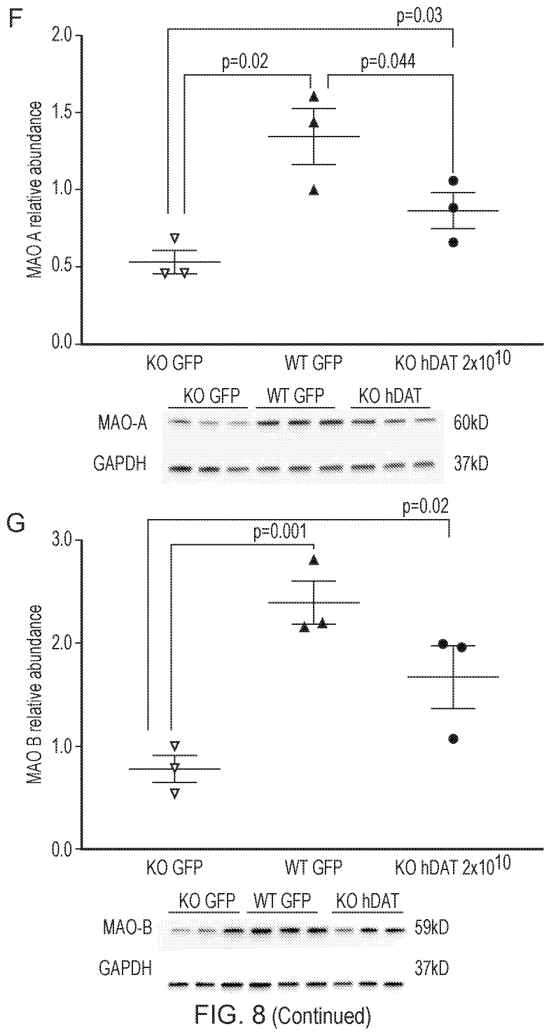

AAV2.SLC6A3 was delivered by targeted bilateral stereotactic injection to the substantia nigra (SN) of 4 week old symptomatic knockouts (modelling adolescent DTDS patients) at 3 dosages: neat=2×10¹⁰, 1;10=2×10⁹, 1;100=2×10⁸ vg/mouse respectively, n=8 per group, 13 males, 11 females from 6 litters). AAV2.GFP control vector was injected to wildtype and knockout littermate controls (2×10¹⁰ vg/mouse, n=8 per group, 7 males, 9 females from 4 litters) (FIG. 8A). Weights were not significantly different between knockout AAV2.GFP and AAV2.hDAT treated groups, irrespective of dosage (FIG. 3A). Survival was improved in all AAV2.hDAT treated animals compared to AAV2.GFP treated knockouts with 100% survival of the neat dosage group at 12 weeks of age (FIG. 3B). With the lowest dosage (2×10⁸ vg/mouse), one mouse developed weight loss and parkinsonism, surviving to P50. Three out of eight (37.5%) AAV2.GFP treated knockouts reached humane endpoint at 5, 6, 8 weeks. At 8 weeks post gene transfer behavioural testing (n=5-8 per group) showed knockout mice treated with highest dosage (2×10¹⁰ vg/mouse) displayed motor behaviour that was indistinguishable to AAV2.GFP treated wildtypes. (FIG. 3C-F, FIG. 8B). Dose response was observed in open field distance travelled and central time (FIGS. 3C,D, FIG. 8B). Vertical pole descent time was restored to wildtype levels in 2×10¹⁰ and 2×10⁹ vg/mouse dosages but not lowest dosage (2×10⁸ vg/mouse) whilst % foot faults were restored to wildtype levels in all treated knockouts (FIGS. 3E,F). hDAT staining in midbrain and striatum confirmed restricted expression to the midbrain with dose-dependent anterograde transport to the striatum (FIG. 3G).

Quantification of TH-positive mDA neurons expressing hDAT showed rescue of neurodegeneration (FIGS. 3H,I) which correlated with midbrain TH transduction, hDAT mRNA transcripts and vector genome copies (vgc) delivered (FIG. 3H, FIGS. 8C-E). In keeping with the iPSC-derived mDA model, knockout mice had significantly lower levels of MAO-A and MAO-B in the midbrain when compared to wildtype animals (P=0.02 and 0.001 respectively) (FIGS. 8F,G). Treatment with AAV2.SLC6A3 neat dosage significantly increased, but did not normalize these enzymes though (MAO-A P=0.03 and MAO-B P=0.02) (FIGS. 8F,G), as illustrated, lack of complete MAO-A/B restoration did not affect rescue of the murine model with regard to motor phenotype, weight, survival and neurodegeneration. In contrast to the intracerebroventricular approach, no cortical cell loss or vacuolation was observed with targeted stereotactic SN delivery (FIG. 3J).

AAV9.eSyn.SL6CA3.WPRE

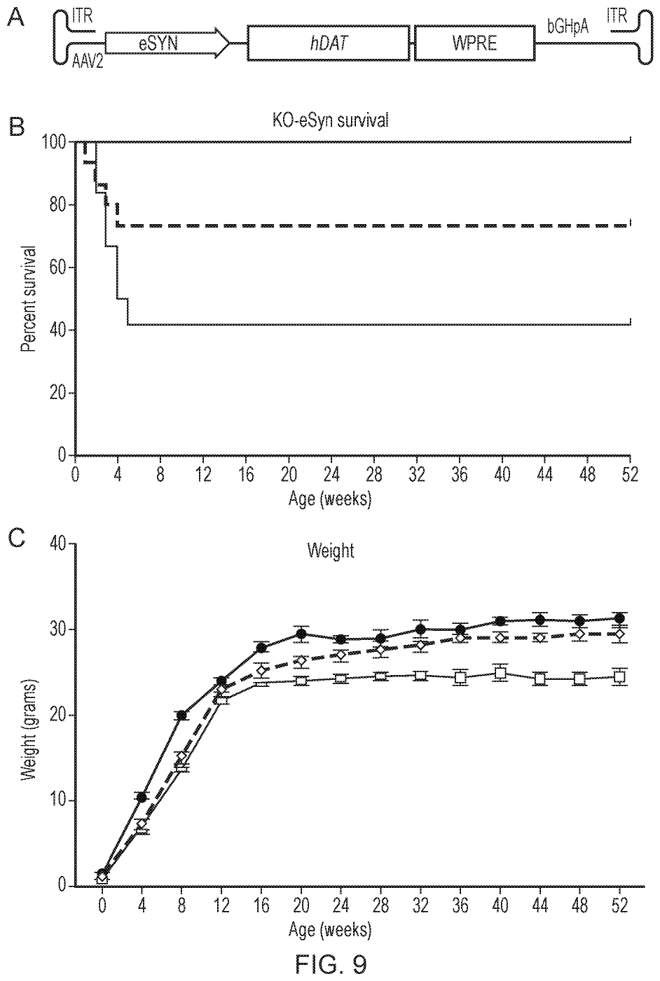

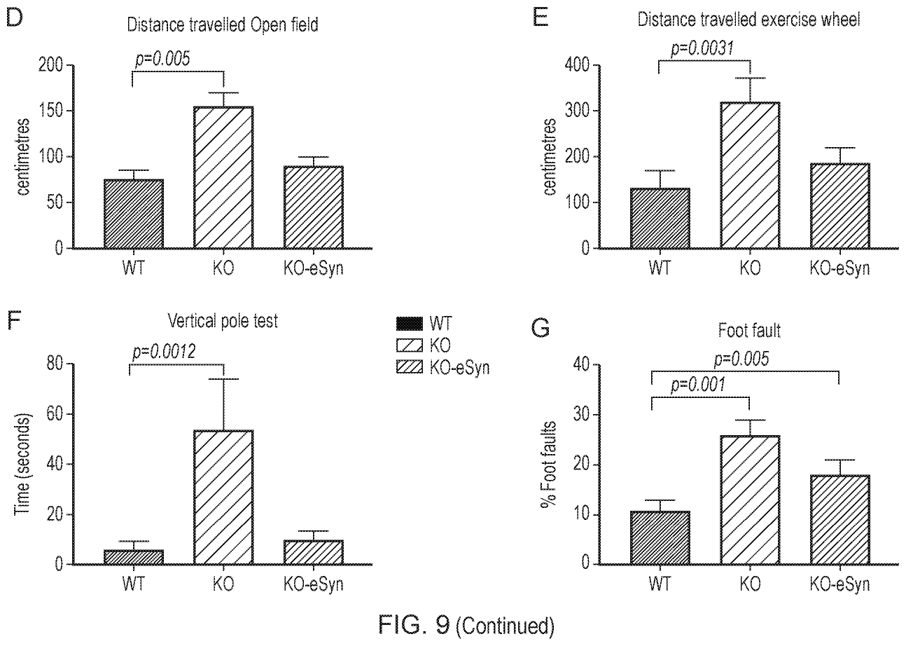

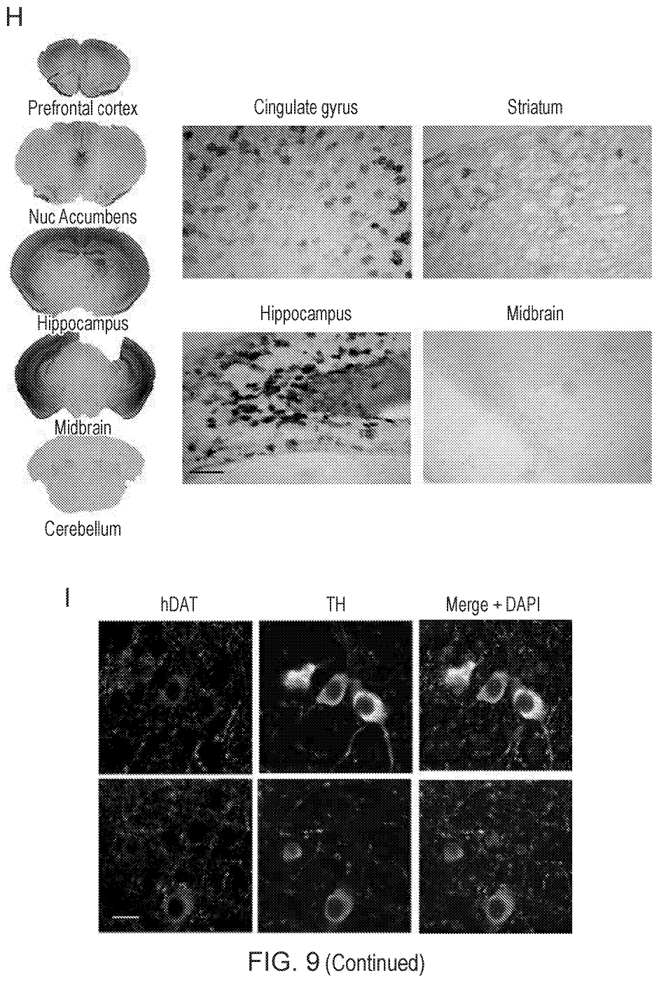

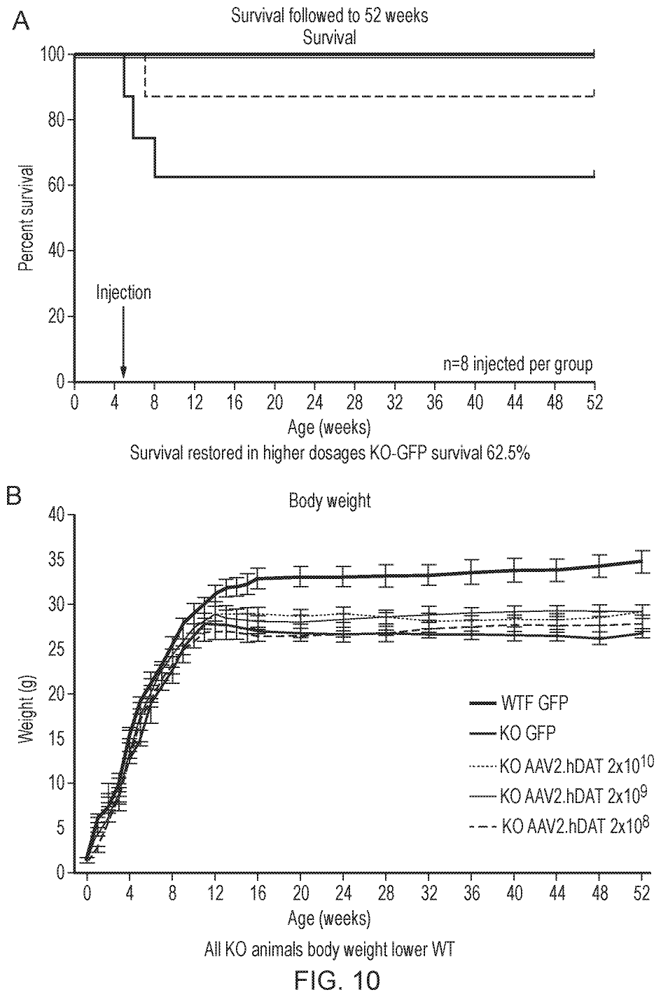

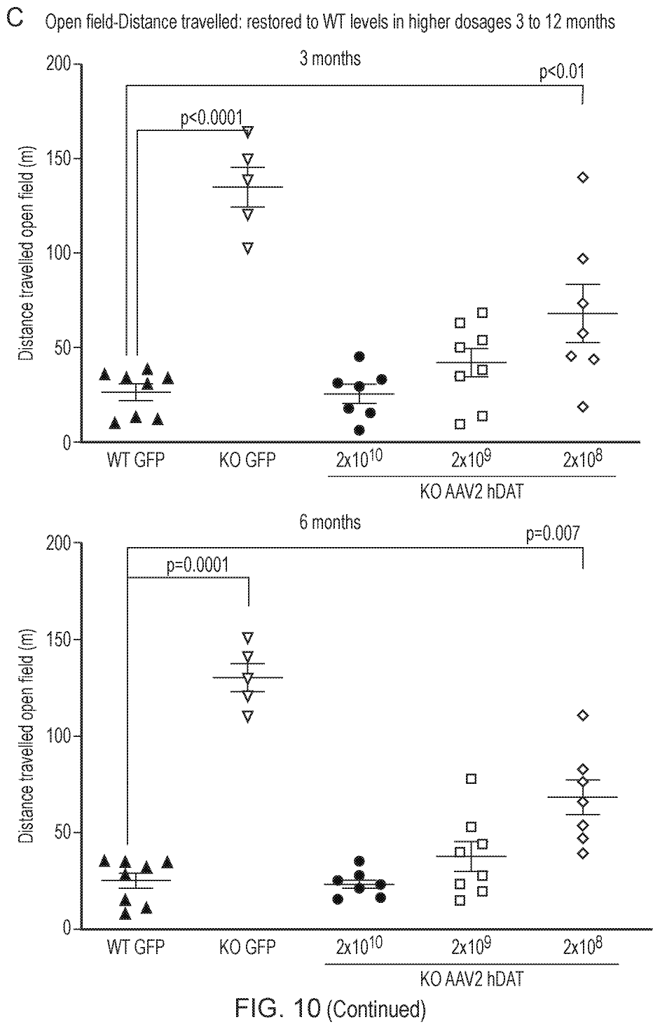

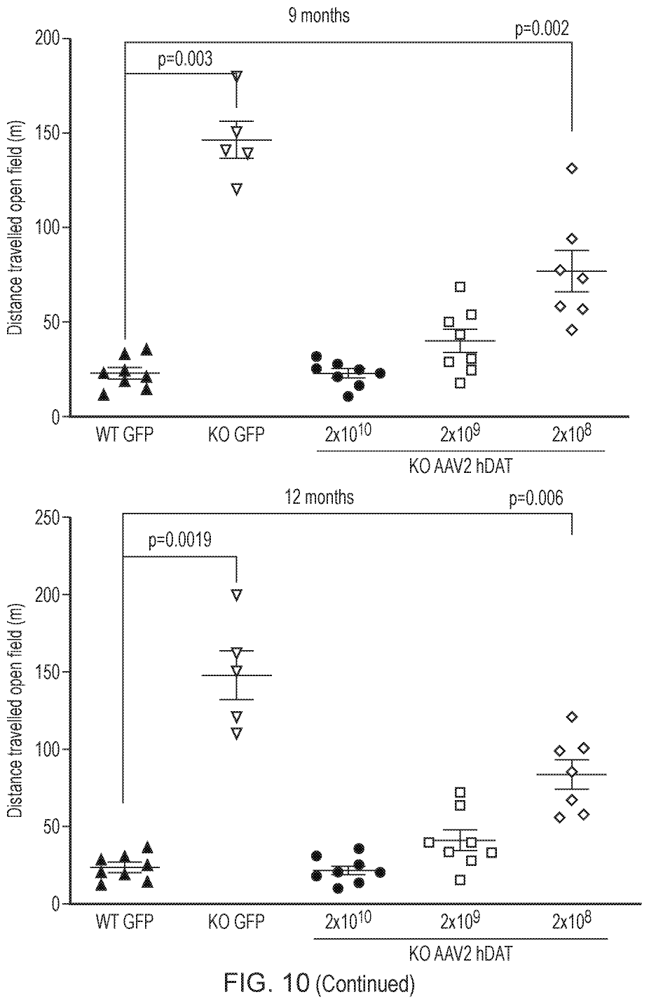

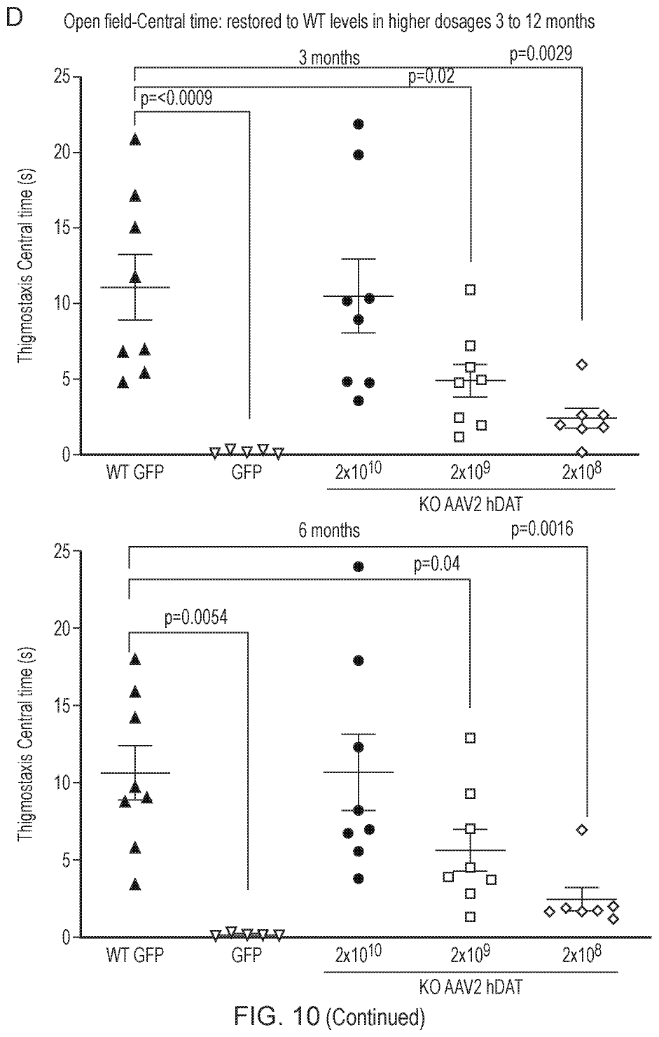

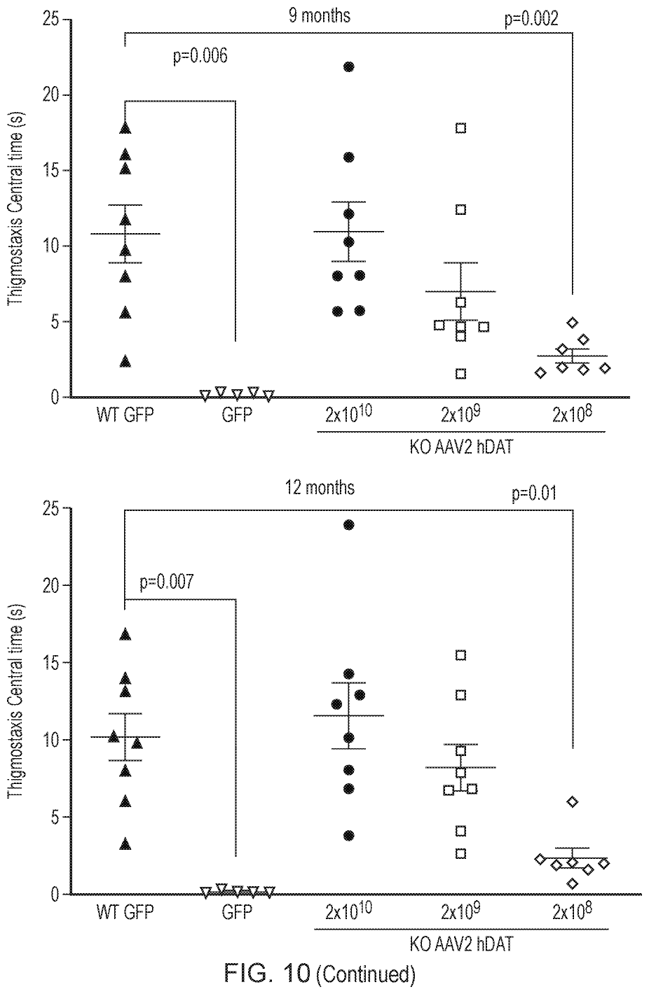

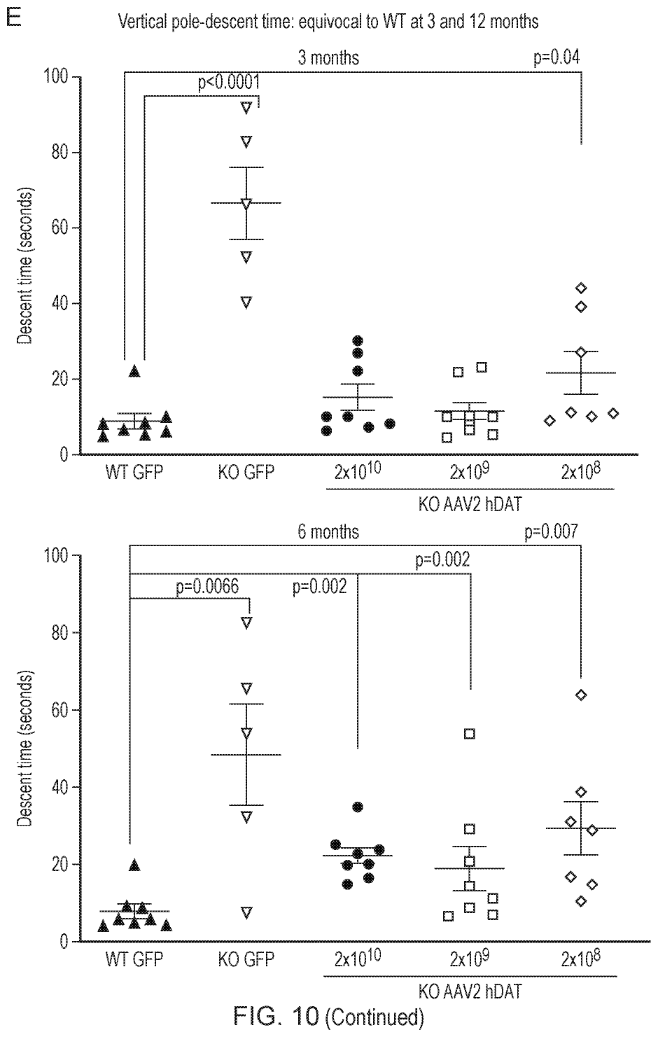

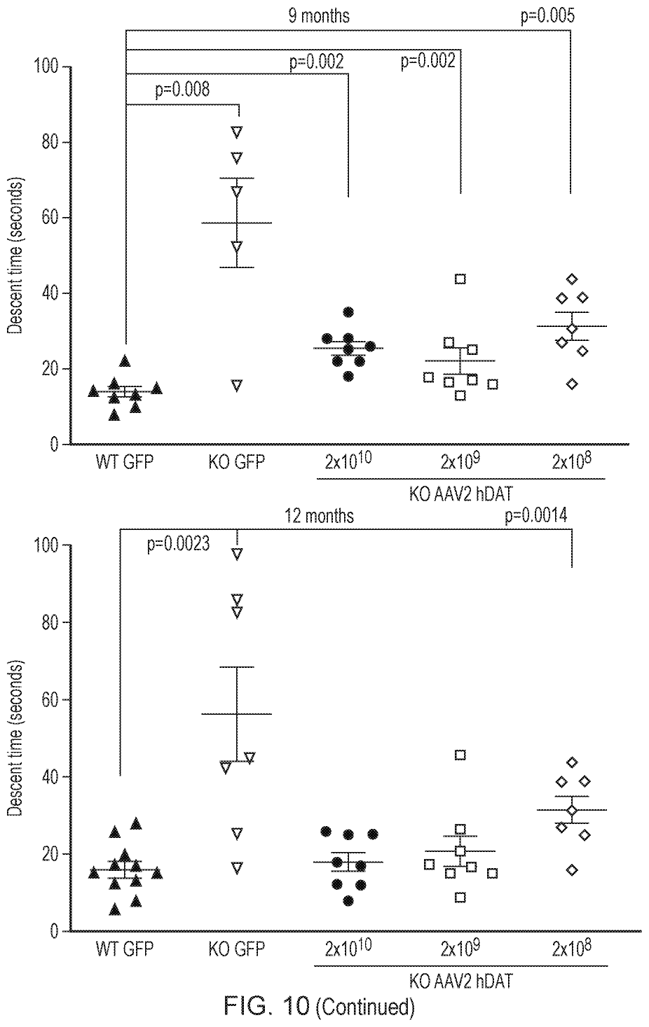

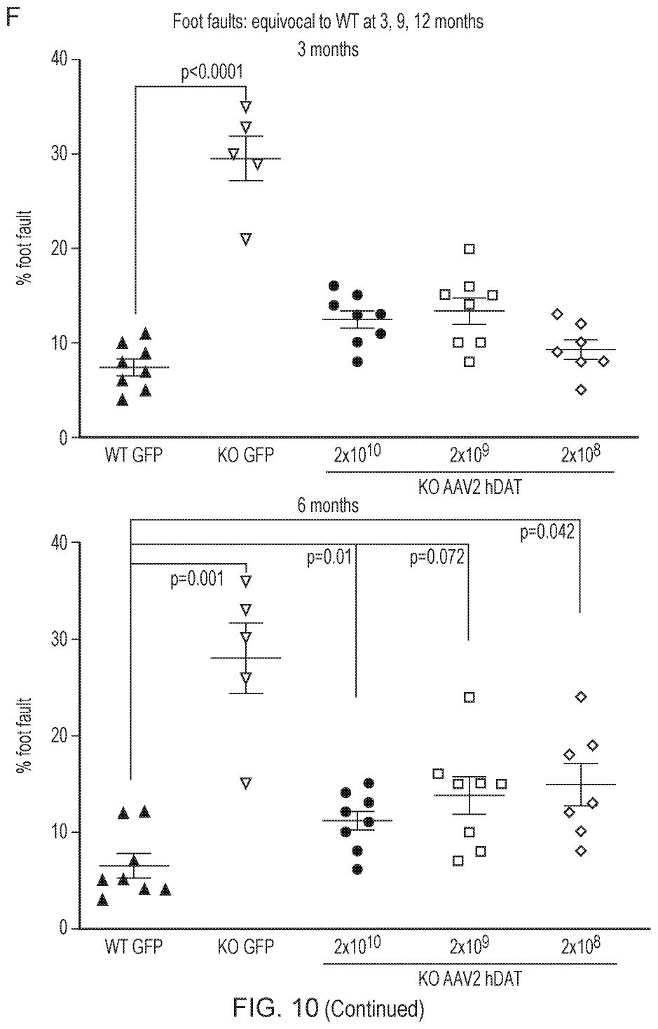

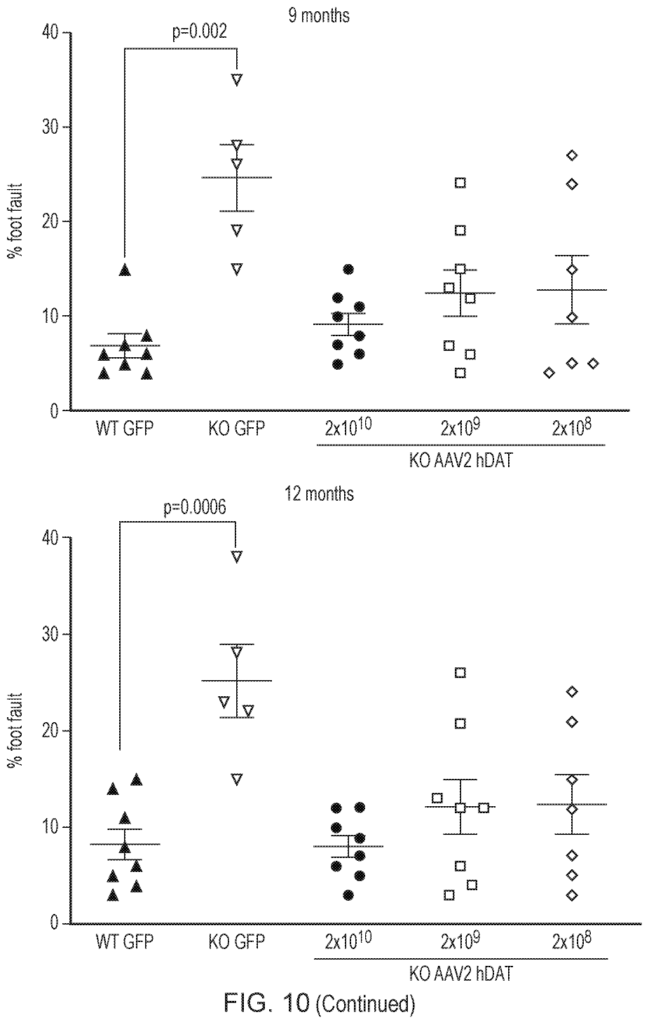

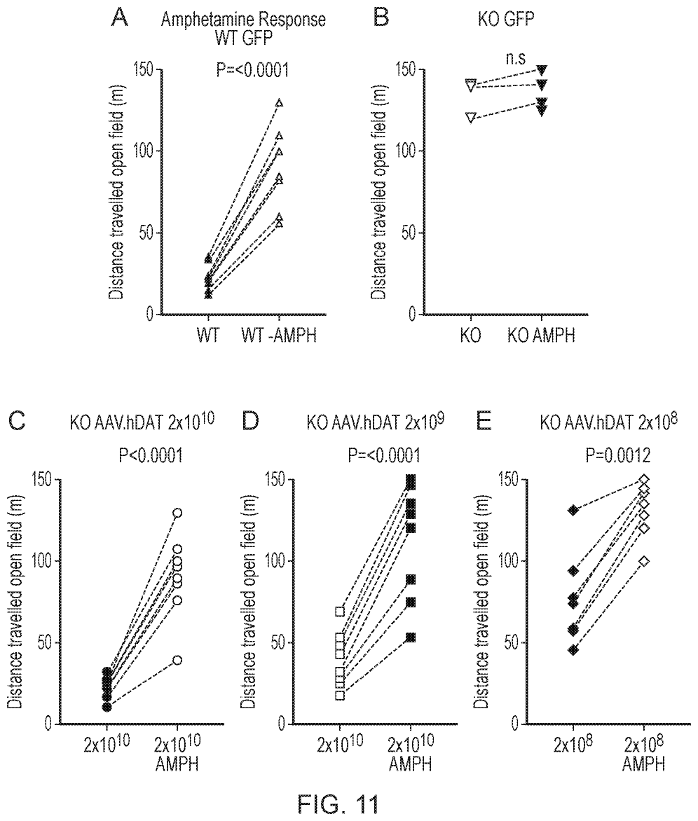

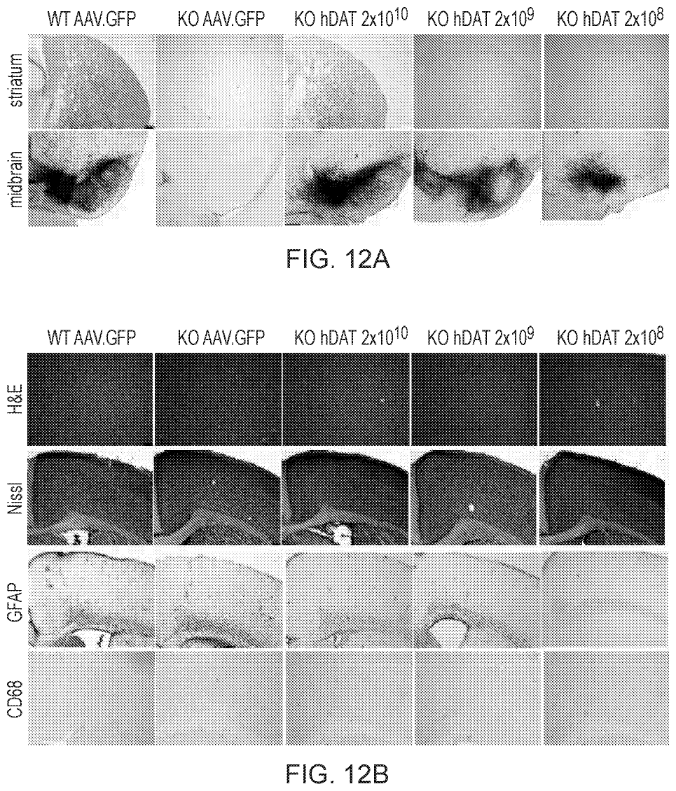

KO mice (KO-eSyn) received single stranded AAV9.eSyn.SL6CA3.WPRE (titre 3.8×10¹³ genomic copies per mL=1.9×10¹⁰ per dose) (FIG. 9A). The KO-eSyn (n=16) were maintained with WT (n=12) and untreated KO (n=12) littermates as controls and followed for 12 months.

All pups were weighed daily for 7 days and then weekly thereafter. The untreated KO exhibited poor weight gain with 7/12 exhibiting a KO-S phenotype dying by P35 and 4 untreated KO mice were alive at 12 months. 4/16 KO-eSyn mice exhibited tremor and weight loss between P14-28 (2 at P14, 1 at P21 and 1 at P28) and the remaining 12/16 KO-eSyn mice had normal survival. This showed survival of KO-eSyn mice (75%) compared to untreated KO (41%), A Kaplan-Meier Curve survival comparison indicated improved survival (Log rank test p=0.02) (FIG. 9B).

Initially the weights of the KO-eSyn and KO were the same but from 2 months the KOeSyn (15.3 g±1.5) gained more weight than untreated KO (14.05 g±0.58). There was no significant difference between KO-eSyn (29.8 g±2.87) and WT mice (31.44 g±1.76) (FIG. 9C).

KO-eSyn mice were assessed at 3 months for effects of gene therapy on motor function with untreated KO and WT mice (n=8 per group). The distance travelled in open field in KO-eSyn was 90.7 cm±10.4 compared to untreated KO 155.3 cm±16.1 with WT distance travelled was 76±9.9 cm. Post hoc Bonferoni analysis showed no difference between WT and KO-eSyn (p=0.12) and significant difference WT versus untreated KO (p=0.005) (FIG. 9D). The distance travelled on exercise wheel was 186.7 cm±33.9 in KO-320.6 cm±51.8 eSyn, untreated KO and 132.5 cm±31.5 in WT (FIG. 9E). There was no significant difference between KO-eSyn and WT (p=0.06) and significant difference between WT and untreated KO only (p=0.0031). The KO-eSyn mice showed reduced Vertical pole T-times (10.3±3.6) compared to untreated KO (53.9 s±20.1) and were not statistically different to WT mice 6.3 s±2.8. There was no significant difference between KO-eSyn and WT (p=0.06). Post hoc Bonferoni analysis showed significant difference between WT and untreated KO only (p=0.0012) (FIG. 9F). On foot fault assessment the KO-eSyn mice showed reduced mean % foot faults (18±3.49) compared to untreated KO 26%±3.61 but higher than WT. Percentage data were transformed to normal distribution with inverse arcsine transformation and analysed with One way ANOVA and post hoc Bonferoni showing difference between WT and KO (p=0.001) and WT and KO-eSyn mice (p=0.005) (FIG. 9G).

At 3 months, 3 animals per group (KO-eSyn, untreated KO and WT) were collected for immuno-histochemical analysis of hDAT expression in the brain. These were stained with KO-eSyn mice that survived between P14-28. The KO-eSyn for IHC for hDAT on whole brain sections showed expression in the cortex, striatum, hippocampus and midbrain but not in the cerebellum. No expression of hDAT was observed in untreated KO mice. The staining morphology at higher magnification revealed punctate staining morphology suggestive of expression in a glial cells rather than neurons (FIG. 9H) but transduction of midbrain dopaminergic neurons was observed on immuno-histofluorescence (FIG. 9I).

### Discussion

Personalised medicine strategies are increasingly important in drug development, particularly for inherited neurodegenerative disorders, where the mainstay of current treatment is symptom control and palliative care. Through the synergistic use of an in vitro iPSC-derived neuronal system and in vivo murine model, this study has not only provided further insight into the underlying mechanisms governing human disease, but has also facilitated the evaluation of novel therapeutic strategies for this pharmacoresistant condition. Both the iPSC-derived mDA model and DAT knockout mouse recapitulate important DTDS disease features, with loss of DAT activity, abnormally raised dopamine metabolites and neurodegeneration. The knockout murine model also exhibits key motor features akin to those seen in human patients, with early hyperkinesia evolving into late-stage parkinsonism. Previous studies of DTDS missense variants (which account for 76.6% of DTDS patient mutations) have utilised cell-based overexpression model systems, Caenorhabditis elegans and Drosophila melanogaster DAT mutants. The inventors' iPSC-based platform provides a new DTDS model with a number of advantages: it allows the study of patient-relevant DAT mutations in a humanized neuronal model system, including variants that cannot be studied in other models, such as L368Q which confers lethality in the fly model. By combining the iPSC and murine disease-relevant models, the inventors have gained further pathophysiological insight into the consequences of loss of DAT function. Both the mDA cell model and knockout murine dissected midbrain show significant reduction of key enzymes in dopamine catabolism, MAO-A and MAO-B suggesting a compensatory downregulation in the absence of dopamine reuptake. Dysregulation of MAO-A and MOA-B was not evident in patient CSF, though it is likely that CSF measurement does not represent midbrain MAO-A/MAO-B enzyme levels. Despite extensive phenotypic rescue of both iPSC and mouse models, viral vector-mediated restoration of DAT activity did not fully restore midbrain MAO levels. These studies reflect that MAO regulation is not solely influenced by dopamine reuptake. In the knockout mouse model, the inventors also observed loss of the normal bimodal firing pattern in the medium spiny neuron population, suggesting that DAT deficiency in mDA neurons may have more widespread detrimental systemic effects on synaptic connectivity and post-synaptic neuronal networks.

In both the mDA neuronal system and mouse model, neurodegeneration is a feature. Although there is limited evidence in DTDS patients, the progressive nature of clinical disease and serial DATscan imaging also both point to a neurodegenerative process. From the iPSC-derived mDA model, the inventors can postulate that neuronal loss may be mediated by an oxidative stress response secondary to extracellular dopamine toxicity with proinflammatory cytokine-induced apoptosis. This is further corroborated by the inventors' findings of raised TNF levels in the CSF of older DTDS patients with more advanced disease. Overall, it is likely that the mechanisms governing neurodegeneration in DTDS are multifactorial; apoptosis may be driven by factors such as dopamine toxicity and oxidative stress, possibly accelerated by the release of proinflammatory cytokines from activated glia.

The inventor's study also highlights the therapeutic limitations of agents such as pifithrin-μ with its mutation-specific chaperone effects, and in contrast, the great potential of gene therapy for all patients with DTDS, with clear evidence of phenotypic rescue in both the cell and knockout mouse model. In the absence of a humanized knock-in mouse model, the inventors' iPSC-derived neuronal model provides crucial clinically-relevant information regarding potential dominant-negative phenomena. Indeed, antagonistic effects from co-expression of both the endogenous mutant allele and wildtype transgene were not observed in the lentivirus-treated cells.

From the inventors' study, it is clear that the neuropathological consequences of DTDS are likely to occur early in life. It is universally acknowledged that despite the maturation process, iPSC-derived neurons resemble fetal neurons and indeed the severe cellular phenotype evident in the DTDS mDA cell model suggests prenatal disease onset. The knockout mouse corroborates this, where poor growth and an early progressive motor phenotype with neuronal loss is observed.

The inventors initially sought to explore neonatal gene therapy, given its significant therapeutic potential for this early onset neurodegenerative disease. Despite variable gene expression in target mDA neurons, the inventors' neonatal gene therapy approach successfully restored DAT function and dopamine homeostasis, providing significant therapeutic impact in the murine model, with restoration of lifespan and motor phenotype. Gene therapy also prevented neuronal loss in the SN, and had beneficial effects on the post-synaptic neuronal network, preventing neuronal loss and normalising electrophysiological properties of the medium spiny neuron population. Although there was evidence of off-target transduction, ectopic overexpression of DAT appeared to be well-tolerated. However, at a ten-fold higher vector dose, the inventors observed off-target neurotoxicity, with astrogliosis in cortical regions and a substantial reduction in survival. Neurotoxic effects and reduced survival have been similarly observed in DAT over-expression and ectopic expression transgenic models. Overall, this strongly suggests that although a low level of ectopic expression is tolerated, ectopic expression should ideally be avoided for clinical translatability.

The study of Illiano et al. provided proof-of-concept for gene therapy of DAT deficiency. They delivered two AAV vectors into the midbrain of adult DAT mice by stereotactic injection. To achieve high specificity for dopaminergic neurons, the first AAV expressed Cre recombinase under the control of the truncated rat tyrosine hydroxylase promoter and a second AAV contained murine DAT flanked by loxP sites, under the control of constitutive CMV promoter. Cre recombinase expression thus permitted specific therapeutic DAT expression. Despite this proof-of-concept, such an approach would not be clinically translatable, with the use of murine DAT and potential neurotoxicity of Cre recombinase expression.

Given that both neonatal intracerebroventricular gene delivery (with risk of potential neurotoxic off-target effects) and the dual AAV vector delivery system described above (with neurotoxic Cre recombinase) are not suitable for clinical translation, the inventors then sought to develop a clinically applicable gene therapy approach for DTDS patients. Their revised gene therapy strategy, using an AAV2 vector, specifically targeting the DAT-expressing SN of the brain serves as an ideal preclinical basis for clinical translatability. The inventors demonstrated efficacy of the therapeutic expression cassette containing a truncated human promoter and human DAT, in vitro in the patient-derived dopaminergic neuronal cell model, primary knockout neurons and in vivo at different developmental ages of the knockout mouse model. Importantly towards clinical translation, the inventors have also demonstrated clinical feasibility with a 2 log dose-ranging study of AAV2.hDAT showing clear (dose-dependent) therapeutic efficacy and also minimised risk of neurotoxic effects from ectopic hDAT expression.

Material and Methods

iPSC Generation and Maintenance

Generation of iPSCs from patient dermal fibroblasts was approved by the Local Research Ethics Committee (Reference 13/LO/0171). Written informed consent was obtained from all patients. Fibroblasts were cultured from skin biopsies and maintained in DMEM (Gibco), 10% FCS (Gibco), 2 mM L-glutamine (Gibco), 1% MEM non-essential amino acids (Gibco), and 100 u/ml penicillin 100 μg/ml streptomycin (P/S, Gibco). Age-matched healthy control fibroblasts were obtained from the MRC Centre for Neuromuscular Disorders Biobank. Fibroblasts were reprogrammed using the commercially available CytoTune®-iPS Reprogramming kit (Invitrogen), containing four CitoTune® Sendai reprogramming vectors (hOct3/4, hSox2, hKlf4, hc-Myc). Viral transduction was performed on cells at 90% confluency (1-1.5×10⁵/well), in 12 well plates. After 6 days, infected cells were harvested with TrypLE™ (Invitrogen) and 8,000 cells/6 well plate were seeded onto gamma-irradiated mouse embryonic fibroblasts (MEF). One day later, medium was changed to knockout-DMEM (Gibco), 20% serum replacement (Gibco), 2 Mm L-glutamine, 50 μM 2-mercaptoethanol (Gibco), MEM non-essential amino acids, P/S, and 10 ng/ml bFGF (Gibco). From day 13, MEF-conditioned medium was added to the culture. Colonies with iPSCs-morphology developed around 30 days after transduction. Eight to ten independent colonies per patient were collected and expanded by manual passaging. Between passage 15 and 20, 3 colonies per patient were converted to mTeSR1 medium (Stemcell t m technologies) onto Matrigel® (Corning®) coated plates. Derived iPSCs were maintained in mTeSR1 on matrigel and regularly passaged with EDTA 0.02% solution. Two colonies per patient (Patient 1-03 and Patient 1-08; Patient 2-01 and Patient 2-06) and age-matched healthy control (Control-05 and Control-03) were characterized and differentiated into mDA neurons to exclude clonal variability. One clone per patient and age-matched healthy control was used for further studies unless otherwise stated.

Generation of Isogenic Control by CRISPR/Cas9 Gene Editing

For DTDS patient line Patient 2-01 harbouring homozygous SLC6A3 variant c.1184C>T, a CRISPR/Cas9 corrected line (CRISPR) was generated by Applied StemCell, Inc (Milpitas, CA). Briefly, two guide RNA (gRNA) candidates were cloned and tested in HEK293 cells to evaluate Cas9-mediated cleavage efficiency in vitro. Patient 2-01 iPSCs were transfected (Neon transfection system, Invitrogen) with gRNAs and single-stranded oligo donor (ssODN). Single cells were seeded in 96-well plates and cultured for 14 days before expanding and culturing in 24 well plates. Each clone was isolated and genomic DNA extracted for PCR amplification of the mutated sequence. PCR products were subsequently sequenced to confirm bi-allelic correction of the homozygous SLC6A3 mutation.

Direct Sanger Sequencing

DNA from all iPSCs lines was extracted using a commercially available kit (DNeasy Blood & Tissue kit, Qiagen), following manufacturer instructions. Direct Sanger Sequencing of genomic DNA extracted from control, patient-derived, and CRISPR-corrected isogenic iPSCs was undertaken to confirm genotype. Primer pairs for exon-specific PCR amplification were designed using Primer3 software (http://bioinfo.ut.ee/primer3/), and the SLC6A3 DNA template (Ensembl genome browser: http://www.ensembl.org/index.html, NCBI Genome Reference Consortium (GRC)h38.p10;chromosome 5: 1,392,790-1,445,430; NM_001044.4). PCR products were purified with MicroCLEAN (WebScientific). The purified PCR product was subsequently sequenced in both forward and reverse directions using the BigDye Terminator Cycle Sequencing System (Applied Biosystems). Sequencing reactions were carried out on an ABI PRISM 3730 DNA Analyzer (Applied Biosystems). The results were analyzed using Sequencher (https://www.genecodes.com) and Chromas software (http://technelysium.com.au/wp/chromas)

Assessment of Genome Integrity

Genome integrity was assessed by Illumina Human OmniExpress24 array using genomic DNA, as per manufacturer's instructions, and Karyostudio software was used to generate karyograms (Illumina).

Analysis of Pluripotency by In Vitro Spontaneous Differentiation

Embryoid bodies (EBs) were generated by harvesting cells with TrypLE™ and plated onto non-adherent bacterial dishes to a concentration of 1.5×10⁵ per cm 2 in knockout-DMEM medium, 20% serum replacement, 2 mM L-glutamine, 1% MEM non-essential amino acids, 50 μM 2-mercaptoethanol (Gibco), 1 μM ROCK-inhibitor (thiazovivin for the first 2 days, Cambridge Bioscience). In order to direct neuroectodermal and endodermal fate, EBs were plated at day 4 on matrigel-coated dishes and maintained in the same media (described above for EB generation) until day 16. For mesodermal differentiation, EBs were plated onto 0.1% galantine (Sigma-Aldrich) coated dishes in DMEM, 20% FCS, and 2 mM L-glutamine for 16 days, until cells were analyzed by immunofluorescence (see below).

Analysis of Pluripotency by Epi-Pluri-Score

All derived iPSCs lines were additionally analyzed with Epi-Pluri-Score (Cygenia), which compares pluripotent with non-pluripotent cells. The Epi-Pluri-Score is based on the combination of DNA methylation levels at the two CpG sites of ANKRD46 and C14orf115.

Differentiation of iPSC in mDA Neurons

iPSCs were differentiated into dopaminergic neurons using a modified version of the dual SMAD inhibition protocol (A. Kirkeby et al., Cell Reports 1, 703-714 (2012); D. Lehnen et al., Stem Cell Reports 9, 1207-1220 (2017)). Briefly, iPSCs were harvested using TrypLE™ (Invitrogen), and plated onto non-adherent bacterial dishes to a concentration of 1.5×10⁵ per cm 2 to generate EBs in DMEM/F12:Neurobasal (1:1), N2 (1:100), B27 minus vitamin A (1:50) (Invitrogen), 2 mM L-glutamine and ROCK-inhibitor (Thiazovivin) for the first two days. EBs were plated on day 4 onto polyornithine (PO; 15 μg/ml; Sigma), fibronectin (FN; 5 μg/ml Gibco) and laminin (LN; 5 μg/ml; Sigma) coated dishes in DMEM/F12:Neurobasal (1:1), N2 (1:200), B27 minus vitamin A (1:100), 2 mM L-glutamine. From day 0 to day 6, media was supplemented with: 10 μM SB431542 (Tocris Bioscience), 100 nM LDN193189 (Stemgent Inc.), 0.8 μM CHIR99021 (Tocris Bioscience) and 100 ng/ml hSHH-C24-II (R&D Systems). On day 2, 0.5 μM purmorphamine (Cambridge Bioscience) was added. SB431542 was withdrawn on day 6 and all other supplements were continued until day 9 of differentiation. On day 11, cells were either processed for midbrain precursor analysis or harvested with Accumax and re-plated on PO/FN/LN coated dishes in droplets of 1-1.5×10⁴ cells per μl in Neurobasal B27 minus vitamin A (1:50), 2 mM L-glutamine, 0.2 mM ascorbic acid (AA) and 20 ng/ml BDNF (Miltenyi Biotech). On day 14 of differentiation, 0.5 mM dibutyryl c-AMP (Sigma-Aldrich) and 20 ng/ml GDNF (Miltenyi Biotech) were added. On day 30 of differentiation, cells were re-plated (following the same protocol as described for day 11) in the same medium, and the γ-secretase inhibitor DAPT (10 μM, Tocris) was added until day 65 of differentiation, when cells were harvested for further analysis.

Immunocytochemistry

Cells were fixed in 4% paraformaldehyde Immunofluorescence (IF) for assessment of pluripotency, spontaneous in vitro differentiation experiments and day 11 mDA precursors was performed in 0.1% triton X-100, 10% fetal calf serum (FCS), 1× phosphate-buffered saline (PBS) except for the surface antibodies TRA-1-60 and TRA-1-81 where triton X-100 was omitted. Immunostaining of samples at day 65 of differentiation was performed in buffer solution with 0.3% triton X-100, 10% FCS, 1× PBS, except for the anti-DAT antibody where 10% normal goat serum was used instead of FCS. After blocking for 30 min at room temperature, all primary antibodies were incubated overnight at 4° C. Cells were then washed three times with 1×PBS and incubated with the respectively species-specific secondary antibodies labelled with Alexa 488, Alexa 594 or Alexa 647 (all from Invitrogen), for 45 min at room temperature. Nuclei were stained with DAPI for 5 min at room temperature. Cells, which had undergone differentiation for 65 days, were seeded on Lab-Tek slides (Nunc™). After immunofluorescence, coverslips were mounted with ProLong Gold Antifade Mountant (Invitrogen).

Imaging was performed with the Olympus IX71 inverted TC scope for assessment of pluripotency markers in iPSC, spontaneous in vitro differentiation of iPSC and day 11 mDA precursors. A multiphoton confocal microscope (Zeiss LSM880) was used for all other IF studies. A Zeiss Axioplan microscope was used for bright-field microscopy. For quantification, 4 random fields were imaged from each independent experiment, and either 1200 or 1800 randomly selected nuclei, depending on the analysis, were quantified using ImageJ software (National Institutes of Health).

Quantitative Real Time PCR (qRT-PCR) Analysis

RNA was purified from cells using the RNeasy mini kit (Qiagen) following the manufacturer's instructions. Contaminating DNA was removed from total RNA (1 μg) using the DNAseI purification kit (Invitrogen), before performing reverse transcription using Superscript III (Invitrogen) to generate cDNA. Sendai virus clearance PCR was performed using manufacturer-recommended oligomers (Invitrogen).

qRT-PCR analysis was performed using the StepOnePlus™ Real-Time PCR System (Applied Biosystems. The qRT-PCR reaction was prepared using 1×MESA Blue qPCR MasterMix Plus for SYBR® Assay (Eurogentec), 0.1 μl ROX Reference Dye (Invitrogen), 9 μL cDNA (dilution 1:25) and 500 nM of each primer (Table 3). All reactions were performed in technical triplicates using the following conditions: denaturation of 95° C. for 5 minutes, followed by 40 cycles of 15 seconds denaturation at 95° C. and 1 minute annealing/extension at 60° C. Relative quantification of gene expression was determined using the 2^{-ΔΔCt} method with glyceraldehyde-3-phosphate dehydrogenase (GAPDH) as a housekeeping reference gene, and normalized to age-matched control lines. In order to assess ubiquitous expression of the mDA marker PITX3, 10 samples at day 65 of differentiation for lines C-05, P1-03, P2-01 and CR-18 were processed. The distribution of mRNA levels was then examined for normality with D'Agostino Pearson, Shapiro-Wilk and Kolmogorow Smirnov statistical tests.

In Vitro Electrophysiology

Current-clamp recordings were undertaken on mDA at day 65 after differentiation, the internal solution contained (in mM): 126 K-gluconate, 4 NaCl, 1 MgSO₄, 0.02 CaCl₂), 0.1 BAPTA, 15 glucose, 5 HEPES, 3 ATP-Naz, 0.1 GTP-Na, pH 7.3. The extracellular (bath) solution contained (in mM): 2 CaCl₂), 140 NaCl, 1 MgCl₂, 10 HEPES, 4 KCl, 10 glucose, pH 7.3. D-(−)-2-amino-5-phosphonopentanoic acid (D-AP5; 50 μM), 6-cyano-7-nitroquinoxaline-2,3-dione (CNQX; 10 μM) and picrotoxin (PTX; 30 μM) were added to block synaptic transmission. Experiments were performed at room temperature (22-24° C.). Neurons with unstable resting potential (or >−50 mV), bridge-balance >20 MΩ and/or holding current >200 pA were discarded. Bridge balance compensation was applied and the resting membrane potential was held at −70 mV. Spontaneous action potentials (APs) were triggered holding the neurons around −60 mV/−55 mV. Current steps protocol was used to evoke APs injecting 500 ms long depolarizing current steps of increasing amplitude (Δ 10 pA). Neurons with repetitive oscillatory spontaneous APs and repetitive evoked APs were considered to be functional mature dopaminergic neuron (10-20% of patched neurons). Recordings were acquired using a Multiclamp 700A amplifier (Axon Instruments, Molecular Devices) and a Power3 1401 (CED) interface combined with Signal software (CED), filtered at 10 kHz and digitized at 50 kHz.

Tritiated Dopamine Uptake Assay

[³H]Dopamine (³H-DA) uptake measurements were performed on derived mDA neurons at day 65 of differentiation in 12 well dishes as described previously (M. E. Reith et al., Methods in Enzymology 296, 248-259 (1998)). Briefly, cells were washed three times in Dulbecco's phosphate-buffered saline with calcium and magnesium (D-PBS+Ca+Mg) (Invitrogen). ³H-DA (Perkin Elmer) was diluted in D-PBS+Ca+Mg to 10 nM with or without 10 μM mazindol (Sigma). Cells were incubated for ³H-DA solution for 15 minutes. The reactions were stopped by adding ice-cold D-PBS+Ca+Mg. Cells were washed twice more and sodium hydroxide (NaOH) added to lyse cells for 1 hour at room temperature. Cells were scraped and transferred into scintillation vials and 1 ml of scintillation fluid (Perkin Elmer) added. Radioactivity was quantified using a scintillation counter (Beckman Coulter). Results were normalized to protein content measured in a sample of the cell lysate using the bicinchoninic acid (BCA) method.

High Performance Liquid Chromatography (HPLC)

Phenol red free media was collected from day 65 mDA neurons and mixed with perchloric acid to a final concentration of 0.4 M. Samples were incubated for 10 min at 4° C. in the dark, centrifuged at 12000×g for 5 min at 4° C., and supernatant was collected for analysis by HPLC.

Mouse brains were harvested immediately following transcardial perfusion with PBS. The right hemisphere was harvested and snap frozen on dry ice for brain homogenisation. The brains were collected and weighed. The hemisphere was transferred to cold glass tissue homogenizer on wet ice and 8× volume of Homogenisation buffer (2 mL 0.8M perchloric acid, 40 μL EDTA 0.1 mM and 6 mL H₂O) was added. The tissue was homogenised in glass homogeniser in wet ice. The brain homogenates were transferred into a 1.5 mL Eppendorf using a Pasteur pipette. The homogenate was incubated at 4° C. then centrifuged at 13000 rpm for 5 minutes before analysis. Dopamine, DOPAC, HVA, and HIAA were quantified using reverse-phase HPLC (C. de la Fuente et al., Neurochemistry International 109, 94-100 (2017)). Briefly, the column consisting on silica with 18 carbon chains was maintained at 27° C. and the flow rate was kept at 1.5 ml/min. The mobile phase was aqueous with 16% methanol, 20 mM sodium acetate trihydrate (pH 3.45), 12.5 mM citric acid monohydrate, 0.1 mM EDTA sodium and 3.35 mM 1-octanesulfonic acid. The detection electrode (Coulochem 2015) was maintained at 450 mV and the screening electrode at 20 mV were injected the system. Peak areas, from the electrochemical detector, were quantified with EZChrom Elite™ chromatography data system software, version 3.1.7 (JASCO UK Ltd).

Immunoblotting

Proteins were extracted from cells and mouse brain tissue in ice-cold RIPA lysis and extraction buffer (Sigma-Aldrich) supplemented with protease inhibitor (Roche). Protein concentration was measured with Pierce™ BCA Protein Assay kit (Thermo Scientific): 10 μg of protein was denatured with Laemmli buffer (Bio-Rad Laboratories LTD) with dithiothreitol (DTT). Proteins were separated with Mini-PROTEAN TGX Stain Free Gels (Bio-Rad Laboratories LTD) and transferred to a Trans-Blot Turbo Transfer membrane (Bio-Rad Laboratories LTD). After blocking in 5% milk, 1×PBS, 0.1% Tween for 1 h at room temperature, membranes were incubated with primary antibodies (Table 2) at 4° C. overnight. Membranes were then incubated with the secondary anti-rabbit horseradish peroxidase-conjugated antibody at a dilution of 1:3000 (Cell Signalling). Immunoreactive proteins were visualized with Chemidoc MP (Bio-Rad Laboratories). In order to evaluate the total amount of endogenous protein and control for equal loading, membranes were reprobed for GAPDH, after clearance with Restore Western Blot Stripping Buffer (Thermo Scientific). CSF sample protein concentrations were measured with Pierce™ BCA Protein Assay kit (Thermo Scientific) and 10 μg denatured with Laemmli buffer with dithiothreitol (DTT). Human Transferrin was probed in CSF for equal loading. The intensity of immunoreactive bands was analyzed using ImageJ software (National Institutes of Health). The density of the bands was normalized to GAPDH. Results are reported as means±SEM of independent experiments, the number of which is stated for each experiment in the respective figure legend.

Treatment of Neuronal Cultures with Pifithrin-μ

Derived mDA neurons at day 65 of differentiation were treated with 1 μM pifithrin-μ (Sigma-Aldrich) for 24 hours. Medium was subsequently removed and the uptake of ³H-DA was assessed as described above.

Lentiviral Vector Generation

The human DAT coding sequence (NM_001044.4) and human Synapsin 1 promoter (S. Kugler et al., Gene Therapy 10, 337-347 (2003)) were cloned into a pCCL lentiviral expression vector (T. Dull et al., Journal of Virology 72, 8463-8471 (1998)) using standard cloning methods. To facilitate identification of transduced cells, an internal ribosomal entry site (IRES2) and enhanced green fluorescent protein (EGFP) coding sequence were then inserted downstream of the DAT sequence. Control plasmid was generated using the CCL-hSyn.IRES2.GFP.WPRE as a template with the primers 3F and 3R. VSV-G pseudotyped lentiviral vectors (LV) were produced using a 2^{nd} generation packaging system (T. Dull et al., Journal of Virology 72, 8463-8471 (1998)). For virus titration, 1×10⁵ HeLa cells were plated into each well of a 6 well plate and transduced with a range of volumes of the concentrated lentivirus. Seventy-two hours after transduction, HeLa cell genomic DNA was extracted and the proviral titre was calculated by qPCR, as described previously (S. Charrier et al., Gene Therapy 5, 479-87 (2011)). Titres ranged 7×10⁸-2×10⁹ vg/mL. As described, mDA neural cells were differentiated for 23-29 days, before transduction with LVVs at the designated multiplicity of infection (MOI). LV-containing media was replaced with fresh culture medium 24 hours after transduction.

AAV Vector Generation

hSyn.GFP plasmid containing single-stranded AAV2 inverted terminal repeats was obtained from Addgene (105539) and used to generate the control AAV vectors. The human hDAT cDNA was cloned into this AAV expression vector using standard cloning techniques. Recombinant single stranded AAV2/9 (referred to as AAV9, throughout) and AAV2 serotype vectors encoding hDAT or GFP were generated by the standard triple plasmid transfection method as described previously (C. J. Binny et al., Methods Molecular Biology 891:109-131(2012)). Cell lysates of transfected 293T cells and vector purified through affinity chromatography on an ÄKTAprime plus (GE Healthcare Ltd, UK) with Primeview 5.0 software with a POROS™ CaptureSelect-™ AAVX resin (Thermo Fisher Scientific, Germany). All vector preparations were titred by RT-qPCR using the Applied Biosystems StepOne Plus Real-Time PCR system. 5 μl of AAV vector was digested in 45 μl DNAse I buffer and 10 units DNAase I (NEB) and incubated at 37° for 1 hour. PCR reactions were performed in 20 μl of final volume using the Luna Taqman qPCR mix (NEB). Primers and probe used targeted transgenes GFP or hDAT serial dilutions of linearized plasmid to generate a standard curve. All vectors were produced to titres 1×10¹³-1×10¹⁴ vg/mL.

Animal Welfare

All animal experiments were performed in compliance with UK Home Office and the Animal (Scientific Procedures) Act of 1986, and within the guidelines of University College London ethical review committee. Outbred CD1 dams (Charles River, UK) time-mated to generate P0-P1 litters for marker gene studies. Pups were weaned at P21 and euthanised for tissue analysis at P35.

The DAT knockout mouse model used in this study has been described previously (K. Hyland et al., Pediatr Res. 1;10-14 (1993); A. Kasture et al., The Journal of Biological Chemistry 291, 20876-20890 (2016)). Heterozygous mice were time mated to generate mixed genotype litters. Pups were genotyped at P0 using primers. Intracerebroventricular gene therapy was delivered to knockout pups by P1.

Neonatal Intracerebroventricular Injection

The intracerebroventricular injections were directed to the lateral ventricle of P0-1 mice as described previously (J. Y. Kim et al., Journal of visualized experiments: JoVE, 51863 (2014)). A 33-gauge needle (Hamilton) was inserted perpendicularly at the injection site to a depth of 3 mm and 5111 of vector was administered over 5 seconds into the lateral ventricle. The pup was returned to dam promptly.

Adult Stereotactic Injection

Animals underwent stereotactic surgery at 28-30 days post-natal days. Mice were anaesthetised in induction chamber with Isoflurane/O2 mixture at a ratio of 3:2. The head was shaved and mice were placed in a stereotactic frame (Panlab, Harvard Apparatus) on homeothermic heating mat system (Panlab, Harvard Apparatus). Anaesthesia was maintained by continuous nose cone isoflurane/O2 mixture at 2.5:2.5. Mid-line scalp incision was made and burr holes drill with hand microdrill (Panlab, Harvard Apparatus, USA). Injections bilaterally targeted the SN antero-posterior (AP) −3.2 mm, medio-lateral (ML) ±1.2 mm relative to the Bregma and dorso-ventral (DV) 4.3 mm relative to the dural surface (G. Paxinos, K. F. Paxinos Sao Paulo, Academic Press 360 p. https://www.elsevier.com/books/paxinos-and-franklins-the-mouse-brain-in-stereotaxic-coordinates/paxinos/978-0-12-391057-8 (2012)). AAV2 vectors were delivered through 33 gauge Hamilton needle and 5 μl syringe infused at 100 nL per minute and needle withdrawn gradually over 30 minutes. Dosages were injected in 2 μl volume bilaterally (dosage ranging from 2×10⁸ to 2×10¹⁰ vg/mouse). Wound was closed with 4.0 vicryl suture (Ethicon). All animals were single housed and monitored daily for 1 week for general health status. All animals fully recovered from surgery and were all included in the study.

Behavioural Studies

Mice were weighed regularly and assessed for changes in motor phenotype. Spontaneous open field locomotor activity and thigmotaxis were recorded (300 mm width×300 mm length×200 mm height) in an illuminated quiet room for 15 minutes. The distance travelled for 15 minutes was recorded and quantified using motion tracking software (Smart 3.0, Panlab, Harvard Apparatus).

Vertical pole test places mouse upwards facing on the top of a vertical wooden rough surfaced pole (diameter 1 cm, height 50 cm). Each mouse was habituated to the pole on the day prior to testing, then allowed to descend five times on a single session. The total time until the mouse reached the floor with its four paws was recorded. If the mouse was unable to descend or fell or slipped down, the default value of 120 seconds was taken into account. The foot fault test was performed to evaluate the motor accuracy abilities of the mice to place the forepaws on a wire while moving along a metal grid. The mice are placed on raised a metal grid with 10 mm×10 mm square grids (200 mm width×300 mm length) and allowed to spontaneously explore the grid for 5 minutes. The animals were videotaped and the frequencies of slips for the forelimbs and hindlimbs was recorded with total number of steps during locomotion were recorded. A positive foot fault was considered when the paw slip caused the animal to fall between rungs. Video assessors were blinded to genotype and treatment group.

To evaluate the effects of amphetamine or saline on locomotor behaviour distance travelled in open field, mice were habituated for 1 hour before testing. The distance travelled was measured for 15 minutes in open field. Amphetamine was dissolved in saline and administered at 0.1 ml/10 g body weight by intraperitoneal injection and after 30 minutes post drug administration the open field distance travelled was remeasured to assess for amphetamine response.

Histological and Immunohistochemical Analyses of Mouse Tissues

Mice were culled by terminal transcardial perfusion using PBS. Collected tissues (brain and visceral organs) were halved to allow for different processing techniques. Brains used for immunohistochemistry were post-fixed in 4% PFA for 48 hours and transferred into 30% sucrose solution for cryoprotection at 4° C. until sectioning. Brains were mounted on a freezing microtome (Thermo Fisher HM430) at 40 μm thickness in either coronal or sagittal planes. Free-floating immunohistochemistry-based analyses was performed as previously described (A. A. Rahim et al., FASEB journal: official publication of the Federation of American Societies for Experimental Biology 25, 3505-3518 (2011)) with brain sections selected at 240 μm intervals for whole-brain immunohistochemistry. Briefly, free-floating sections were blocked in 15% normal goat serum (Vector Laboratories)-tris buffered saline with 0.1% triton-X (TBS-T) (Sigma) for 1 hour at room temperature and incubated in primary antibodies (Table 2) in 10% normal goat serum-TBS-T overnight at 4° C.: The following day sections are incubated with the respectively species-specific secondary antibodies (Vector Laboratories Inc.) for 1 hour at room temperature, washed in TBS followed by incubation with Vectastain avidin-biotin solution (Vector Laboratories). The reaction visualised with 3,3′-Diaminobenzidine (DAB) (Sigma). DAB reaction was stopped using ice cold 1×TBS and sections washed before mounting on double coated gelatinized glass slides. The mounted sections were air dried and dehydrated in 100% ethanol for 10 minutes and Histoclear (National Diagnostics) for 30 minutes prior to being covered with DPX mountant (VWR International) for coverslipping.

Conventional methods were used for Harris hematoxylin and eosin staining (Sigma-Aldrich). Brain Sections were mounted on chrome-gelatine-coated slides and air-dried overnight. The sections were stained with filtered 0.1% Mayer's haematoxylin (Sigma-Aldrich) for 10 min. The slides were rinsed in distilled water for 5 min and consequently dipped in 0.5% eosin solution. The sections were washed in distilled water and subsequently dehydrated in rising concentrations of ethanol (50%, 70%, 95%, 100%). The slides were coverslipped with DPX mountant (VWR International).

For Nissl staining representative brain sections were mounted onto double coated gelatinized slides and dried overnight. The sections were dehydrated in 70% ethanol overnight on the second day. Slides were immersed in the 1% Cresyl violet solution (Millipore) for 3 minutes. Excess solution was removed by washing twice in running water. The slides were dehydrated by consecutive immersion (2 minutes each) in increasing concentrations of ethanol (70%, 90%, 96%, 96% with glacial acetic acid (Sigma) 100% EtOH, Isopropanol, and three washes in Xylene. Slides were then coverslipped as described previously.

For immunofluorescence brain sections were blocked in 15% goat serum for 30 minutes and then incubated with primary antibodies (Table 2) diluted in 10% normal goat serum TBS-T 0.3% overnight at 4° C. The sections were washed in 1×TBS and incubated for 2 hours with the respectively species-specific secondary antibodies labelled with Alexa 488 and Alexa 594 (all from Invitrogen) diluted in 10% normal goat serum at room temperature. Nuclei were stained with DAPI (Sigma Aldrich) for 2 minutes. The brain sections were mounted onto double coated slides and coverslipped using Fluoromount G (Thermofisher Scientific).

Light microscopy and fluorescence imaging were carried out using a Leica DM 4000 linked to Leica DFC420 camera system. Confocal images were captured using a Leica TCS SP5 AOBS confocal microscope. Images were analysed with Image J software (National Institutes of Health).

Quantification of neurons was conducted with assessor blinded to genotype and treatment group. For each animal, eighteen non-overlapping ×40 magnification images were taken through four consecutive sections for each region of interest striatum and midbrain. During image capture, the same camera and microscope settings were maintained. The average values of cell counting are represented.

Acute Slice Electrophysiology

Untreated knockout, wildtype and treated knockout mice were rapidly perfused with ice cold oxygenated slicing solution (in mM): 75 sucrose, 87 NaCl, 2.5 KCl, 25 NaHCO₃, 25 glucose, 7 MgCl₂, 0.5 CaCl₂. Brains were quickly dissected into ice-cold oxygenated slicing solution and were cut into 300 lam coronal slices using a VT1200S Vibrotome (Leica Biosystems). Slices were stored submerged in oxygenated recording standard aCSF (in mM): 125 NaCl, 2.5 KCl, 25 NaHCO₃, 1.25 NaH₂PO₄·H₂O, 1 MgCl₂, 2 CaCl₂), 25 glucose at room temperature for at least one hour prior to recording. All the current clamp recordings were performed in a standard external solution containing (see slice preparation section above) in presence of D-(−)-2-amino-5-phosphonopentanoic acid (D-AP5; 50 μM), 6-cyano-7-nitroquinoxaline-2,3-dione (CNQX; 10 μM) and bicuculline methiodide (30 μM) for blocking of NMDA, non-NMDA, and GABAA receptors, respectively. The internal solution contained (in mM): 126 K gluconate, 4 NaCl, 1MgSO₄, 0.02 CaCl₂), 0.1 BAPTA, 15 glucose, 5 HEPES, 3 ATP, 0.1 GTP (pH 7.2 with knockoutH). Resting membrane potential was hold at −70 mV for all the recordings. Neurons with leak current >100 pA and Ra >20MΩ were not considered for the analysis. All recordings and analysis were carried blinded to mouse genotypes. Recordings were acquired using a Multiclamp 700A amplifier (Axon Instruments, Molecular Devices, Sunnyvale, CA, USA) and Signal software in conjunction with CED Power 1401-3 (CED, Cambridge Electronic Design Limited), filtered at 10 kHz and digitized at 50 kHz. The sampling frequency was set to 20 KHz. A 500 ms step currents were injected from −20 pA to 300 pA with 10 pA increases. AP were calculated only if they crossed 0 mV and they had a rinsing slope (dV/dt)>20 mV/ms.

Primary Neuron AAV Transduction

Knockout or wildtype breeding pairs were time-mated to generate knockout or wildtype litters. P0 pups were transcardially perfused and brains extracted on wet ice. The neonatal neurons were isolated using the Neural Dissociation Kit and MACS system Neuron isolation kit (Miltenyi Biotech) as per manufacturer's instructions. Neurons were seeded into poly-D-lysine coated coverslips in 24-well plates at density of 1×10⁵ in 50 μl Neural basal medium (Invitrogen), 2% heat-inactivated fetal bovine serum (Sigma-Aldrich), 2% B27 supplement (Invitrogen), 200 mM L-glutamine (Sigma-Aldrich) and 25 mM L-glutamate (Sigma-Aldrich). Cells were rested for 30 minutes at 37° C. and 450 μl of medium was added to each well. Cells were maintained in 5% CO2 incubator at 37° C. replacing 50% medium every 24 hours. Cultures were transduced on day 2 using AAV2.GFP or AAV2.hDAT at MOI 1000-10000 MOI in 5 μl media with 50% media replacement after 24 hours. On day 5, media was exchanged for phenol red free media collected on day 7. The cells were collected on day 7 for HPLC analysis, hDAT immunoblotting or immunofluorescence analysis as described above.

Vector Genome Transcript and qRT-PCR mRNA Transcript Expression Analysis

Genomic DNA was recovered using the DNeasy Blood and Tissue kit (Qiagen) and quantified on Omega Fluostar. For the quantification of GFP or hDAT cDNAs transcripts, standardisation was achieved by comparison against standard curves generated by amplification from plasmid constructs specific for GFP, hDAT and mGAPDH transcripts. This enabled estimation of absolute numbers of transcripts and reference GAPDH gene transcript, using a standard curve in Quantstudio™ Real-Time PCR System (Applied Biosystems).

RNA was extracted from midbrain homogenate extracted with RNeasy mini kit (Qiagen) following the manufacturer's instructions and quantified on Omega Fluostar. Contaminating DNA was removed from total RNA (1 μg) using the DNAse I purification kit (NEB), before performing reverse transcription with High-Capacity cDNA Reverse Transcription Kit (Applied Bioscience). Then 10 ng of DNA or synthesized cDNA was used to perform the multiplex hDAT and mGAPDH RT-qPCR with Luna Taqman mastermix (NEB) in Quantstudio™ Real-Time PCR System (Applied Biosystems). GAPDH was used as endogenous controls and relative fold change calculated.

Statistical Analysis

Statistical analysis tailored to each experiment was performed using GraphPad Prism version 8. For the statistical analysis of iPSCs derived data, when dual comparisons were required two-tailed Student's t-test was applied, while for multiple comparisons one-way analysis of variance (ANOVA) was performed. In vivo experimental design and sample sizes were designed using NC3Rs guidance and power calculation. For most analyses of animal experiments, one-way or two-way ANOVA was performed with either Bonferroni or Tukey's multiple comparison. % foot faults were converted by log transformation before ANOVA. For neuronal firing Kruskal-Wallis test for distribution was applied.

Sequences
SEQ ID NO: 1—human synapsin 1 promoter, truncated version
SEQ ID NO: 2—human synapsin 1 promoter with 5′ extension
SEQ ID NO: 3—human synapsin 1 promoter with 3′ extension
SEQ ID NO: 4—eSYN promoter
SEQ ID NO: 5—human beta-actin promoter
SEQ ID NO: 6—chicken beta-actin promoter
SEQ ID NO: 7—CAG promoter
SEQ ID NO: 8—CMV promoter
SEQ ID NO: 9—CMV promoter (second version)
SEQ ID NO: 10—human CAMKII promoter
SEQ ID NO: 11—human EF1-α promoter
SEQ ID NO: 12—human SLC6A3 gene
SEQ ID NO: 13—amino acid sequence of human DAT protein
SEQ ID NO: 14—CMV immediate early enhancer
SEQ ID NO: 15—TPL-eMLP adenovirus derived enhancer element
SEQ ID NO: 16—Human beta-globin intron
SEQ ID NO: 17—Human beta-actin exon/intron
SEQ ID NO: 18—Human EF1-α intron/exon
SEQ ID NO: 19—Human EF1-α, intron A
SEQ ID NO: 20—Chicken beta-actin exon/intron+rabbit globin intron
SEQ ID NO: 21—CMV IE exon
SEQ ID NO: 22-5′ UTR-Syn1 Hs
SEQ ID NO: 23-5′ UTR human CamKIIa
SEQ ID NO: 24-5′ ITR
SEQ ID NO: 25-5′ ITR
SEQ ID NO: 26-3′ ITR
SEQ ID NO: 27-3′ ITR
SEQ ID NO: 28—AAV2 hSyn1 SLC6A3 vector construct

1. A vector for treating Dopamine Transporter Deficiency Syndrome, the vector comprising a promoter operably linked to a human SLC6A3 gene, wherein the promoter is selected from a human synapsin 1 promoter, a CAG promoter, a CMV promoter, a CAMKII promoter, a beta-actin promoter, and a human EF1-alpha promoter.

2. A vector according to claim 1, wherein the human SLC6A3 gene has the nucleotide sequence of SEQ ID NO: 12 or has at least 70% sequence identity thereto, and encodes a functional DAT protein.

3. A vector according to claim 1 or 2, wherein the human SLC6A3 gene encodes a functional DAT protein having the amino acid sequence of SEQ ID NO: 13 or has at least 80% sequence identity thereto.

4. A vector according to any preceding claim, wherein the promoter is a neuron-specific promoter.

5. A vector according to any preceding claim, wherein the promoter is a human synapsin 1 promoter.

6. A vector according to any preceding claim, wherein the human synapsin 1 promoter is selected from a hSYN1 promoter, an hSYN1 with 5′ extension promoter, an hSYN1 with 3′ extension promoter, an eSYN promoter and a truncated hSYN1 promoter.

7. A vector according to any preceding claim, wherein the vector is an AAV vector.

8. A vector according to claim 7, wherein the vector is an AAV2 vector.

9. A vector according to any preceding claim, wherein the vector is an AAV2 vector comprising a human synapsin 1 promoter operably linked to the human SLC6A3 gene.

10. A vector according to any preceding claim, wherein the vector does not comprise a lox site.

11. A pharmaceutical composition comprising the vector of any of claims 1 to 10 and one or more pharmaceutically acceptable excipients.

12. A method of treating Dopamine Transporter Deficiency Syndrome comprising administering a therapeutically effective amount of a vector according to any one of claims 1 to 10 to a patient with Dopamine Transporter Deficiency Syndrome.

13. The method of claim 12, wherein the vector is administered to dopaminergic neurons by intracranial administration.

14. The method of claim 12 or 13, wherein the vector is administered to dopaminergic neurons of the substantia nigra by intraparenchymal administration.

15. The method of any one of claims 12 to 14, wherein the vector is administered at a single point in time with no repeated administrations.

16. The vector according to any one of claims 1 to 10 for use in therapy.

17. The vector according to any one of claims 1 to 10 for use in the treatment of Dopamine Transporter Deficiency Syndrome.

18. The use of the vector according to any one of claims 1 to 10 in the manufacture of a medicament for treating Dopamine Transporter Deficiency Syndrome.

19. The use of claim 17 or 18, wherein the vector is for administration to dopaminergic neurons by intracranial administration.

20. The use of claim 19, wherein the vector is for administration to dopaminergic neurons of the substantia nigra by intraparenchymal administration.

21. The use of any one of claims 17 to 20, wherein the vector is for administration at a single point in time with no repeated administrations.

22. A host cell comprising the vector of any of claims 1 to 10.

23. A transgenic animal comprising cells comprising the vector of any of claims 1 to 10.
